(19) United States
(12) Patent Application Publication
(10) Pub. No.: US 2012/0053180 A1
(43) Pub. Date: Mar. 1, 2012

(54) CYCLOHEXANE ANALOGUES AS GPR119 AGONISTS

(75) Inventors: Sang Uk Kang, Seongnam (KR); Min Jeong Kim, Seongnam (KR); Byung Nam Kang, Seongnam (KR); Wei Tan, Seongnam (KR); Albert Charles Gyorkos, Westminster, CO (US); Suk Young Cho, Seongnam (KR)

(73) Assignee: CHEMIZON, A DIVISION OF OPTOMAGIC CO., LTD., Seongnam (KR)

(21) Appl. No.: 13/050,801

(22) Filed: Mar. 17, 2011

Related U.S. Application Data
(60) Provisional application No. 61/377,557, filed on Aug. 27, 2010.

(51) Int. Cl. A61K 31/501; A61K 31/496; C07D 239/34; A61K 31/505; C07D 241/04; A61K 31/495; C07D 403/04; C07D 211/96; A61K 31/445; C07D 213/68; A61K 31/4412; C07C 69/22; A61K 31/222; C07C 317/22; A61K 31/085; C07C 43/235; A61P 3/00; A61P 3/10; A61P 9/00; C07D 403/12

(52) U.S. Cl. 514/252.02; 544/295; 514/252.14; 544/316; 514/274; 544/398; 514/252.12; 544/238; 546/242; 514/315; 546/290; 514/345; 560/255; 514/546; 568/33; 514/719; 568/644

(57) ABSTRACT

This invention relates to a series of substituted cyclohexane containing analogues which are agonists of GPR119 intended to treat metabolic diseases mediated by GPR119 including Type I & II diabetes mellitus. Diabetes mellitus is an ever-increasing threat to human health causing various complications (blindness, kidney failure, neuropathy, heart attack, stroke, etc.). Recently it was found that activation of GPR119 which is highly expressed in pancreatic beta cells causes glucose dependent insulin secretion and GLP-1 release. Many pharmaceuticals are currently developing GPR119 agonists and herein we disclose alternative GPR119 agonists. Our invention describes GPR119 agonists having structural Formula (I), pharmaceutically acceptable salt or solvate of Formula (I), isomer or prodrug of Formula (I), and combination therapy of Formula (I) with other anti-diabetic drugs like DPP-IV inhibitors and/or insulin sensitizers.

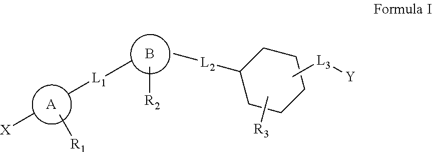

### CROSS-REFERENCE TO RELATED APPLICATION

This application is a non-provisional application claiming the benefit of the filing date of the provisional application of Application No. 61/377,557 filed on Aug. 27, 2010, the entire contents of which is incorporated by reference.

### FIELD OF THE INVENTION

This invention relates to a series of substituted cyclohexane containing analogues which are agonists of GPR119 and are useful in the treatment of Type I and Type II diabetes mellitus including the metabolic disease mediated by GPR119. This invention also relates to a pharmaceutical composition comprising the compound of the invention, use of the compound in the preparation of a medicament including combination therapy with DPP-IV inhibitors and/or insulin sensitizers.

### BACKGROUND OF THE INVENTION

Diabetes mellitus is a condition in which a person has high blood sugar afflicting an estimated 285 million people in 2010 worldwide and this figure is projected to exceed 400 million by 2030. (IDF Diabetes Atlas, 2010). Diabetes mellitus can be divided into two clinical syndromes. Type I diabetes, or insulin-dependent diabetes results from the body's failure to produce insulin due to the extensive loss of beta cells in the pancreatic islets. As these cells are progressively destroyed, the amount of secreted insulin decreases, eventually leading to hyperglycemia. Typically insulin therapy is needed for Type I diabetes.

Type II diabetes mellitus (T2DM), or non-insulin dependent diabetes which is more prevalent than Type I, results from a multiplicity of pathologies, including defective insulin secretion, inappropriate hepatic glucose production and insulin resistance. Genetic and lifestyle factors are central to initiation of the disease, in which insulin resistance is a critical factor. Insulin resistance is a condition wherein muscle, fat or liver cells fail to use insulin properly and this is thought to be due to reduced numbers of insulin receptors on these cells, or a dysfunctioning of signaling pathways within the cells, or both.

Although many attempts have been made to treat diabetes mellitus, currently there is no cure for diabetes mellitus. A first-level of therapy is through the use of diet and/or exercise, either alone or in combination with therapeutic agents. Overall, 49% of patients require oral medication and about 40% of individuals require insulin injections or a combination of insulin and oral medication (Expert Opin. Ther. Patents, 2009, 19(10), 1339-1359).

We can categorize all anti-diabetic drugs into 3 types. First, insulin and its analogues (Lantus by Sanofi-Aventis, NovoRapid by Novo Nordisk) which had 46.1% ($ 10.5 billion) global market share in 2009 but are not orally available (source; Korea Research Institues of Chemical Technology). Second, incretin analogues (Byetta by Amylin/Eli Lilly, Victoza by Novo Nordisk) which have the benefits of stimulating insulin release in a glucose-dependent manner and especially Byetta (Amylin/Eli Lilly) marked about $ 0.7 billion global sales in 2008 (source; Korea Research Institutes of Chemical Technology) but are not orally available either. Third, there are multiple orally available anti-diabetic drugs. For example, Sulphonylureas (Tolazamide, Gliclazide, Glimepiride) act by depolarizing the beta cell; however, they exhibit multiple side effects including hypoglycermia and weight gain. Metformin series which activate AMPK (AMP-activated protein kinase) have side effects of lactic acidosis and gastrointestinal upset. Thiazolidinedione series (Rosiglitazone, Pioglitazone) acting on PPARs (peroxisome proliferator activated receptors) have side effects of liver toxicity, weight gain and risk of heart failure. DPP-IV inhibitors (Januvia by Merck, Onglyza by BMS) inhibit DPP-IV (dipeptidyl peptidase 4) which is responsible for the degradation of incretins. Although there is a possibility of causing cancer in theory, DPP-IV inhibitors are gaining more market share as oral anti-diabetic drugs. Based on the fact that diabetes mellitus is an ever-increasing threat to human health due to aging population, sedentary life style, urbanization and increasing prevalence of obesity, there is a strong need for next generation oral anti-diabetic drugs which have minimal side effects.

Although numerous receptors exist in humans, GPCRs (G protein-coupled receptors) comprise by far the most abundant protein family and are involved in many diseases. According to their abundancy, endogenous ligands of some of them were not identified and are termed “orphan receptors”

GPR119 was one of the such orphan receptors, first identified through bioinformatics, cloned In 2003, and its endogenous ligand was recently found to be oleoylethanolamide (OEA) (British Journal of Pharmacology 2008, 155, 1056-1065, Cell metabolism 2006, 3, 167-175). This receptor is highly expressed in pancreatic islets and in some regions of the gut showing some hints that this receptor might be associated with digestion or modulation of insulin secretion. Indeed, this receptor was first demonstrated convincingly in studies utilizing both in vitro and in vivo assays (Endocrinology 2007, 148, 2601-2609, Journal of Medicinal Chemistry 2008, 51, 5172-5175). After the discovery of GPR119, many pharmaceutical companies have targeted this receptor since this has significant potential as a new target in type 2 diabetes mellitus treatment. GPR119 agonists stimulate incretins release (glucagon-like peptide-1, GLP-1) as well as glucose dependent insulin secretion (Endocrinology 2008, 149(5), 2038-2047). GLP-1 (glucagon-like peptide-1) is produced and secreted from intestinal L cells in response to meals by both direct action of nutrients in the gut lumen and neural stimulation by food ingestion. GLP-1 improves glycemic control by enhancing glucose-dependent insulin release maintaining beta cell mass and more importantly, this promotes satiety and reduced weight gain (Annul Reports in Medicinal Chemistry, vol 44, chapter 7). Because GLP-1 has an extremely short half life in vivo due to DPP-IV (endopeptidase dipeptidase 4), inhibition of this peptidase results in significant therapeutic benefit. In this context, combination therapy of GPR119 agonists with DPP-IV inhibitors would be a good choice as an anti-diabetic treatment regimen. Moreover, based on the fact that diabetes mellitus is related with obesity, GPR119 is an attractive target for obesity treatment as well as diabetes mellitus. In this regard, GPR119 agonists are probably able to represent the next generation oral anti-diabetics and anti-obesity drugs.

### SUMMARY OF THE INVENTION

This invention provides a compound of formula I, or a pharmaceutically acceptable salt, isomer or prodrug thereof:

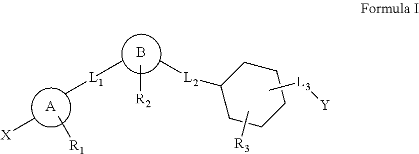

Wherein,

A is 5˜6 membered aryl, heteroaryl, cyclic alkyl and cyclic heteroalkyl;

B is (C)ᵢ where i=0 or 1 independently, and C is independently chosen from 3˜6 membered aryl, heteroaryl, cyclic alkyl, and cyclic heteroalkyl;

L₁, L₂, L₃ are linkers having the formula of (Z)ₖ where k=0 or 1 independently and Z is independently chosen from —(CRₐR_{b})ₘ—, —(CRₐR_{b})ₘO(CRₐR_{b})ₙ—, —(CRₐR_{b})ₘCO(CRₐR_{b})ₙ—, —(CRₐR_{b})ₘS(CRₐR_{b})ₙ—, —(CRₐR_{b})ₘSO(CRₐR_{b})ₙ—, —(CRₐR_{b})ₘSO₂(CRₐR_{b})—, —(CRₐR_{b})ₘNRₐ(CRₐR_{b})ₙ—, —(CRₐR_{b})ₘCONRₐ(CRₐR_{b})ₙ—, —(CRₐR_{b})ₘNRₐCO(CRₐR_{b})ₙ—, —(C RₐR_{b})ₘOOC(CRₐR_{b})ₙ—, —(CRₐR_{b})ₘCOO(CRₐR_{b})ₙ, —(CRₐR_{b})ₘNRₐCONR_{b}(CRₐR_{b})ₙ—, —(CRₐR_{b})ₘNRₐCOO(CRₐR_{b})ₙ—, —(CRₐR_{b})ₘOOCNRₐ(CRₐR_{b})ₙ—, where, m, n=0˜5 independently;

X is hydrogen, —CN, —F, —Cl, —Br, —NO₂, —CF₃, —CONRₐR_{b}, —NRₐCONRₐR_{b}, —NRₐCOR_{b}, —CORₐ, —SRₐ, —S(O)Rₐ, —S(O)₂Rₐ, —CH₂S(O)₂Rₐ, —SO₂NRₐR_{b}, CH₂SO₂NRₐR_{b}, C₁-C₅ alkyl, C₁-C₅ alkenyl, C₁-C₅ alkynyl, optionally substituted aryl, heteroaryl, cyclic alkyl or cyclic heteroalkyl wherein the substituents are independently represented by X;

Y is chosen from hydrogen, C₁-C₁₀ alkyl, C₁-C₁₀ alkenyl, C₁-C₁₀ alkynyl, aryl, heteroaryl, cyclic alkyl, cyclic heteroalkyl, heteroalkyl, haloalkyl, perhaloalkyl, —COR_{c}, —CONR_{c}, —SO₂Rₐ and —SO₂NRₐR_{b}, any of which may be optionally substituted with R_{d};

R₁, R₂, R₃ are substituents independently attached to A, B or cyclohexane ring which could be chosen from hydrogen, —CN, —F, —Cl, —Br, —NO₂, —OR_{c}, —NRₐR_{b}, —COR_{d}, —CONRₐR_{b}, C₁-C₅ alkyl and each A, B or cyclohexane ring can have above substituents from one up to four of them independently;

Rₐ and R_{b} is independently hydrogen, C₁-C₆ alkyl, or OR_{c}; R_{c} is hydrogen, C₁-C₁₀ alkyl, C₁-C₁₀ heteroalkyl; R_{d} has the formula of (W)ⱼ where j=0˜2 independently and W is independently chosen from hydrogen, —CN, —F, —Cl, —Br, —NO₂, —OR_{c}, —NRₐR_{b}, CORₐ, CONRₐR_{b}, NRₐCONRₐR_{b}, C₁-C₈ alkyl, C₃-C₈ cycloalkyl, C₂-C₈ alkenyl, C₂-C₈ alkynyl.

### DETAILED DESCRIPTION

This invention provides a compound of formula I, or a pharmaceutically acceptable salt, isomer or prodrug thereof:

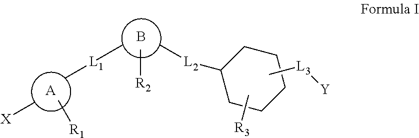

wherein,

A is 5˜6 membered aryl, heteroaryl, cyclic alkyl and cyclic heteroalkyl;

B is (C)ᵢ where i=0 or 1 independently, and C is independently chosen from 3˜6 membered aryl, heteroaryl, cyclic alkyl, and cyclic heteroalkyl;

L₁, L₂, L₃ are linkers having the formula of (Z)ₖ where k=0 or 1 independently and Z is independently chosen from —(CRₐR_{b})ₘ—, —(CRₐR_{b})ₘO(CRₐR_{b})ₙ—, —(CRₐR_{b})ₘCO(CRₐR_{b})ₙ—, —(CRₐR_{b})ₘS(CRₐR_{b})ₙ—, —(CRₐR_{b})ₘSO(CRₐR_{b})ₙ—, —(CRₐR_{b})ₘSO₂(CRₐR_{b})ₙ—, —(CRₐR_{b})ₘNRₐ(CRₐR_{b})ₙ—, —(CRₐR_{b})ₘCONRₐ(CRₐR_{b})ₙ—, —(CRₐR_{b})ₘNRₐCO(CRₐR_{b})ₙ—, —(CRₐR_{b})ₘOOC(CRₐR_{b})ₙ—, —(CRₐR_{b})ₘCOO(CRₐR_{b})ₙ, —(CRₐR_{b})ₘNRₐCONR_{b}(CRₐR_{b})ₙ—, —(CRₐR_{b})ₘNRₐCOO(CRₐR_{b})ₙ—, —(CRₐR_{b})ₘOOCNRₐ(CRₐR_{b})ₙ—,

where, m, n=0˜5 independently;

X is hydrogen, —CN, —F, —Cl, —Br, —NO₂, —CF₃, —ORₐ, —CONRₐR_{b}, —NRₐCONRₐR_{b}, —NRₐCOR_{b}, —CORₐ, —SRₐ, —S(O)Rₐ, —S(O)₂Rₐ, —CH₂S(O)₂Rₐ, —SO₂NRₐR_{b}, CH₂SO₂NRₐR_{b}, C₁-C₅ alkyl, C₁-C₅ alkenyl, C₁-C₅ alkynyl, optionally substituted aryl, heteroaryl, cyclic alkyl or cyclic heteroalkyl wherein the substituents are independently represented by X;

Y is chosen from hydrogen, C₁-C₁₀ alkyl, C₁-C₁₀ alkenyl, C₁-C₁₀ alkynyl, aryl, heteroaryl, cyclic alkyl, cyclic heteroalkyl, heteroalkyl, haloalkyl, perhaloalkyl, —COR_{c}, —CONR_{c}, —SO₂Rₐ and —SO₂NRₐR_{b}, any of which may be optionally substituted with R_{d};

R₁, R₂, R₃ are substituents independently attached to A, B or cyclohexane ring which could be chosen from hydrogen, —CN, —F, —Cl, —Br, —NO₂, —OR_{c}, —NRₐR_{b}, —COR_{d}, —CONRₐR_{b}, C₁-C₅ alkyl and each A, B or cyclohexane ring can have above substituents from one up to four of them independently;

Rₐ and R_{b} is independently hydrogen, C₁-C₆ alkyl, or OR_{c}; R_{c} is hydrogen, C₁-C₁₀ alkyl, C₁-C₁₀ heteroalkyl; R_{d} has the formula of (W)ⱼ where j=0˜2 independently and W is independently chosen from hydrogen, —CN, —F, —Cl, —Br, —NO₂, —OR_{c}, —NRₐR_{b}, —CORₐ, CONRₐR_{b}, NRₐCONRₐR_{b}, C₁-C₈ alkyl, C₃-C₈ cycloalkyl, C₂-C₈ alkenyl, C₂-C₈ alkynyl.

In addition, the invention provides a compound, or a pharmaceutically acceptable salt, isomer or prodrug thereof, wherein the compound is selected from the group consisting of:

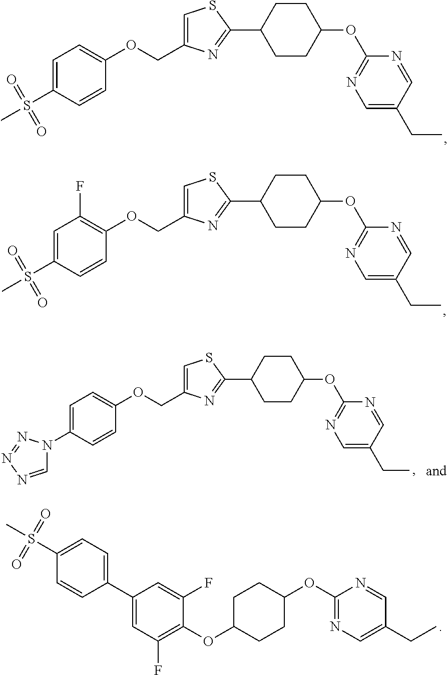

The invention also provides a pharmaceutical composition comprising the above compound, or a pharmaceutically acceptable salt, isomer or prodrug thereof and a pharmaceutically acceptable carrier or diluent. The pharmaceutical composition can further comprise another antidiabetic agent.

Moreover, the invention provides a method for treatment or delaying the progression or onset of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, or hypertension, which comprises administering to a mammal in need of treatment a therapeutically effective amount of the above compound, or a pharmaceutically acceptable salt, isomer or prodrug thereof:

The invention also provides a method for treatment of type 1 or type 2 diabetes mellitus, which comprises administering to a mammal in need of treatment a therapeutically effective amount of the above compound, or a pharmaceutically acceptable salt, isomer or prodrug thereof alone, or in combination with another antidiabetic agent, an agent for treating diabetic complications, an anti-obesity agent, an antihypertensive agent, an antiplatelet agent, an anti-atherosclerotic agent and/or a hypolipidemic agent.

Certain Chemical Terminology

Unless defined otherwise, all technical and scientific terms used herein have the same meaning as is commonly understood by one skilled in the art to which the claimed subject matter belongs.

Unless otherwise noted, the use of general chemical terms, such as though not limited to “alkyl” “amine” “aryl” are equivalent to their optionally substituted forms. For example, “alkyl,” as used herein, includes optionally substituted alkyl.

The compounds presented herein may possess one or more stereocenters and each center may exist in the R or S configuration, or combinations thereof. Likewise, the compounds presented herein may possess one or more double bonds and each may exist in the E (trans) or Z (cis) configuration, or combinations thereof. Presentation of one particular stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “optional or optionally” as used herein means that the subsequently described event or circumstance may occur or may not occur, and that the description includes instances where said event or circumstance occurs and instances in which it does not. For example, “optionally heteroaryl” means either “heteroaryl” or “substituted heteroaryl” as defined in the invention description. Further, we can choose up to 4 different species from the described set independently.

The term “aromatic” as used herein, refers to a planar, cyclic or polycyclic, ring moiety having a delocalized conjugated it system containing 4n+2 n electrons, where n is an integer (0, 1, 2, 3 so on). Aromatic rings can be formed by five, six, seven, eight, nine, or more than nine atoms. Aromatics can be optionally substituted and can be monocyclic or fused-ring polycyclic. The term aromatic encompasses both all carbon containing rings (e.g., phenyl) and those rings containing one or more heteroatoms (e.g., pyridine).

The term “aryl” as used herein, alone or in combination, refers to an aromatic group of 6 carbon atoms which does not contain heteroatoms and having a single ring (e.g., phenyl) or fused rings (e.g., naphthyl or anthryl). This also includes fused bicyclic ring system in which at least one of the fused rings is aromatic (e.g., tetrahydronaphtalene or tetrahydroisoquinoline).

The term “heteroaryl,” as used herein, alone or in combination, refers to a 5 or 6 membered unsaturated heteromonocyclic ring which contains at least one atom chosen from O, S, and N and this includes fused bicyclic ring system in which at least one of the fused rings is aromatic (e.g., tetrahydroquinazoline). Non-limiting examples of heteroaryl groups include thiazolyl, oxadiazolyl, imidazolyl, triazolyl, tetrazolyl, oxazolyl, isoxazolyl, pyrimidinyl, indolyl, pyrrolyl, pyrrolinyl, pyrazolyl, pyridyl, pyrazinyl, pyridazinyl, furyl, thienyl, benzotriazolyl, quinazolinyl, tetrahydropyridopyrimidinyl and the like.

The term “cyclic alkyl” as used herein, alone or in combination, refers to an optionally substituted, saturated, or partially unsaturated hydrocarbon. Cyclic alkyl includes from 3 (e.g. cyclopropyl) to 7 membered (e.g. cycloheptyl) single ring or fused ring (e.g., decahydronaphtalenyl). And fused ring includes a fused bridged ring (e.g., bicyclo[3,2,1]octanyl).

The term “cyclic heteroalkyl” as used herein, alone or in combination, refers to a stable saturated or partially unsaturated monocyclic or bicyclic group containing at least one heteroatom chosen from O, S, N wherein the nitrogen and sulfur atoms may optionally be oxidized. Heterocycles can be optionally substituted. Heterocycles includes from 3 (e.g., aziridnyl) to 7 membered single ring or fused ring (e.g. azabicyclo[3,2,1]octanyl). And fused ring includes a fused bridged ring (e.g. azabicyclo[3.2.1]octanyl). A non-limiting example of “cyclic alkyl” includes azinyl, azetidinyl, oxetanyl, thietanyl, piperidinyl, oxepanyl, piperazinyl, thiepanyl, pyrrolidinyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, and quinolizinyl and the like.

As used herein, C₁-Cₙ indicates that there are one to n carbon atoms in the moiety, i.e. groups containing 1 carbon atom, 2 carbon atoms, 3 carbon atoms or n carbon atoms. For further explanation, it means C₁, C₂, C₃ . . . Cₙ₋₁, Cₙ. Thus, by way of example only, “C₁-C₄ alkyl” indicates that there are one to four carbon atoms in the alkyl group and this includes cycloalkyl group, i.e., the alkyl group is selected from among methyl, ethyl, propyl, iso-propyl, n-butyl, isobutyl, sec-butyl, t-butyl, cyclopropyl; or cyclobutyl.

The terms “heteroatom” or “hetero” as used herein, alone or in combination, refer to an atom other than carbon and hydrogen. Heteroatoms are independently selected from among oxygen, nitrogen, sulfur, phosphorous, silicon, selenium and tin but are not limited to these atoms. In an embodiment in which two or more heteroatoms are present, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain saturated hydrocarbon. Non-limiting examples include, methyl, ethyl, n-propyl, isopropyl, 2-methyl-1-propyl, 2-methyl-2-propyl, 2-methyl-1-butyl, 3-methyl-1-butyl, 2-methyl-3-butyl; 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neo-pentyl, tert-amyl and hexyl, and longer alkyl groups, such as heptyl, octyl and the like.

The term “alkenyl” as used herein, alone or in combination, refers to an optionally substituted straight-chain, or optionally substituted branched-chain hydrocarbon monoradical having one or more carbon-carbon double bonds and having from two to about ten carbon atoms. The group may be in either the cis or trans conformation about the double bond(s), and should be understood to include both isomers. A non-limiting examples are ethenyl (CH═CH₂), 1-propenyl (CH₂CH═CH₂), isopropenyl [C(CH₃)═CH₂], butenyl, 1,3-butadienyl and the like.

The term “alkynyl” as used herein, alone or in combination, refers to an optionally substituted straight-chain or optionally substituted branched-chain hydrocarbon monoradical having one or more carbon-carbon triple bonds and having from two to about ten carbon atoms. Non-limiting examples include, ethynyl, 2-propynyl, 2-butynyl, 1,3-butadiynyl and the like.

The terms “heteroalkyl”, “heteroalkenyl” and “heteroalkynyl” as used herein, alone or in combination, refer to optionally substituted alkyl, alkenyl and alkynyl structures respectively, as described above, in which one or more of the skeletal chain carbon atoms (and any associated hydrogen atoms, as appropriate) are each independently replaced with heteroatoms (e.g. —CH₂OH, —OCH₂CH₃, —CH₂NH₂, —NHCH₃).

The terms “haloalkyl”, “haloalkenyl” and “haloalkynyl” as used herein, alone or in combination, refer to optionally substituted alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms may be replaced with halogen atoms that are the same as each another (e.g. difluoromethyl); in other embodiments two or more hydrogen atoms may be replaced with halogen atoms that are not all the same as each other (e.g. 1-chloro-1-fluoro-1-iodoethyl).

The term “perhalo” as used herein, alone or in combination, refers to groups in which all of the hydrogen atoms are replaced by fluorines, chlorines, bromines, iodines, or combinations thereof. A non-limiting example of a perhaloalkyl group is a trifluoromethyl. A non-limiting example of a perhaloalkenyl group is trichloroethenyl. A non-limiting example of a perhaloalkynyl group is tribro-mopropynyl.

Certain Pharmaceutical Terminology

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “administer” “administering”, “administration” and the like, as used herein, refer to the methods that may be used to enable delivery of compounds or compositions to the desired site of biological action. These methods include, but are not limited to, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “agonist,” as used herein, refers to a molecule such as a compound, a drug, an enzyme activator or a hormone modulator which enhances the activity of another molecule or the activity of a receptor site.

The term “pharmaceutically acceptable salt” as used herein, refers to pharmaceutically acceptable salts that retain the biological effectiveness of the free acids and bases of the specified compound derived from a variety of organic and inorganic counter ions well known in the art. These include, by way example only, sodium, potassium, calcium, magnesium, ammonium, tetraalkylaminonium, acetate, acrylate, adipate, alginate, aspartate, benzoate, benzenesulfonate, bisulfate, bisulfite, bromide, iodide, butyrate, butyn-1,4-dioate, camphorate, camphorsulfonate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, isobutyrate, lactate, maleate, malonate, methanesulfonate, mandelate. metaphosphate, methoxybenzoate, methyl benzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts. Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, IV′ (C₁_{_{—}}₄ alkyl)₄, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization.

The term “solvate” as used herein refers to a combination of a compound of this invention with a solvent molecule formed by solvation. In some situations, the solvate refers to a hydrate, i.e., the solvent molecule is a water molecule, the combination of a compound of this invention and water forms a hydrate.

The term “pharmaceutically acceptable derivative or prodrug” as used herein, refers to any derivative of a compound of the embodiments that is capable of directly or indirectly providing a compound of this invention or an active metabolite or residue thereof upon administration to a recipient. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system). A non-limiting examples are esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters.

The terms “pharmaceutical combination or combinational therapy” as used herein, refer to a pharmaceutical therapy resulting from mixing or combining more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

Methods for synthesizing the compounds described herein are provided. In some embodiments, the compounds described herein can be prepared by the methods described below. The procedures and examples below are intended to illustrate those methods. Neither the procedures nor the examples should be construed as limiting the invention in any way. Compounds described herein may also be synthesized using standard synthetic techniques known to those of skill in the art or using methods known in the art in combination with methods described herein.

### EXAMPLES

NMR spectra were recorded using commercially available NMR solvents (e.g. CDCl₃, CD₃OD or DMSO-d₆) in 5-mm o.d. tubes (Norell, Inc. 507-HP) at 30° C. and were collected on Varian VNMRS-400 at 400 MHz for ¹H. The chemical shifts (δ) are relative to tetramethylsilane (TMS, =0.00 ppm) and expressed in ppm.

LC/MS was taken on Ion-trap Mass Spectrometer on FINNIGAN Thermo LCQ Advantage MAX, Agilent LC 1200 series (Column: Agilent Extend (C18, Ø4.6×50 mm, 3 μm, 120 Å, 40° C.) operating in ESI(+) ionization mode; flow rate=1.0 mL/min. Mobile phase=0.01% heptafluorobutric acid (HFBA) and 10% isopropyl alcohol (IPA) in water or CH₃CN.

### Example 1

### Preparation of 2-(4-(4-((4-(methylsulfonyl)phenoxy)methyl)-1H-1,2,3-triazol-1-yl)cyclohexyloxy)pyrimidine

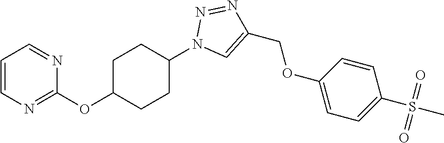

### Step 1. Preparation of 1,4-dioxaspiro[4.5]decan-8-ol.

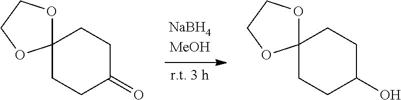

To a solution of 1,4-dioxaspiro[4.5]decan-8-one (15.0 g, 96 mmol) in MeOH (240 mL) was added NaBH₄ (4.00 g, 106 mmol) in small portions at 0° C. After being stirred for 3 hours at room temperature, the reaction mixture was concentrated in vacuo and brine was added. The mixture was extracted with EtOAc, dried over anhydrous Na₂SO₄, filtered and passed through a short pad of silica gel and concentrated in vacuo to give the desired product (14.2 g, 93%) as a colorless oil. ¹H-NMR (400 MHz, CDCl₃) δ 1.54-1.67 (4H, m), 1.79-1.91 (5H, m), 3.77-3.83 (1H, m), 3.91-3.98 (4H, m).

### Step 2. Preparation of 2-(1,4-dioxaspiro[4.5]decan-8-yloxy)pyrimidine

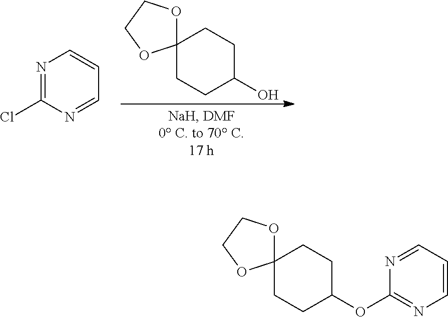

To a stirred solution of 1,4-dioxaspiro[4.5]decan-8-ol (4.00 g, 25.3 mmol) in dry DMF (75.0 mL) was added sodium hydride (1.40 g, 32.1 mmol. 55 wt % dispersion in mineral oil) at 0° C. After being stirred for 30 minutes at room temperature, 2-chloropyrimidine (3) (3.20 g, 27.9 mmol) was added to the mixture at 0° C. The mixture was heated to 70° C. for 17 hours and cooled to room temperature. The reaction mixture was quenched with ice-water and extracted with EtOAc. The organic layer was washed with brine, dried over anhydrous Na₂SO₄, filtered and concentrated in vacuo. The residue was purified by column chromatography on SiO₂ (Hexanes:EtOAc=1:1) to give the desired product (5.80 g, 97%) as a yellow oil. ¹H-NMR (400 MHz, CDCl₃) δ 1.65-1.71 (2H, m), 1.91-2.07 (6H, m), 3.94-4.01 (4H, m), 5.11-5.16 (1H, m), 6.90 (1H, t, J=4.8 Hz), 8.50 (1H, d, J=4.4 Hz).

### Step 3. Preparation of 4-(pyrimidin-2-yloxy)cyclohexanone

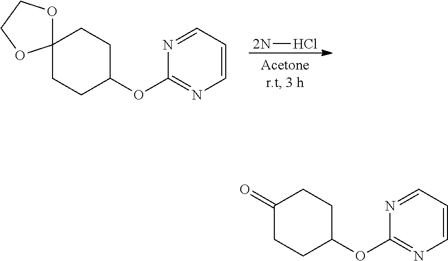

To a solution of 2-(1,4-dioxaspiro[4.5]decan-8-yloxy)pyrimidine (5.80 g, 24.6 mmol) in acetone (50.0 mL) was added aq. 2N hydrogen chloride (37.0 mL, 74.0 mmol) at 0° C. After being stirred for 3 hours at room temperature, the reaction mixture was concentrated in vacuo. The residue was diluted with Et₂O, washed with water and brine, dried over Na₂SO₄, filtered and concentrated in vacuo to give the desired product (2.67 g, 57%) as a white solid. ¹H-NMR (400 MHz, CDCl₃) δ 2.14-2.23 (2H, m), 2.30-2.43 (4H, m), 2.69-2.77 (2H, m), 5.44-5.47 (1H, m), 6.97 (1H, t, J=4.4 Hz), 8.54 (2H, d, J=4.8 Hz).

### Step 4. Preparation of 4-(pyrimidin-2-yloxy)cyclohexanol

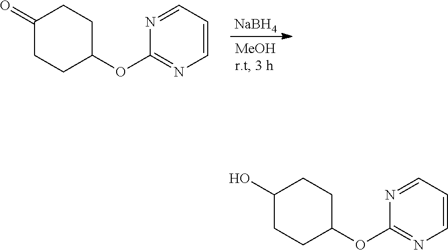

To a solution of 4-(pyrimidin-2-yloxy)cyclohexanone (1.00 g, 5.20 mmol) in MeOH (20.0 mL) was added NaBH₄ (240 mg, 6.34 mmol) in small portions at 0° C. After being stirred for 3 hours at room temperature, the reaction mixture was concentrated in vacuo and brine was added. The mixture was extracted with EtOAc, dried over anhydrous Na₂SO₄, filtered and passed through a short pad of silica gel and concentrated in vacuo to give the desired crude product (780 mg, 77%) as a colorless oil, which was used without further purification. LC-MS Calcd. 194.11, Found 194.86.

### Step 5. Preparation of 4-(pyrimidin-2-yloxy)cyclohexyl 4-methylbenzenesulfonate

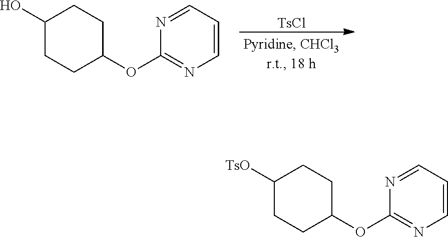

To a stirred solution of 4-(pyrimidin-2-yloxy)cyclohexanol (780 mg, 4.02 mmol) in CHCl₃ (8.00 mL) was added pyridine (1.00 mL, 12.4 mmol) and TsCl (840 mg, 4.41 mmol) at 0° C. After being stirred for 18 hours at room temperature, the reaction mixture was diluted with water and extracted with EtOAc. The organic layer was washed with brine, dried over anhydrous Na₂SO₄, filtered and concentrated in vacuo. The residue was purified by column chromatography on SiO₂ (Hexanes:EtOAc=5:1 to 1:1) and recrystallized from hexanes/EtOAc mixture to give the desired product (776 mg, 43% for two steps) as a white solid. ¹H-NMR (400 MHz, CDCl₃) δ 1.72-1.82 (4H, m), 2.00-2.10 (4H, m), 2.45 (3H, d, J=2.8 Hz), 4.65-4.69 (1H, m), 5.04-5.10 (1H, m), 6.89-6.91 (1H, m), 7.32-7.36 (2H, m), 7.81-7.83 (2H, m), 8.47-8.49 (2H, m).

### Step 6. Preparation of 2-(4-azidocyclohexyloxy)pyrimidine

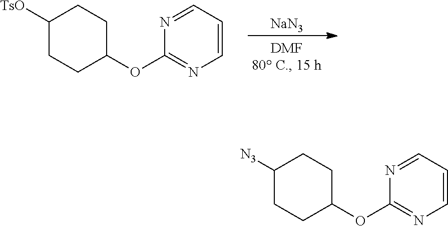

To a solution of 4-(pyrimidin-2-yloxy)cyclohexyl-4-methylbenzenesulfonate (785 mg, 2.25 mmol) in dry DMF (8.00 mL) was added sodium azide (290 mg, 4.46 mmol) at room temperature. After being stirred for 15 hours at 80° C. the reaction mixture was diluted with water and extracted with EtOAc. The organic layer was washed with brine, dried over anhydrous Na₂SO₄, filtered and concentrated in vacuo. The residue was purified by column chromatography on SiO₂ (EtOAc only) to give the desired product (490 mg, 99%) as a yellow oil. ¹H-NMR (400 MHz, CDCl₃) δ 1.53-1.71 (2H, m), 1.74-1.83 (2H, m). 1.88-2.20 (4H, m), 3.52-3.57 (1H, m), 5.02-5.15 (1H, m), 6.91 (1H, t, J=4.8 Hz), 8.50 (1H, d, J=4.4 Hz).

### Step 7. Preparation of 1-(methylsulfonyl)-4-(prop-2-ynyloxy)benzene

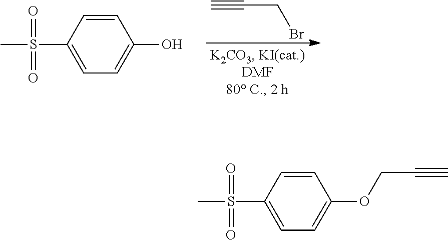

To a solution of 4-(methylsulfonyl)phenol (200 mg, 1.16 mmol) in dry DMF (2.00 mL) was added potassium carbonate (321 mg, 2.32 mmol) potassium iodide (12.0 mg, 0.070 mmol), and propagyl bromide (0.120 mL, 1.59 mmol) at room temperature. After being stirred for 2 hours at 80° C., the reaction mixture was diluted with water and extracted with EtOAc. The organic layer was washed with brine, dried over anhydrous Na₂SO₄, filtered and concentrated in vacuo. The residue was purified by column chromatography on SiO₂ (Hexanes:EtOAc=1:1) to give the desired product (230 mg, 94%) as a pale yellow solid. ¹H-NMR (400 MHz, CDCl₃) δ 2.58 (1H, s), 2.58 (3H, s), 4.78 (2H, s), 7.12 (2H, d, J=8.8 Hz), 7.90 (2H, d, J=8.8 Hz).

### Step 8. Preparation of 2-(4-(4-((4-(methylsulfonyl)phenoxy)methyl)-1H-1,2,3-triazol-1-yl)cyclohexyloxy)pyrimidine

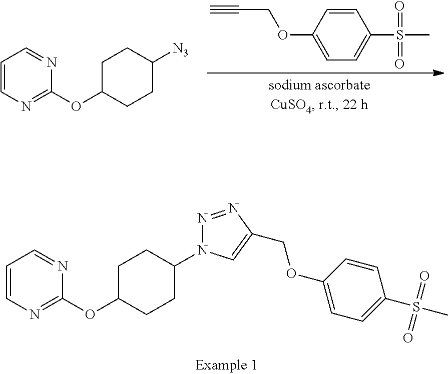

To a solution of 2-(4-azidocyclohexyloxy)pyrimidine (100 mg, 0.456 mmol) in water (0.800 mL) was added 1-(methylsulfonyl)-4-(prop-2-ynyloxy)benzene (96.0 mg, 0.456 mmol), 1.0 M aq. sodium ascorbate (0.100 mL, 0.100 mmol), and 1.0 M aq. copper sulfate (0.100 mL, 0.100 mmol) at room temperature. After being stirred for 20 hours at room temperature, the reaction mixture was diluted with water and extracted with EtOAc. The organic layer was washed with brine, dried over anhydrous Na₂SO₄, filtered and concentrated in vacuo. The residue was purified by recrystallization from hexanes/EtOAc mixture to give the desired product (145 mg, 74%) as a white solid. ¹H-NMR (400 MHZ CDCl₃) δ 1.76-1.88 (2H, m), 2.01-2.16 (2H, m), 2.22-2.43 (4H, m), 3.04 (3H, s), 4.54-4.70 (1H, m), 5.08-5.39 (1H, m), 5.30 (2H, s), 6.93-6.97 (1H, m), 7.15 (2H, d, J=8.0 Hz), 7.66 and 7.74 (1H, s), 7.89 (2H, d, J=8.0 Hz), 8.51-8.53 (2H, m). LC-MS Calcd. 429.15 Found 430.1 [M+H]⁺.

### Example 2

### Preparation of 5-methyl-2-(4-(4-((4-(methylsulfonyl)phenoxy)methyl)-1H-1,2,3-triazol-1-yl)cyclohexyloxy)pyrimidine

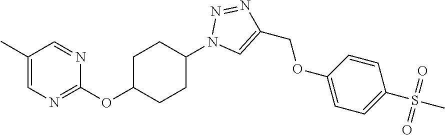

### Step 1. Preparation of 2-chloro-5-methylpyrimidine

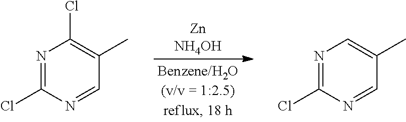

To a stirred solution of 2,4-dichloro-5-methylpyrimidine (4.00 g, 24.5 mmol) in a mixture of benzene (16.0 mL) and H₂O (40.0 mL) was added zinc powder (4.81 g, 73.6 mmol) and ammonia water (8.80 mL, 24.5 mmol) at room temperature. After heating at reflux for 18 hours, the reaction mixture was cooled and filtered through a pad of Celite and the reaction mixture was extracted with Et₂O, washed with brine, dried over anhydrous Na₂SO₄, filtered and concentrated in vacuo. The residue was purified by column chromatography on SiO₂ (Hexanes:EtOAc=1:1) to give the desired product (2.44 g, 77%) as a yellow oil. ¹H-NMR (400 MHz, CDCl₃) δ 2.33 (3H, s), 8.47 (2H, s).

### Step 2. Preparation of 2-(1,4-dioxaspiro[4.5]decan-8-yloxy)-5-methylpyrimidine

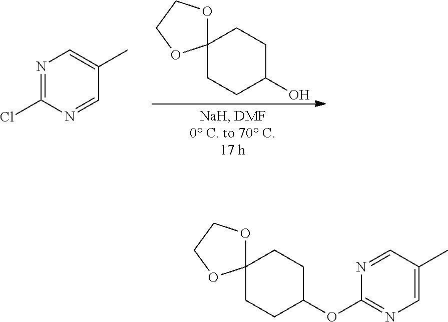

According to the procedure of step 2 in Example 1, the desired product was obtained. ¹H-NMR (400 MHz, CDCl₃) δ 1.61-1.70 (2H, m), 1.91-2.05 (6H, m), 2.22 (3H, s), 3.94-3.99 (4H, m), 5.07-5.10 (1H, m), 8.47 (2H, s).

### Step 3. Preparation of 4-(5-methylpyrimidin-2-yloxy)cyclohexanone

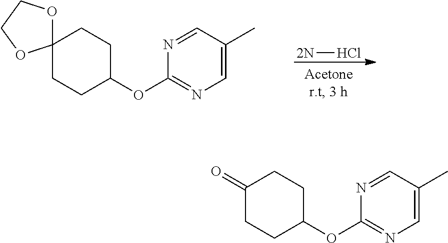

According to the procedure of step 3 in Example 1, the desired product was obtained. ¹H-NMR (400 MHz, CDCl₃) δ 2.12-2.22 (2H, m), 2.25 (3H, s), 2.29-2.42 (4H, m), 2.69-2.77 (2H, m), 5.38-5.42 (1H, m), 8.35 (2H, s).

### Step 4. Preparation of 4-(5-methylpyrimidin-2-yloxy)cyclohexanol

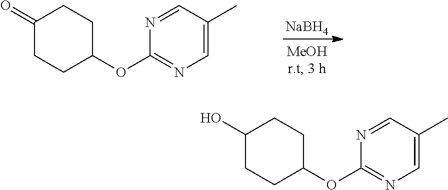

According to the procedure of step 4 in Example 1, the desired product was obtained. LC-MS Calcd. 208.12, Found 208.84.

### Step 5. Preparation of 4-(5-methylpyrimidin-2-yloxy)cyclohexyl 4-methylbenzenesulfonate

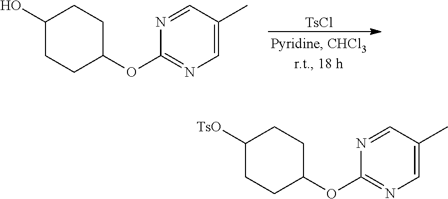

According to the procedure of step 5 in Example 1, the desired product 15 was obtained. ¹H-NMR (400 MHz, CDCl₃) δ 1.68-1.80 (4H, m), 1.96-2.09 (4H, m), 2.21 (3H, s). 2.44-2.45 (3H, m), 4.64-4.70 (1H, m), 4.97-5.04 (1H, m), 7.32-7.36 (2H, m), 7.81 (2H, dd, J=8.4, 2.4 Hz), 8.29 (2H, s).

### Step 6. Preparation of 2-(4-azidocyclohexyloxy)-5-methylpyrimidine

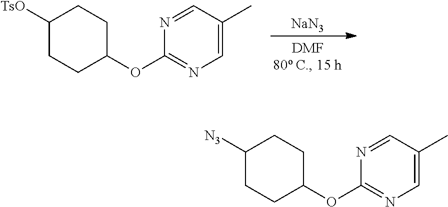

According to the procedure of step 6 in Example 1, the desired product was obtained. ¹H-NMR (400 MHz, CDCl₃) δ 1.52-1.82 (4H, m). 1.89-2.20 (4H, m), 2.17 (3H, s), 3.51-3.56 (1H, m), 4.97-5.10 (1H, m), 8.31 (2H, s).

### Step 7 Preparation of 5-methyl-2-(4-(4-((4-(methylsulfonyl)phenoxy)methyl)-1H-1,2,3-triazol-1-yl)cyclohexyloxy)pyrimidine

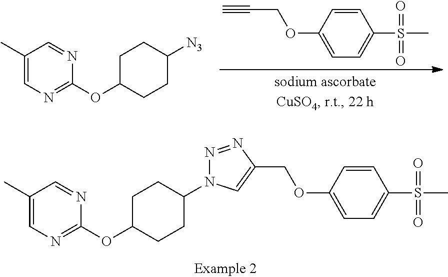

According to the procedure of step 8 in Example 1, the desired compound, Example 2 was obtained. ¹H-NMR (400 MHz, CDCl₃,) δ 1.77-1.86 (2H, m), 2.03-2.13 (2H, m), 2.23 (3H, s), 2.27-2.40 (4H, m), 3.04 (3H, s), 4.55-4.68 (1H, m), 5.05-5.34 (1H, m), 5.29 (2H, s), 7.15 (2H, d, J=6.8 Hz), 7.65 and 7.73 (1H, s), 7.89 (2H, d, J=7.2 Hz). 8.33-8.34 (2H, m). LC-MS Calcd. 443.16 Found 444.1 [M+H]⁺.

### Example 3

### Preparation of 5-ethyl-2-(4-(4-((4-(methylsulfonyl)phenoxy)methyl)-1H-1,2,3-triazol-1-yl)cyclohexyloxy)pyrimidine

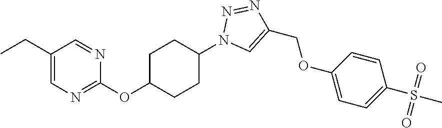

### Step 1. Preparation of 2-(1,4-dioxaspiro[4.5]decan-8-yloxy)-5-ethylpyrimidine

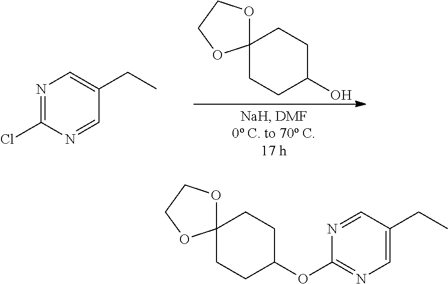

According to the procedure of step 2 in Example 1, the desired product was obtained. ¹H-NMR (400 MHz, CDCl₃) δ 1.24 (3H, t, J=7.6 Hz), 1.64-1.70 (2H, m), 1.92-2.06 (6H, m), 2.57 (2H, q, J=7.6 Hz), 3.93-4.10 (4H, m), 5.06-5.12 m), 8.33 (2H, s).

### Step 2. Preparation of 4-(5-ethylpyrimidin-2-yloxy)cyclohexanone

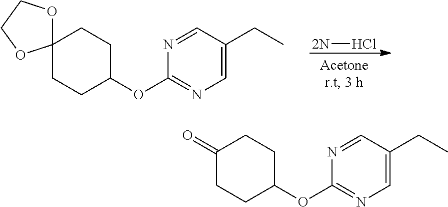

According to the procedure of step 3 in Example 1, the desired product was obtained. ¹H-NMR (400 MHz, CDCl₃) δ 1.26 (3H, t, J=7.6 Hz), 2.13-2.21 (2H, m), 2.29-2.42 (4H, m), 2.60 (2H, q, J=7.6 Hz), 2.69-2.77 (2H, m), 5.40-5.41 (1H, m), 8.37 (2H, s).

### Step 3. Preparation of 4-(5-ethylpyrimidin-2-yloxy)cyclohexanol

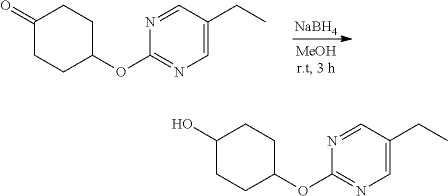

According to the procedure of step 4 in Example 1, the desired product was obtained. LC-MS Calcd.: 222.14; MS Found: 222.84.

### Step 4. Preparation of 4-(5-ethylpyrimidin-2-yloxy)cyclohexyl 4-methylbenzenesulfonate

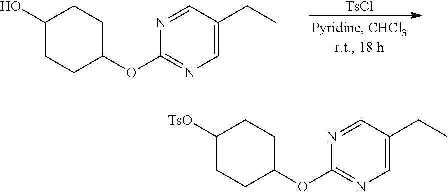

According to the procedure of step 5 in Example 1, the desired product was obtained. ¹H-NMR (400 MHz, CDCl₃) 8, 1.22 (3H, t, J=7.6 Hz), 1.66-1.81 (4H, m), 1.97-2.09 (4H, m), 2.44-2.45 (3H, m), 2.56 (2H, q, J=7.6 Hz), 4.64-4.71 (1H, m), 4.97-5.06 (1H, m), 7.32-7.36 (2H, m), 7.81 (2H, d, J=8.4, 2.0 Hz), 8.31 (2H, s).

### Step 5. Preparation of 2-(4-azidocyclohexyloxy)-5-ethylpyrimidine

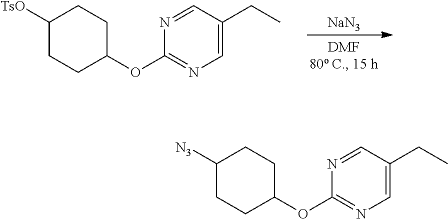

According to the procedure of step 6 in Example 1, the desired product was obtained ¹H-NMR (400 MHz, CDCl₁) δ 1.25 (3H, t, J=7.6 Hz), 1.53-1.83 (4H, m), 1.88-2.19 (4H, m), 2.57 (2H, q, J=7.6 Hz), 3.51-3.54 (1H, m), 4.98-5.11 (1H, m), 8.33 (2H, s).

### Step 6. Preparation of 5-ethyl-2-(4-(4-((4-(methylsulfonyl)phenoxy)methyl)-1H-1,2,3-triazol-1-yl)cyclohexyloxy)pyrimidine

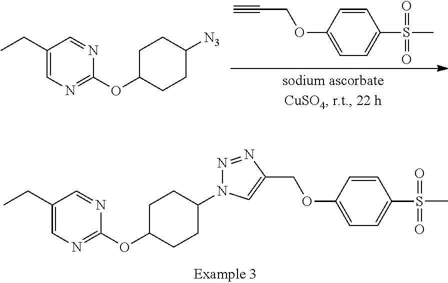

According to the procedure of step 8 in Example 1, the desired product was obtained. ¹H-NMR (400 MHz, CDCl₃) δ 1.26 (3H, t, J=7.6 Hz), 1.75-1.87 (2H, m), 2.01-2.13 (2H, m), 2.22-2.39 (4H, m), 2.59 (2H, q, J=7.6 Hz), 3.04 (3H, s), 4.54-4.71 (1H, m), 5.04-5.34 (1H, m), 5.29 (2H, s), 7.15 (2H, d, J=8.4 Hz), 7.66 and 7.73 (1H, s), 7.89 (2H, d, J=8.0 Hz), 8.35-8.36 (2H, m). LC-MS Calcd. 457.18 Found 458.1 [M+H]⁺.

### Example 4

### Preparation of 2-(4-(5-ethylpyrimidin-2-yloxy)cyclohexyl)-4-((4-(methylsulfonyl)phenoxy)methyl)thiazole

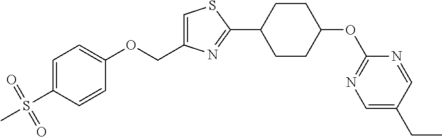

### Step 1. Preparation of 4-(5-ethylpyrimidin-2-yloxy)cyclohexanecarbonitrile

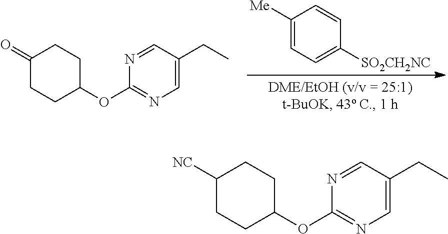

To a stirred solution of 4-(5-ethylpyrimidin-2-yloxy)cyclohexanone (4.30 g, 19.5 mmol) and 1-(isocyanomethylsulfonyl)-4-methylbenzene (5.00 g, 25.6 mmol) in a mixture of DME (40.0 mL) and EtOH (1.60 mL) was added t-BuOK (4.50 g, 40.1 mmol) in small portions at 0° C. After being stirred for 30 minutes at room temperature and then 1 hour at 40˜43° C., the suspension thus obtained was cooled to room temperature. The precipitate was washed with DME and removed. The combined solutions were concentrated in vacuo, and the residue was purified by column chromatography on SiO₂ (Hexanes:EtOAc=1:1 to EtOAc) to give the desired product (3.50 g, 78%) as a yellow wax. ¹H-NMR (400 MHz, CDCl₃) δ 1.25 (3H, t, J=6.0 Hz), 1.76-1.91 (4H, m), 1.99-2.25 (4H, m), 2.59 (2H, q, J=6.0 Hz), 2.70-2.75 (1H, m), 5.05-5.10 (1H, m), 8.33 (2H, s).

### Step 2. Preparation of 4-(5-ethylpyrimidin-2-yloxy)cyclohexanecarbothioamide

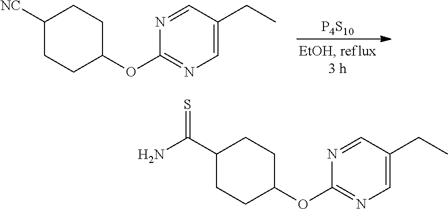

A solution of phosphorous pentasulfide (13.0 g, 29.2 mmol) in absolute EtOH (20.0 mL) was stirred for 1 hour at room temperature. 4-(5-Ethylpyrimidin-2-yloxy)cyclohexanecarbonitrile (3.50 g, 15.1 mmol) in EtOH (10.0 mL) was added to the above reaction mixture at room temperature. After being heated at reflux for 3 hours, the reaction mixture was diluted with water and extracted with CHCl₃. The organic layer was washed with brine, dried over anhydrous Na₂SO₄, filtered and concentrated in vacuo to give the desired product which was used without further purification. ¹H-NMR (400 MHz, CDCl₃) δ 1.26 (3H, t, J=6.0 Hz), 1.51-1.71 (2H, m), 1.80-2.06 (4H, m), 2.08-2.32 (2H, m), 2.59 (2H, q, J=6.0 Hz), 2.84-2.90 (1H, m), 4.94-5.02 (1H, m), 6.96-7.15 (1H, m), 7.49-7.55 (1H, m), 8.33-8.37 (2H, m).

### Step 3. Preparation of 4-(chloromethyl)-2-(4-(5-ethylpyrimidin-2-yloxy)cyclohexyl) thiazole

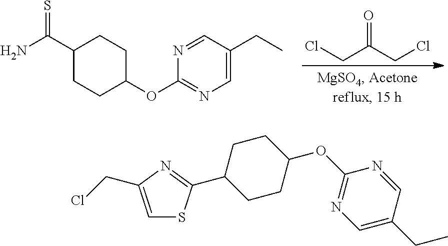

To a stirred solution of 4-(5-ethylpyrimidin-2-yloxy)cyclohexanecarbothioamide (1.80 g, 6.78 mmol) in acetone (25.0 mL) was added 1,3-dichloroacetone (1.20 g, 9.45 mmol) and magnesium sulfate (2.00 g, 16.6 mmol) at room temperature. The mixture was heated at reflux for 15 hours and diluted with water. The mixture was extracted with EtOAc. The combined organic layers were washed with brine, dried over anhydrous Na₂SO₄, filtered and concentrated in vacuo. The residue was purified by column chromatography on SiO₂ (Hexanes:EtOAc=1:1) to give the desired product (1.00 g, 20% for two steps) as a yellow oil. ¹H-NMR (400 MHz, CDCl₃) δ 1.25 (3H, t, J=7.6 Hz), 1.65-1.82 (3H, m), 2.00-2.14 (2H, m), 2.21-2.55 (3H, m), 2.58 (2H, q, J=7.6 Hz), 3.06-3.19 (1H, m), 4.67 (2H, s), 4.97-5.31 (1H, m), 7.18 (1H, s), 8.34 (2H, d, J=3.6 Hz).

### Step 4. Preparation of 2-(4-(5-ethylpyrimidin-2-yloxy)cyclohexyl)-4-((4-(methylsulfonyl)phenoxy)methyl)thiazole

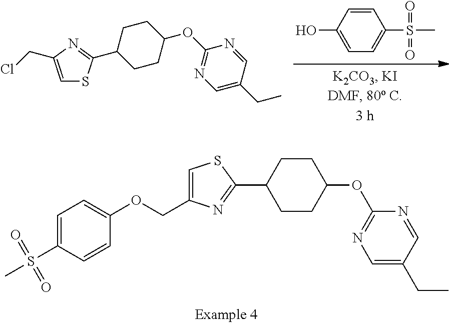

To a stirred solution of 4-(chloromethyl)-2-(4-(5-ethylpyrimidin-2-yloxy)cyclohexyl)thiazole (70.0 mg, 0.207 mmol) in dry DMF (1.30 mL) was added 4-(methylsulfonyl)phenol (36.0 mg, 0.207 mmol), potassium carbonate (57.0 mg, 0.414 mmol) and potassium iodide (10.0 mg, 0.060 mmol) at room temperature. After being stirred for 3 hours at 80° C., the reaction mixture was diluted with water and extracted with EtOAc. The organic layer was washed with brine, dried over anhydrous Na₂SO₄, filtered and concentrated in vacuo. The residue was purified by recrystallization from hexanes/EtOAc mixture to give the desired product (84.0 mg, 86%) as a white solid. ¹H-NMR (400 MHz, CDCl₃) δ 1.25 (3H, t, J=7.6 Hz), 1.69-1.83 (3H, m), 2.01-2.21 (2H, m), 2.22-2.35 (3H, m), 2.58 (2H, q, J=7.6 Hz), 3.04 (3H, s), 3.07-3.19 (1H, m), 5.00 and 5.31 (1H, m), 5.24 (2H, d, J=2.0 Hz), 7.13 (2H, d, J=8.8 Hz), 7.21 (1H, s), 7.88 (2H, dd, J=8.8, 2.4 Hz), 8.34 (2H, d, J=3.2 Hz). LC-MS Calcd. 473.14 Found 474.1 [M+H]⁺.

### Example 5

### Cis, Trans

Cis-(2-((1s,4s)-4-(5-ethylpyrimidin-2-yloxy)cyclohexyl)-4-((2-fluoro-4-(methylsulfonyl)phenoxy)methyl)thiazole)

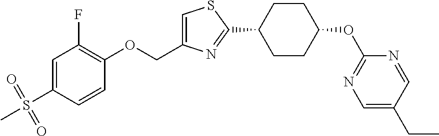

Trans-(2-((1r,4r)-4-(5-ethylpyrimidin-2-yloxy)cyclohexyl)-4-((2-fluoro-4-methylsulfonyl)phenoxy)methyl)thiazole)

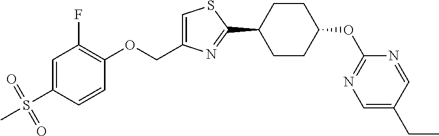

### Step 1. Preparation of (2-(4-(5-ethylpyrimidin-2-yloxy)cyclohexyl)-4-((2-fluoro-4-(methylsulfonyl)phenoxy)methyl)thiazole)

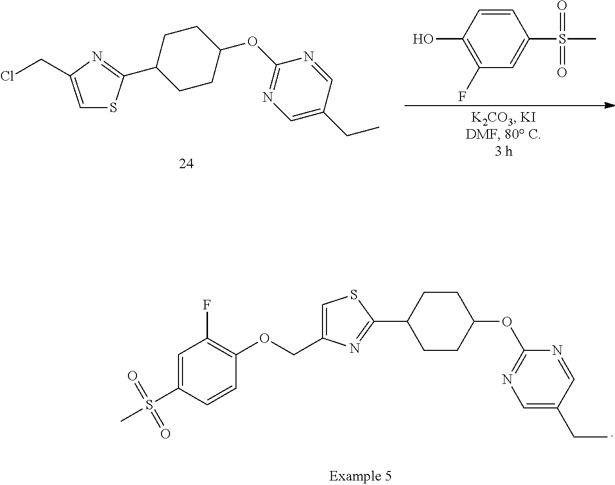

According to the procedure of step 4 in Example 4, the desired product was obtained. ¹H-NMR (400 MHz, CDCl₃) δ 1.25 (3H, t, J=7.2 Hz), 1.69-1.83 (3H, m), 2.01-2.14 (2H, m), 2.21-2.35 (3H, m), 2.58 (2H, q, J=7.2 Hz), 3.05 (3H, s), 3.08-3.17 (1H, m), 5.00 and 5.30 (1H, m), 5.32 (2H, s), 7.24-7.28 (2H, m), 7.65-7.70 (2H, m), 8.34 (2H, d, J=2.8 Hz). LC-MS Calcd. 491.13 Found 492.0 [M+H]⁺.

### Step 2. Preparation of Example 5—cis & trans

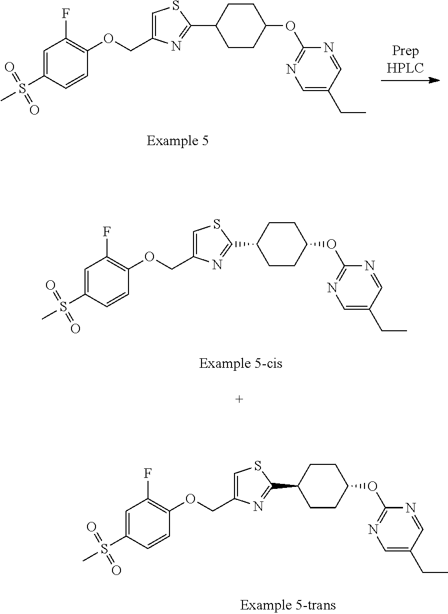

Two isomers in Example 5 (140 mg, 0.302 mmol) were isolated by preparative HPLC through Waters Deltaprep 300 series (Column: Agilent Extend (C18, Ø30×L150 mm, 5 μm) operating in ESI(+) ionization mode (flow rate=42.53 mL/min. Mobile phase=50% isocratic in water or CH₃CN) to give the desired Example 5—cis (65.0 mg, retention time 9.72 min.) & Example 5—trans (80.0 mg, retention time 8.85 min.) as a white solid each.

Example 5—Cis (2-((1s,4s)-4-(5-ethylpyrimidin-2-yloxy)cyclohexyl)-4-((2-fluoro-4-(methylsulfonyl)phenoxy)methyl)thiazole) ¹H-NMR (400 MHz, CDCl₃) δ 1.25 (3H, t, J=7.6 Hz), 1.79-1.83 (2H, m), 2.01-2.25 (6H, m), 2.58 (2H, q, J=7.6 Hz), 3.05 (3H, s), 3.14-3.15 (1H, m), 5.29-5.30 (1H, m). 5.32 (2H, s), 7.24-7.28 (1H, m), 7.65-7.71 (2H, m), 8.35 (2H, s), LC-MS Calcd. 491.13 Found 492.2 [M+H]⁺.

Example 5—Trans (2-((1r,4r)-4-(5-ethylpyrimidin-2-yloxy)cyclohexyl)-4-((2-fluoro-4-(methylsulfonyl)phenoxy)methyl)thiazole) ¹H-NMR (400 MHz, CDCl₃,) δ 1.25 (3H, t, J=7.6 Hz), 1.69-1.82 (4H, m), 2.28-2.35 (4H, m), 2.58 (2H, q, J=7.6 Hz), 3.05 (3H, s), 3.07-3.12 (1H, m), 5.00-5.05 (1H, m), 5.30 (2H, s), 7.24-7.27 (1H, m), 7.66-7.71 (2H, m), 8.34 (2H, s). LC-MS Calcd.: 491.13; MS Found: 492.0 [M+H]⁺.

### Example 6

### Cis, Trans

### Cis-(4-((4-(1H-tetrazol-1-yl)phenoxy)methyl)-2-((1s,4s)-4-(5-ethylpyrimidin-2-yloxy)cyclohexyl)thiazole)

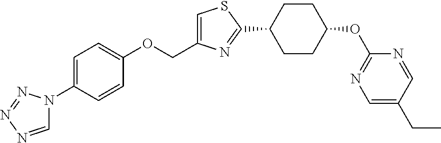

### Trans-(4-((4-(1H-tetrazol-1-yl)phenoxy)methyl)-2-((1r,4r)-4-(5-ethylpyrimidin-2-yloxy)cyclohexyl)thiazole)

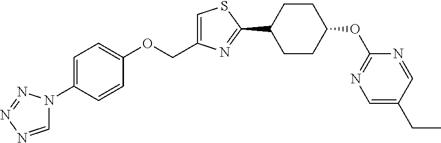

### Step 1. Preparation of 4-(1H-tetrazol-1-yl)phenol

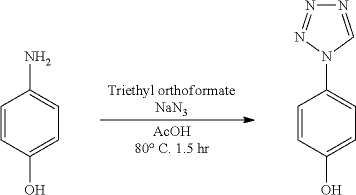

To a stirred solution of 4-aminophenol (5.00 g, 45.8 mmol) in triethyl orthoformate (24.4 mL, 147 mmol) was added sodium azide (3.72 g, 57.3 mmol) and acetic acid (42.0 mL, 733 mmol). The mixture was stirred for 10 min at room temperature and then heated at 80° C. for 1.5 h. Upon heating, the mixture became bronze and homogeneous. The mixture was cooled to room temperature. Water (30.0 mL) and 6 N HCl (17.0 mL) was added. A 25% aqueous solution of NaNO₂ (6.00 mL) was slowly added while the reaction mixture was cooled in an ice bath. The desired product was obtained as a white solid through filtration and washing with water (4.91 g, 66%). ¹H NMR (400 MHz, CD₃OD) δ 6.97 (2H, m), 7.60 (2H, m), 9.57 (1H, s).

### Step 2. Preparation (4-((4-(1H-tetrazol-1-yl)phenoxy)methyl)-2-(4-(5-ethylpyrimidin-2-yloxy)cyclohexyl)thiazole)

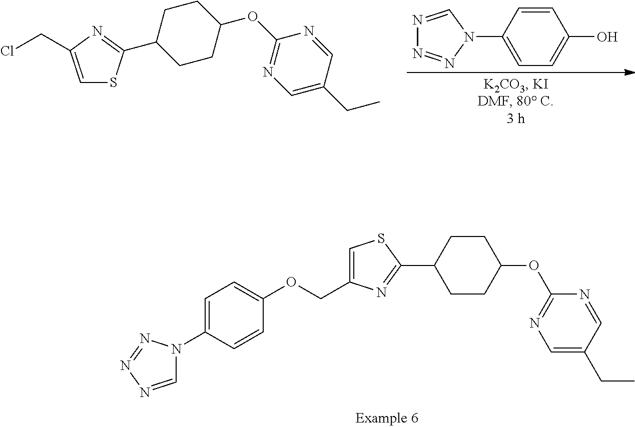

According to the procedure of step 4 in Example 4, the desired compound, Example 6 was obtained. ¹H-NMR (400 MHz, CDCl₃) δ 1.25 (3H, t, J=7.2 Hz), 1.67-1.83 (3H, m), 2.02-2.22 (2H, m), 2.24-2.36 (3H, m), 2.58 (2H, q, J=7.2 Hz), 3.08-3.21 (1H, m), 5.01 and 5.31 (1H, m), 5.24 (2H, s), 7.18 (2H, dd, J=8.8, 1.6 Hz), 7.23 (1H, s), 7.61 (2H, d, J=9.2 Hz), 8.34 (2H, d, J=3.2 Hz), 8.92 (1H, d, J=13.2 Hz). LC-MS Calcd. 463.18 Found 464.0 [M+H]⁺.

### Step 3. Preparation of Example 6—cis & trans

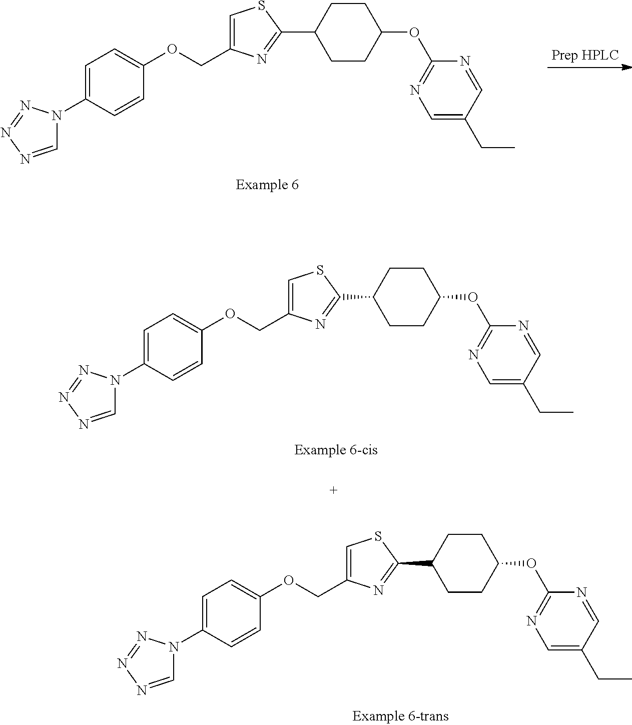

According to the procedure of step 2 in Example 5, the desired product, Example 6—cis (62.0 mg, retention time 9.27 min.) and Example 6-trans (70.0 mg, 8.43 min.) were obtained as a white solid each.

Example 6—cis; (4-((4-(1H-tetrazol-1-yl)phenoxy)methyl)-2-((1s,4s)-4-(5-ethylpyrimidin-2-yloxy)cyclohexyl)thiazole) ¹H-NMR (400 MHz, CDCl₃) δ 1.25 (3H, t, J=7.6 Hz), 1.77-1.83 (2H, m), 2.02-2.25 (6H, m), 2.58 (2H, q, J=7.6 Hz), 3.15-3.20 (1H, m), 5.24 (2H, s), 5.25-5.30 (1H, m), 7.18 (2H, d, J=9.2 Hz), 7.24 (1H, s), 7.62 (2H, d, J=8.8 Hz), 8.35 (2H, s), 8.96 (1H, s). LC-MS Calcd. 463.18 Found 464.2 [M+H]⁺.

Example 6—trans; (4-((4-(1H-tetrazol-1-yl)phenoxy)methyl)-2-((1r,4r)-4-(5-ethylpyrimidin-2-yloxy)cyclohexyl)thiazole). ¹H-NMR (400 MHz, CDCl₃) δ 1.25 (3H, t, J=7.6 Hz), 1.67-1.84 (4H, m), 2.29-2.36 (4H, m), 2.58 (2H, q, J=7.6 Hz), 3.08-3.14 (1H, m), 5.00-5.06 (1H, m), 5.24 (2H, s), 7.18 (2H, d, J=6.8 Hz), 7.24 (1H, s), 7.62 (2H, d, J=6.8 Hz), 8.34 (2H, s), 8.92 (1H, s). LC-MS Calcd. 463.18 Found 464.1 [M+H]⁺.

### Example 7

### Preparation of (N-((2-(4-(5-ethylpyrimidin-2-yloxy)cyclohexyl)thiazol-4-yl)methyl)-4-(methylsulfonyl)aniline)

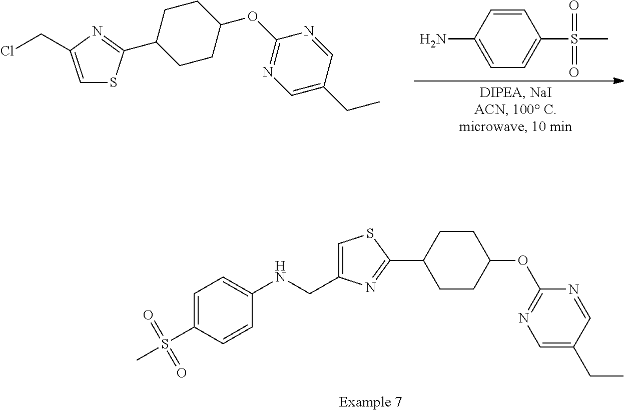

To a solution of 4-(chloromethyl)-2-(4-(5-ethylpyrimidin-2-yloxy)cyclohexyl)thiazole (140 mg, 0.414 mmol) in MeCN (1.80 mL) was added 4-(methylsulfonyl)aniline hydrochloride (170 mg, 0.819 mmol), DIPEA (0.500 mL, 2.87 mmol), and sodium iodide (120 mg, 0.801 mmol) at room temperature. After being heated in a microwave for 10 min at 100° C., the reaction mixture was diluted with water and extracted with EtOAc. The organic layer was washed with brine, dried over anhydrous Na₂SO₄, filtered and concentrated in vacuo. The residue was purified by preparative TLC (Hexnaes:EtOAC=1:1) and then recrystallized from hexanes/EtOAc mixture to give the desired product as a white solid. (34.0 mg, 17.4%) ¹H-NMR (400 MHz, CDCl₃) δ 1.25 (3H, t, J=7.6 Hz), 1.66-1.83 (3H, m), 1.99-2.14 (2H, m), 2.21-2.35 (3H, m), 2.58 (2H, q, J=7.6 Hz), 3.00 (3H, s), 3.07-3.13 (1H, m), 4.46-4.49 (2H, m), 4.95-4.96 (1H, m), 5.00 and 5.31 (1H, m), 6.70 (2H, d, J=8.8 Hz), 7.00 (1H, s), 7.71 (2H, dd, J=8.8, 1.6 Hz), 8.34 (2H, d, J=3.2 Hz). LC-MS Calcd. 472.16 Found 473.1 [M+H]⁺.

### Example 8

### Preparation of 4-((4-(1H-1,2,4-triazol-1-yl)phenoxy)methyl)-2-(4-(5-ethylpyrimidin-2-yloxy)cyclohexyl)thiazole

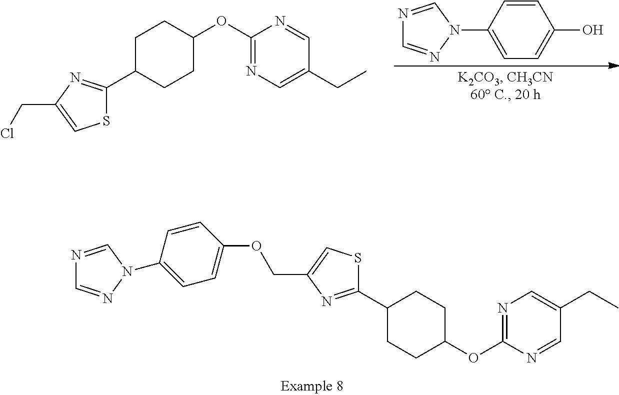

According to the similar procedure of step 4 in Example 4, the desired compound, Example 8 was obtained. NMR (400 MHz, CDCl₃) δ 1.25 (3H, t, J=7.6 Hz), 1.69-1.84 (3H, m), 2.01-2.34 (5H, m), 2.57 (2H, q, J=7.6 Hz), 3.09-3.20 (1H, m), 5.22 (2H, s), 5.00-5.31 (1H, m), 7.09-7.14 (2H, m), 7.22 (1H, s), 7.56-7.61 (2H, m), 8.08 (1H, d, J=1.6 Hz), 8.34 (2H, d, J=3.2 Hz), 8.46 (1H, d, J=2.0 Hz). LC-MS m/z=454.1 [M+H]⁺.

### Example 9

### Preparation of 2-(4-(5-ethylpyrimidin-2-yloxy)cyclohexyl)-4-((2-methyl-4-(1H-tetrazol-1-yl)phenoxy)methyl)thiazole

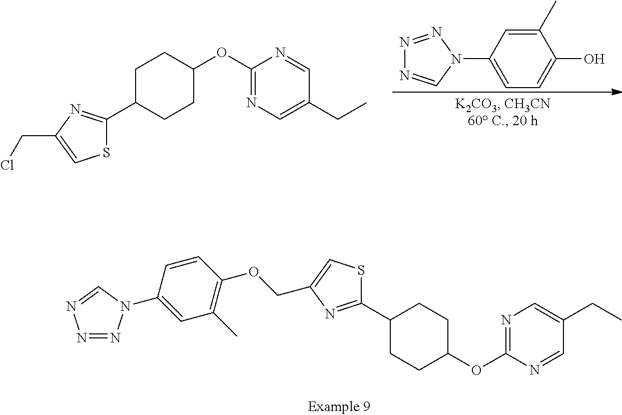

According to the similar procedure of step 4 in Example 4, the desired compound, Example 9 was obtained. ¹H NMR (400 MHz, CDCl₃) δ 1.25 (3H, t, J=7.6 Hz), 1.59-1.84 (3H, m), 2.01-2.38 (8H, m), 2.58 (2H, q, J=7.6 Hz), 3.09-3.17 (1H, m), 5.27 (2H, s), 5.00-5.31 (1H, m), 7.06-7.09 (1H, m), 7.21 (1H, s), 7.44-7.50 (2H, m), 8.34 (2H, d, J=3.2 Hz), 8.90 (1H, d, J=10.8 Hz). LC-MS m/z=477.8 [M+H]⁺.

### Example 10

### Preparation of 2-(4-(5-ethylpyrimidin-2-yloxy)cyclohexyl)-4-((4-(trifluoromethyl)phenoxy)methyl)thiazole

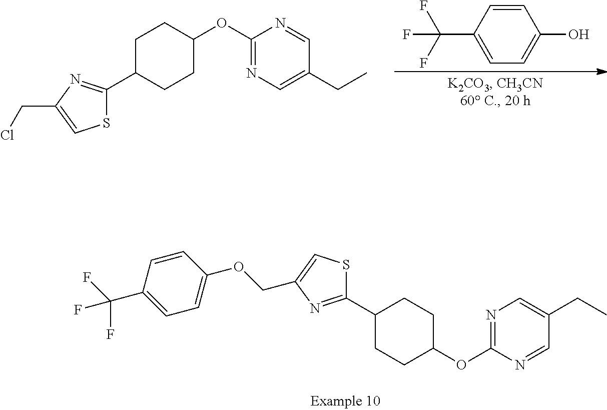

According to the similar procedure of step 4 in Example 4, the desired product was obtained. ¹H NMR (400 MHz, CDCl₃); δ 1.29 (3H, t, J=7.6 Hz), 1.67-1.84 (3H, m), 2.01-2.36 (5H, m), 2.58 (2H, q, J=7.6 Hz), 3.09-3.19 (1H, m), 5.21 (2H, s), 5.00-5.31 (1H, m), 7.06 (2H, d, J=8.8 Hz), 7.20 (1H, s), 7.56 (2H, d, J=8.4 Hz), 8.34 (2H, d, J=3.2 Hz). LC-MS m/z=463.9 [M+H]⁺.

### Example 11

### Preparation of N-(4-((2-(4-(5-ethylpyrimidin-2-yloxy)cyclohexyl)thiazol-4-yl)methoxy)phenyl)methanesulfonamide

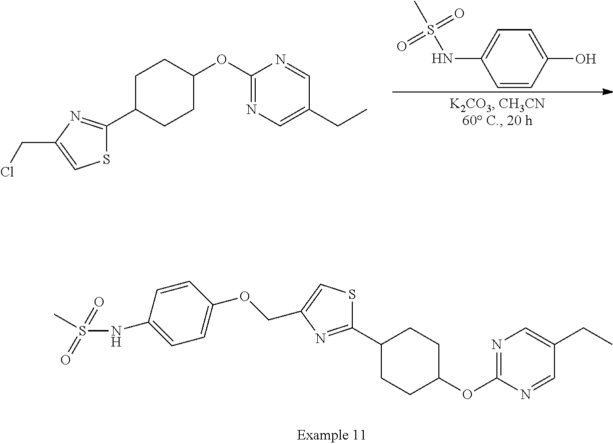

According to the similar procedure of step 4 in Example 4, the desired product was obtained. ¹H NMR (400 MHz, CDCl₃); δ 1.22-1.29 (3H, m), 1.66-2.00 (6H, m), 2.23-2.32 (2H, m), 2.54-2.60 (2H, m), 3.00 (3H, d, J=5.6 Hz), 3.09-3.17 (1H, m), 4.87 (2H, s), 5.00-5.31 (1H, m), 5.16-5.75 (1H, m), 6.72-6.80 (2H, m), 6.95-7.21 (3H, m), 8.34 (2H, d, J=14.0 Hz). LC-MS m/z=489.0 [M+H]⁺.

### Example 12

### Preparation of 2-(4-(5=ethylpyrimidin-2-yloxy)cyclohexyl)-4-((4-(3-methyl-1H-1,2,4-triazol-1-yl)phenoxy)methyl)thiazole

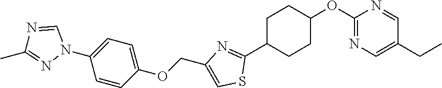

### Step 1. Preparation of 3-methyl-1-(4-nitrophenyl)-1H-1,2,4-triazole

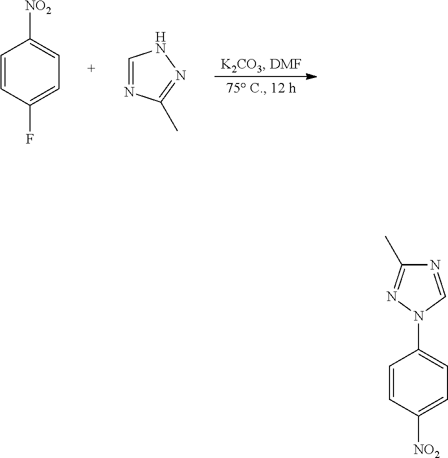

A mixture of 1-fluoro-4-nitrobenzene (1.54 g, 10.9 mmol), 3-methyl-1H-1,2,4-triazole (1.00 g, 12.0 mmol) and K₂CO₃ (1.66 g, 12.0 mmol) in DMF (30 ml) was stirred at 75° C. for 12 hours. After being cooled to room temperature, the reaction mixture was concentrated in vacuo. The mixture was dissolved in EtOAc, washed with saturated aq. NaHCO₃, dried over Na₂SO₄, filtered and concentrated in vacuo. The residue was purified by column chromatography on SiO₂ (Hex:EA=2:1) to give the desired product (0.500 g, 22%) as a brown solid. ¹H NMR (400 MHz, CDCl₃) δ 2.52 (3H, s), 7.85-7.88 (2H, m), 8.37-8.40 (2H, m), 8.58 (1H, s). LC-MS m/z=205.0 [M+H]⁺.

### Step 2. Preparation of 4-(3-methyl-1H-1,2,4-triazol-1-yl)aniline

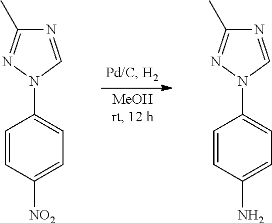

A mixture of 3-methyl-1-(4-nitrophenyl)-1H-1,2,4-triazole (480 mg, 2.35 mmol) and Pd/C (75.0 mg, 0.071 mmol) in MeOH (25.0 mL) was stirred for 12 hours at room temperature under H₂ atmosphere (balloon). The reaction mixture was filtered through a celite-pad and concentrated in vacuo to afford the desired product (410 mg, 100%) which was used without further purification. LC-MS m/z=175.0 [M+H]⁺.

### Step 3. Preparation of 4-(3-methyl-1H-1,2,4-triazol-1-yl)phenol

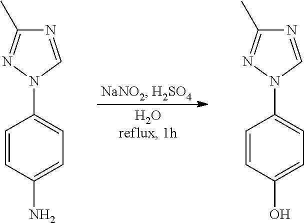

To a suspension of 4-(3-methyl-1H-1,2,4-triazol-1-yl)aniline (130 mg, 0.744 mmol) in H₂O (4.00 mL) and conc. sulfuric acid (0.400 mL) was added a solution of NaNO₂ (77.0 mg, 1.12 mmol) in H₂O (4.00 mL) at 0° C. The mixture was stirred at 0° C. for 30 minutes and then additional water (2.00 mL) and sulfuric acid (0.740 mL) was added. The reaction mixture was heated at reflux for 1 hour, cooled and extracted with EtOAc. The combined organic layers were washed with brine, dried over Na₂SO₄, filtered, concentrated in vacuo and purified by column chromatography on SiO₂ (Hex:EA=1:1) to give the desired product (75.0 mg, 50%) as a white solid. ¹H NMR (400 MHz, CDCl₃) δ 2.49 (3H, s), 6.91-6.96 (2H, m), 7.46-7.50 (2H, m), 8.32 (1H, s). LC-MS m/z=176.0 [M+H]⁺.

### Step 4. Synthesis of 2-(4-(5-ethylpyrimidin-2-yloxy)cyclohexyl)-4-((4-(3-methyl-1H-1,2,4-triazol-1-yl)phenoxy)methyl)thiazole

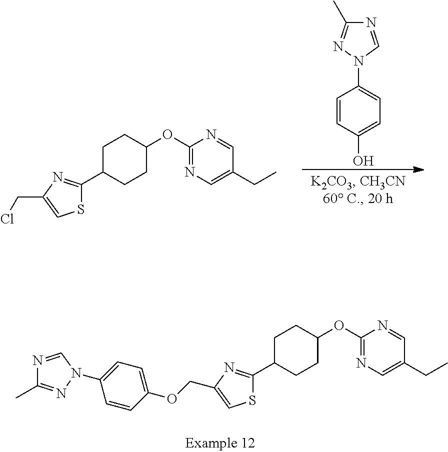

To a mixture of 4-(3-methyl-1H-1,2,4-triazol-1-yl)phenol (31.1 mg, 0.178 mmol) and K₂CO₃ (61.4 mg, 0.444 mmol) in CH₃CN (2.00 mL) was added 4-(chloromethyl)-2-(4-(5-ethylpyrimidin-2-yloxy)cyclohexyl)thiazole (50.0 mg, 0.148 mmol) at room temperature. The reaction mixture was stirred at 60° C. for 20 hours, cooled to room temperature and partitioned between EtOAc and water. The organic layer was dried over MgSO₄, filtered and concentrated in vacuo. The residue was purified by prep-TLC to give the desired product (40.0 mg, 57%) as a white solid. ¹H NMR (400 MHz, CDCl₃) δ 1.22-1.29 (3H, m), 1.66-1.84 (3H, m), 2.01-2.35 (5H, m), 2.49 (3H, s), 2.58 (2H, q, J=7.6 Hz), 3.07-3.20 (1H, m), 5.00-5.30 (1H, m), 5.21 (2H, s), 7.09 (2H, d, J=8.8 Hz), 7.22 (1H, s), 7.54 (2H, dd, J=8.8, 2.0 Hz), 8.32-8.35 (3H, m). LC-MS m/z=476.9 [M+H]⁺.

### Example 13

### Preparation of 2-(4-(5-ethylpyrimidin-2-yloxy)cyclohexyl)-4-((4-(5-methyl-2H-tetrazol-2-yl)phenoxy)methyl)thiazole

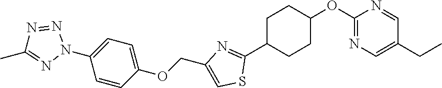

### Step 1. Preparation of 5-methyl-2-(4-nitrophenyl)-2H-tetrazole

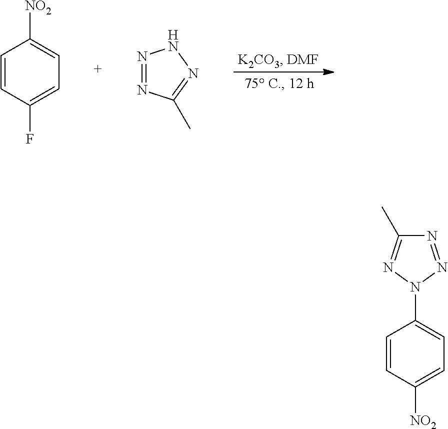

According to the similar procedure of step 1 in Example 12, the desired product was obtained. ¹H NMR (400 MHz, CDCl₃) δ 2.69 (3H, s), 8.31-8.36 (2H, m), 8.42-8.46 (2H, m). LC-MS m/z=205.9 [M+H]⁺.

### Step 2. Preparation of 4-(5-methyl-2H-tetrazol-2-yl)aniline

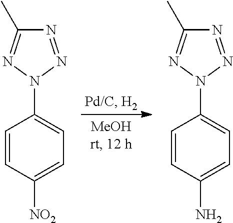

According to the similar procedure of step 2 in Example 12, the desired product was obtained. LC-MS m/z=175.8 [M+H]⁺.

### Step 3. Preparation of 4-(5-methyl-2H-tetrazol-2-yl)phenol

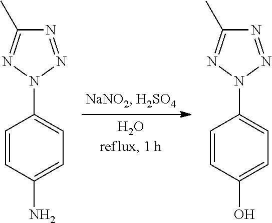

According to the similar procedure of step 3 in Example 12, the desired product was obtained. ¹H NMR (400 MHz, CDCl₃) δ 2.63 (3H, s), 6.97-7.01 (2H, m), 7.94-7.98 (2H, m). LC-MS m/z=177.0 [M+H]⁺.

### Step 4. Preparation of 2-(4-(5-ethylpyrimidin-2-yloxy)cyclohexyl)-4-((4-(5-methyl-2H-tetrazol-2-yl)phenoxy)methyl)thiazole

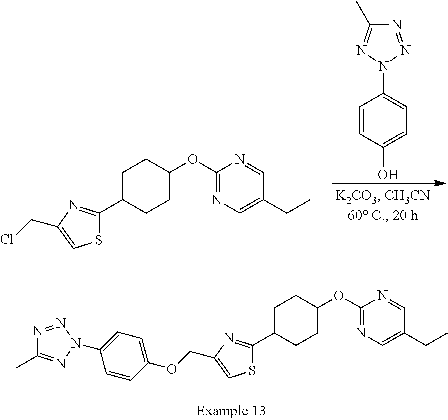

According to the similar procedure of step 4 in Example 12, the desired product was obtained. ¹H NMR (400 MHz, CDCl₃) δ 1.25 (3H, t, J=7.6 Hz), 1.60-1.84 (3H, m), 2.03-2.36 (5H, m), 2.58 (2H, q, J=7.6 Hz), 2.62 (3H, s), 3.10-3.17 (1H, m), 5.00-5.30 (1H, m), 5.23 (2H, s), 7.13 (2H, d, J=9.2 Hz), 7.23 (1H, s), 8.01 (2H, dd, J=9.2, 2.0 Hz), 8.34 (2H, d, J=3.2 Hz). LC-MS m/z=477.9 [M+H]⁺.

### Example 14

### Preparation of benzyl 4-(4-((4-(1H-tetrazol-1-yl)phenoxy)methyl)thiazol-2-yl)cyclohexyl carbamate

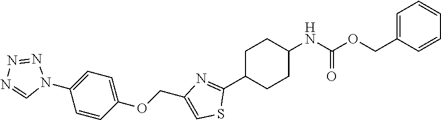

### Step 1. Preparation of benzyl 4-cyanocyclohexylcarbamate

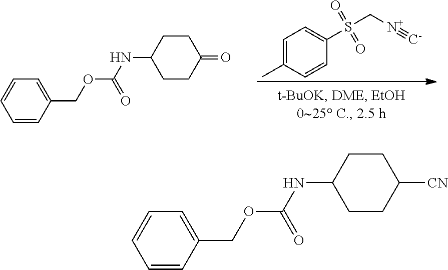

To a solution of benzyl 4-oxocyclohexylcarbamate (2.00 g, 8.09 mmol) and 1-(isocyanomethylsulfonyl)-4-methylbenzene (1.82 g, 9.30 mmol) in DME (19.4 mL)/EtOH (0.78 mL) was added t-BuOK (1.82 g, 16.18 mmol) portionwise at 0° C. After being stirred for 2.5 h at room temperature, the precipitate was removed by filtration. The filtrate was concentrated in vacuo and the residue was purified by column chromatography on SiO₂ (Hex:EtOAc=2:1) to give the desired product (1.6 g, 77%) as a white solid. ¹H NMR (400 Hz, CDCl₃) δ 1.15-1.26 (2H, m), 1.62-1.74 (2H, m), 1.98-2.13 (4H, m), 2.40 (1H, m), 3.55 (1H, brs), 4.71 (1H, d, J=6.0 Hz), 5.10 (2H, s), 7.32-7.39 (5H, m). LC-MS m/z=258.8 [M+H]⁺.

### Step 2. Preparation of benzyl 4-carbamothioylcyclohexylcarbamate

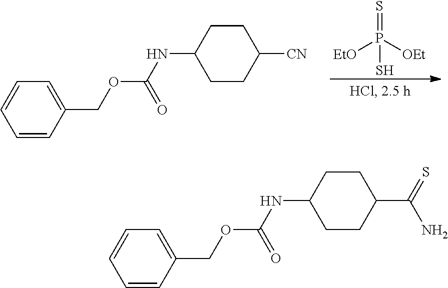

To a solution of benzyl 4-cyanocyclohexylcarbamate (1.50 g, 5.81 mmol) in HCl (4.0 M in dioxane, 10.2 mL, 40.6 mmol) was added O,O-diethyl S-hydrogen phosphorodithioate (0.89 mL, 5.81 mmol) at 0° C. The reaction mixture was stirred for 2.5 hour at room temperature. The mixture was concentrated in vacuo. The residue was quenched with saturated aq. NaHCO₃ and extracted with EtOAc. The organic layer was washed with water and brine, dried over Na₂SO₄, filtered and concentrated in vacuo. The formed solid was filtered and rinsed with Et₂O to give the desired product (1.40 g, 82%) as a white solid. ¹H NMR (400 Hz, DMSO) δ 1.14-1.23 (2H, m), 1.52-1.61 (2H, m), 1.68-1.70 (2H, m), 1.83-1.85 (2H, m), 2.38-2.45 (1H, m), 4.99 (2H, s), 7.18 (1H, d, J=8.0 Hz), 7.39-7.29 (5H, m), 9.04 and 9.32 (2H, each s). LC-MS m/z=292.9 [M+H]⁺.

### Step 3. Preparation of benzyl 4-(4-(chloromethyl)thiazol-2-yl)cyclohexylcarbamate

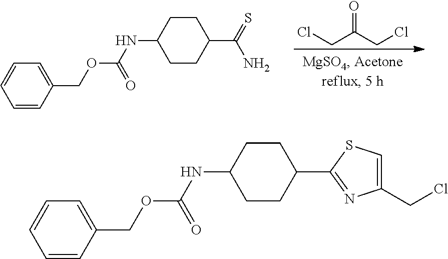

To a solution of benzyl 4-carbamothioylcyclohexylcarbamate (1.40 g, 4.79 mmol) in acetone (23.9 mL) was added 1,3-dichloropropan-2-one (0.79 g, 6.22 mmol) and MgSO₄ (0.75 g, 6.22 mmol) at rt. After being stirred under reflux for 5 hours, the reaction mixture was diluted with water and extracted with EtOAc. The organic layer was washed with brine, dried over Na₂SO₄, filtered and concentrated in vacuo. The residue was purified by column chromatography on SiO₂ (Hex:EtOAc=4:1) to give the desired product (1.10 g, 63%) as a colorless oil. ¹H NMR (400 Hz, CDCl₃) δ 1.23-1.33 (2H, m), 1.60-1.70 (2H, m), 2.16-2.30 (4H, m), 2.93-3.00 (1H, m), 3.59 (1H, brs), 4.86 (2H, s), 5.10 (2H, s), 7.17 (1H, s), 7.31-7.39 (5H, m). LC-MS m/z=364.9 [M+H]⁺.

### Step 4. Preparation of benzyl 4-(4-((4-(1H-tetrazol-1-yl)phenoxy)methyl)thiazol-2-yl)cyclohexylcarbamate

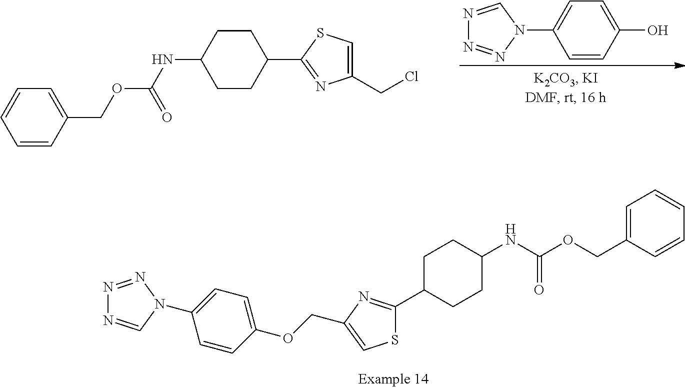

A mixture of benzyl 4-(4-(chloromethyl)thiazol-2-yl)cyclohexylcarbamate (1.10 g, 3.01 mmol), 4-(1H-tetrazol-1-yl)phenol (0.538 g, 3.32 mmol), K₂CO₃ (0.83 g, 6.03 mmol) and K1 (0.100 g, 0.603 in DMF (15.0 mL) was stirred at 80° C. for 3 hours. The reaction mixture was diluted with water and extracted with EtOAc. The separated organic layer was washed with brine, dried over anhydrous Na₂SO₄, filtered and concentrated in vacuo. The residue was purified by column chromatography on SiO₂ (Hex.:EtOAc=2:1 to 1:3) to give the desired product (510 mg, 34%) as a pale yellow solid. NMR (400 Hz, CDCl₃) δ 1.23-1.35 (2H, m), 1.78-1.90 (4H, m), 2.18-2.26 (2H, m), 3.10-3.15 (1H, m), 4.65 and 4.91 (1H, each s), 5.10 (2H, s), 5.22 (2H, s), 7.17 (2H, d, J=8.8 Hz), 7.23 (1H, d, J=3.6 Hz), 7.31-7.37 (5H, m), 7.61 (2H, d, J=9.2 Hz), 8.02 (1H, brs), 8.90 (1H, s). LC-MS m/z=491.0 [M+H]⁺.

### Example 15

### Step 1. Preparation of 1-(2-(4-(5-ethylpyrimidin-2-yloxy)cyclohexyl)thiazol-4-yl)-N-(4-(methylsulfonyl)benzyl)methanamine

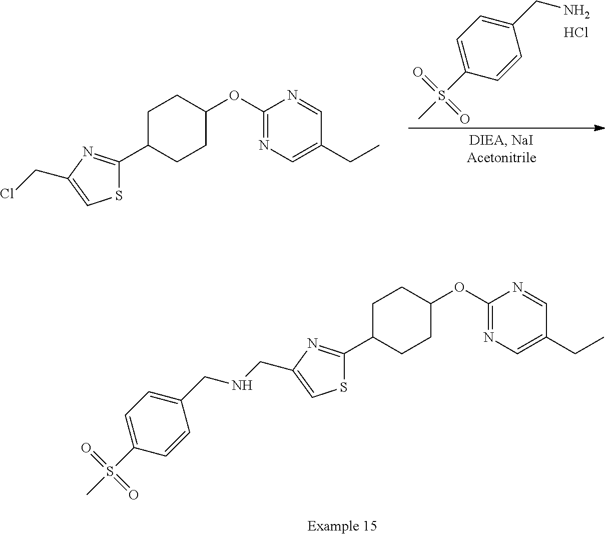

According to the similar procedure of Example 7, the desired product was obtained. ¹H NMR (400 MHz, CDCl₃) δ 1.22-1.29 (3H, m), 1.66-1.84 (3H, m), 2.01-2.35 (5H, m), 2.58 (2H, q, J=7.6 Hz), 3.07-3.20 (4H, m), 3.86-3.95 (4H, m), 5.00-5.30 (1H, m), 6.97 (1H, s), 7.58 (2H, d, J=8.4 Hz), 7.90 (2H, d, J=7.6 Hz), 8.34 (2H, d, J=4.0 Hz). LC-MS m/z=486.8 [M-FH]⁺.

### Example 16

### Preparation of 2-(4-(5-ethylpyrimidin-2-yloxy)cyclohexyl)-4-((6-(methylsulfonyl)-5,6,7,8-tetrahydropyrido[4,3-d]pyrimidin-2-yloxy)methyl)thiazole

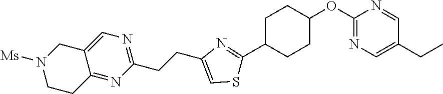

### Step 1. Preparation of (Z)-1-benzyl-3-((dimethylamino)methylene)piperidin-4-one

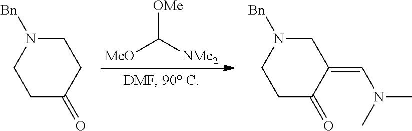

To a stirred solution of 1-benzylpiperidin-4-one (4.0 g, 21.1 mmol) in DMF (4 mL) was added 1,1-dimethoxy-N,N-dimethylmethanamine (2.8 g, 23.3 mmol) at room temperature. The mixture was stirred for 18 hours at 90° C. The mixture was cooled to room temperature and the mixture purified by column chromatography on SiO₂ (Hex.:EtOAc=1:1 to DCM:MeOH=15:1) to give the desired product (4.2 g, 81%) as a brown oil. ¹H NMR (400 MHz, CDCl₃) δ 2.45 (2H, d, J=6.4 Hz), 2.69 (2H, d, J=6.0 Hz), 3.01 (6H, s). 3.60 (2H, s), 3.64 (2H, s), 7.33-7.38 (5H, m), 7.46 (1H, s). LC-MS m/z=245.0 [M+H]⁺.

### Step 2. Preparation of 6-benzyl-2-methoxy-5,6,7,8-tetrahydropyrido[4,3-d]pyrimidine

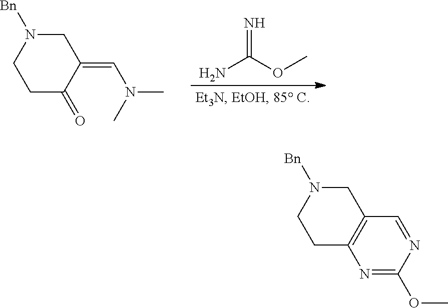

A stirred mixture of (Z)-1-benzyl-3-((dimethylamino)methylene)piperidin-4-one (3.5 g, 14.32 mmol), O-methylisourea hydrogensulfate (1.6 g, 21.5 mmol) and triethyl amine (4 mL, 28.64 mmol) in ethanol 60 mL was heated for 18 hours at 85° C. Ethanol was removed in vaccuo and the residue was extracted with H₂O/EtOAc (50 mL×1/100 mL×3). The combined organic layers were dried over anhydrous MgSO₄, filtered and concentrated in vacuo. The residue was purified by column chromatography on SiO₂ (Hex:EtOAc=1:1 to EtOAc) to give the desired product (0.6 g, 16.4%) as a yellow oil. ¹H NMR (400 MHz, CDCl₃) δ 2.81 (2H, d, J=6.0 Hz), 2.92 (2H, d, J=6.0 Hz), 3.53 (2H, s), 3.71 (2H, s), 3.97 (3H, s), 7.28-7.36 (5H, m), 8.12 (1H, s). LC-MS m/z=256.1 [M+H]⁺.

### Step 3. Preparation of 2-methoxy-6-(methylsulfonyl)-5,6,7,8-tetrahydropyrido[4,3-d]pyrimidine

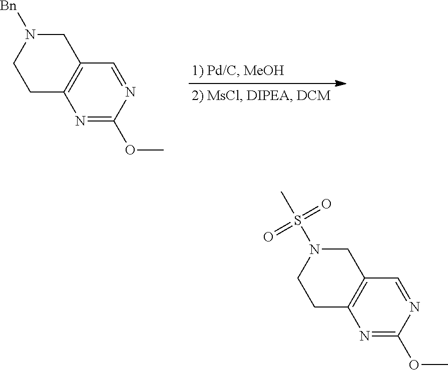

To a solution of 6-benzyl-2-methoxy-5,6,7,8-tetrahydropyrido[4,3-d]pyrimidine (600 mg, 2.35 mmol) in methanol 20 mL was added Pd/C (5% on activated carbon, 300 mg) in small portions. The mixture was hydrogenated overnight at room temperature under a hydrogen atmosphere (balloon). Methanol was completely removed in vaccuo and the residue was suspended in anhydrous DCM (20 mL) and treated with DIPEA (0.8 mL, 4.7 mmol). MsCl (0.27 mL, 3.53 mmol) in DCM (1 mL) was added to this mixture dropwise while stirring at 0° C. The mixture was stirred for 1 hour at room temperature, concentrated and the residue was purified by column chromatography on SiO₂ (EtOAc as an eluant) to give the desired product (200 mg, 35%) as a white solid. ¹H NMR (400 MHz, CDCl₃) δ 2.90 (3H s,), 3.03 (2H, t, J=6.4 Hz), 3.64 (2H, t, J=6.0 Hz), 4.00 (3H, s), 4.42 (2H, s), 8.23 (1H, s). LC-MS m/z=244.0 [M+H]⁺.

### Step 4. Preparation of 6-(methylsulfonyl)-5,6,7,8-tetrahydropyrido[4,3-d]pyrimidin-2-ol

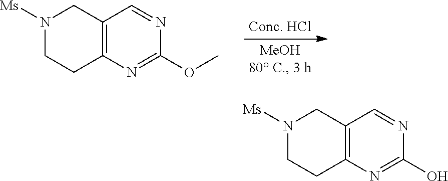

To a stirred solution of 2-methoxy-6-(methylsulfonyl)-5,6,7,8-tetrahydropyrido[4,3-d]pyrimidine (260 mg, 1.07 mmol) in MeOH (0.33 mL) was added conc. HCl (1.00 mL, 12.0 mmol). The reaction mixture was heated to 80° C. for 3 h and then cooled to room temperature. The mixture was concentrated to give the desired compound (240 mg, 99%) as a white solid. ¹H NMR (400 MHz, CDCl₃) δ 2.55 (2H, t, J=6.0 Hz), 2.91 (3H, s), 3.36 (2H, t, J=6.0 Hz), 4.05 (2H, s), 7.83 (1H, s). LC-MS m/z=230.0 [M+H]⁺.

### Step 5. Preparation of 2-(4-(5-ethylpyrimidin-2-yloxy)cyclohexyl)-4-((6-(methylsulfonyl)-5,6,7,8-tetrahydropyrido[4,3-d]pyrimidin-2-yloxy)methyl)thiazole

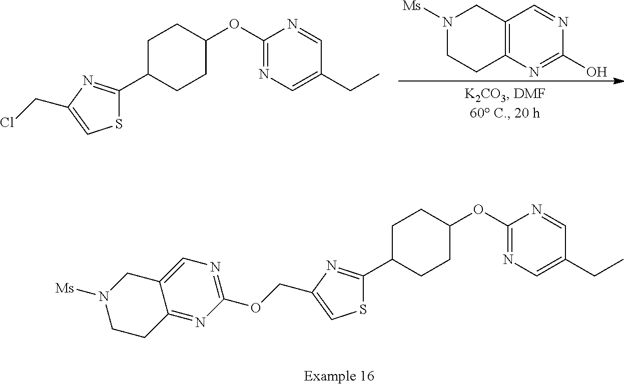

According to the similar procedure of step 4 in Example 4, the desired product was obtained. ¹H NMR (400 MHz, CDCl₃) δ 1.22-1.29 (3H, m), 1.66-1.85 (3H, m), 2.01-2.35 (5H, m), 2.50-2.65 (2H, m), 2.90 (3H, s), 2.95-3.00 (2H, m), 3.07-3.20 (1H, m), 3.55-3.65 (2H, m), 4.30 (2H, s), 5.00-5.30 (1H, m), 5.21 (2H, s), 7.37 (1H, s), 7.85 (1H, d, J=18.4 Hz), 8.34 (2H, s). LC-MS m/z=530.9 [M+H]⁺.

### Example 17

### Preparation of Preparation of 2-(4-(benzyloxy)cyclohexyloxy)-5-bromo-1,3-difluorobenzene

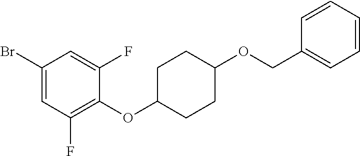

### Step 1. Preparation of 8-(benzyloxy)-1,4-dioxaspiro[4.5]decane

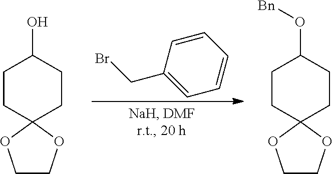

A mixture of 1,4-dioxaspiro[4.5]decan-8-ol (6.656 g, 42.1 mmol) and NaH (1.616 g, 67.3 mmol) in THR (20.0 mL) was stirred at room temperature for 30 minutes, followed by the addition of benzyl bromide (5.91 ml, 49.6 mmol). The reaction was stirred at room temperature for 15 hours. The reaction was quenched with saturated ammonium chloride and extracted with EtOAc. The organic phase was washed with brine, dried over magnesium sulfate, filtered and concentrated. The residue was purified by column chromatography on SiO₂ to give the desired product (6.57 g, 63%). ¹H-NMR (400 MHz. CDCl₃) δ 1.54 (2H, m), 1.83 (6H, m), 3.51 (1H, m), 3.92 (4H, m), 4.52 (2H, s), 7.25-7.32 (5H, m).

### Step 2. Preparation of 4-(benzyloxy)cyclohexanol

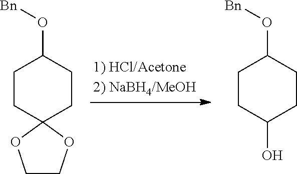

According to the similar procedure of steps 2 & 3 in Example 3, the desired product was obtained. ¹H-NMR (400 MHz, CDCl₃) δ 1.65 (8H, m), 3.40-3.49 (1H, m), 3.67 (1H, m), 4.52 (2H, d, J=8.8 Hz), 7.24-7.31 (5H, m).

### Step 3. Preparation of 4-(benzyloxy)cyclohexyl methanesulfonate

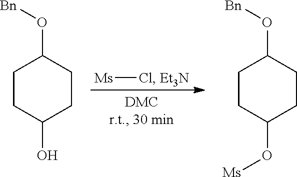

4-(Benzyloxy)cyclohexanol (1.9656 g, 9.53 mmol) was dissolved in DCM (10.0 treated with Et₃N (2.66 ml, 19.06 mmol) and cooled to 0° C. Methanesulfonylchloride (0.824 ml, 10.58 mmol) was added dropwise and the mixture was stirred at room temperature for 2 hours. The reaction mixture was quenched by addition of water and extracted with EtOAc. The organic layer was washed with brine and dried over anhydrous MgSO₄, filtered and concentrated in vacuo. The residue was purified by column chromatography on SiO₂ to give the desired product (2.6679 g, 9.38 mmol, 98% yield). ¹H-NMR (400 MHz, CDCl₃) δ 1.78 (8H, m), 3.01 (3H, s), 3.51 (1H, m), 4.52 (2H, s), 4.76 (1H, m), 7.26-7.33 (5H, m).

### Step 4. Preparation of 2-(4-(benzyloxy)cyclohexyloxy)-5-bromo-1,3-difluorobenzene

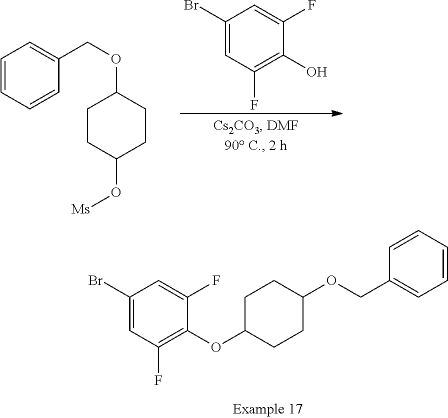

To a stirred solution of 4-(benzyloxy)cyclohexyl methanesulfonate (2.6679 g, 9.38 mmol) and 4-bromo-2,6-difluorophenol (2.157 g, 10.32 mmol) in DMF (10 ml) was added Cs₂CO₃ (4.71 g, 14.45 mmol). The resulting suspension was heated to 90° C. for 2 hours, cooled to room temperature, and diluted with H₂O (100 ml). The mixture was extracted with EtOAc, and the combined organic layers were washed with brine, dried over MgSO₄, and concentrated in vacuo. Purification by flash column chromatography on SiO₂ afforded the desired product (3.7 g, 99%). ¹H-NMR (400 MHz, CDCl₃) δ 1.94 (8H, m), 3.45-3.52 (1H, s), 4.52 (2H, m), 5.60 (1H, m), 7.05 (2H, m), 7.24-7.32 (5H, m).

### Example 18

### Preparation of 4-(4-(benzyloxy)cyclohexyloxy)-3,5-difluoro-4′-(methylsulfonyl)biphenyl

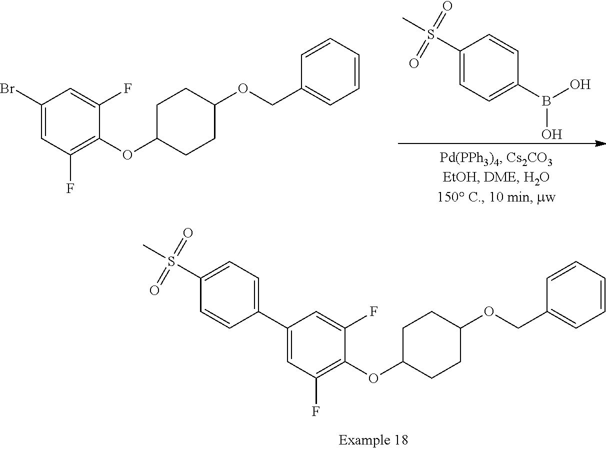

A mixture of 2-(4-(benzyloxy)cyclohexyloxy)-5-bromo-1,3-difluorobenzene (80 mg, 0.201 mmol), 4-(methylsulfonyl)phenylboronic acid (48.3 mg, 0.242 mmol), Cs₂CO₃ (197 mg, 0.604 mmol) and Pd(Ph₃P)₄ (6.98 mg, 6.04 μmol in H₂O (0.24 ml), EtOH (0.36 ml) and DME (0.72 ml) was heated via microwave irradiation (150° C., 10 min). After being cooled to room temperature, the reaction mixture was extracted with EtOAc and water. The organic phase was separated, dried (MgSO₄), filtered, concentrated in vacuo and purified by flash chromatography to afford the desired product (61.2 mg, 0.130 mmol, 64.3% yield). ¹H-NMR (400 MHz, CDCl₃) δ 1.63 (4H, m), 2.01 (4H, m), 3.09 (3H, s), 3.51 (1H, m), 4.35 (1H, m), 4.52 (2H, d, J=20.0 Hz), 7.15 (2H, m), 7.34 (5H, m), 7.95 (4H, m).

### Example 19

### Preparation of 4-(3,5-difluoro-4′-(methylsulfonyl)biphenyl-4-yloxy)cyclohexanol

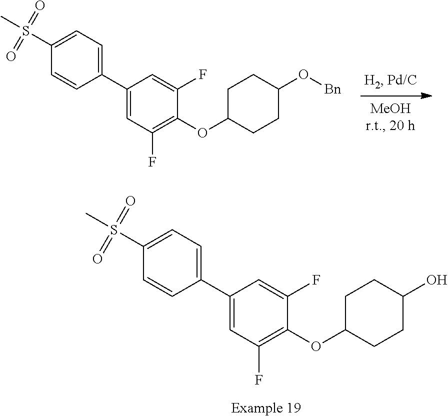

A mixture of 4-(4-(benzyloxy)cyclohexyloxy)-3,5-difluoro-4′-(methylsulfonyl)biphenyl (61.2 mg, 0.130 mmol) and Pd/C (13.78 mg, 10%, 0.013 mmol) in MeOH (10 mL) was stirred at room temperature for 20 hours under a hydrogen atmosphere (balloon). After filtration through a Celite pad, the filtrate was concentrated in vacuo. The residue was purified by flash chromatography to give the desired product (34.4 mg, 70%) as a white solid. ¹H-NMR (400 MHz. CDCl₃) δ 1.74-1.87 (5H, m), 2.08 (3H, m), 3.09 (3H, s), 3.83 (1H, m), 4.26-4.40 (1H, m), 7.15 (2H, m), 7.69 (2H, m), 8.01 (2H, m).

### Example 20

### Preparation of 2-(4-(4-bromo-2,6-difluorophenoxy)cyclohexyloxy)-5-ethylpyrimidine

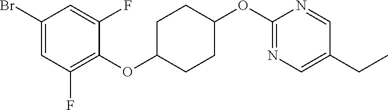

### Step 1. Preparation of 4-(5-ethylpyrimidin-2-yloxy)cyclohexyl methanesulfonate

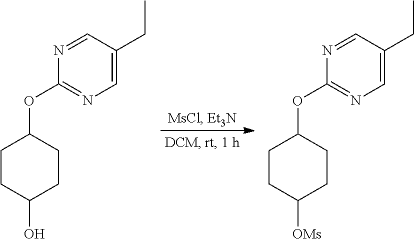

To a stirred solution of 4-(5-ethylpyrimidin-2-yloxy)cyclohexanol (100 mg, 0.450 mmol) and Et₃N (0.125 mL, 0.900 mmol) in DCM (2 mL) was added methanesulfonylchloride (0.039 mL, 0.499 mmol) dropwise at 0° C. The mixture was stirred for 1 hour at room temperature and was quenched by addition of water. The mixture was extracted with EtOAc. The organic layer was washed with brine, dried over anhydrous MgSO₄, filtered and concentrated in vacuo to give the desired product which was used for the next step without further purification. LC-MS Calcd. 300.11, Found 300.82 [M+H]⁺.

### Step 2. Preparation of 2-(4-(4-bromo-2,6-difluoro phenoxy)cyclohexyl oxy)-5-ethyl pyrimidine

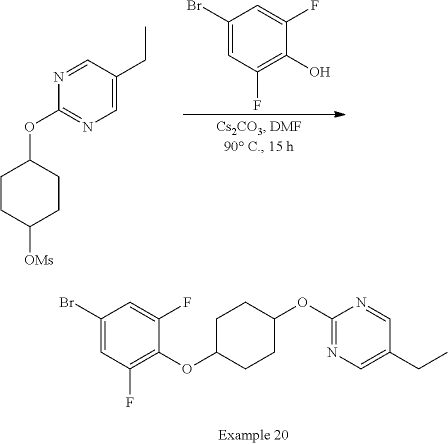

To a stirred solution of 4-(5-ethylpyrimidin-2-yloxy)cyclohexyl methanesulfonate (135 mg, 0.449 mmol) and 4-Bromo-2,6-difluorophenol (103 mg, 0.494 mmol) in DMF (3 mL) was added CS₂CO₃ (226 mg, 0.692 mmol) at room temperature. The resulting suspension is heated to 90° C. for 15 h. After being cooled to room temperature, the mixture was diluted with water, extracted with EtOAc. The combined organic layer was washed with brine, dried over MgSO₄, and concentrated in vacuo. Purification by flash chromatography on SiO₂ provided desired product (59 mg, 32%). LC-MS Calcd. 412.06, Found 412.90 [M+H]⁺.

### Example 21

### Preparation of 4-(3,5-difluoro-4′-(methylsulfonyl)biphenyl-4-yloxy)cyclohexyl pentanoate

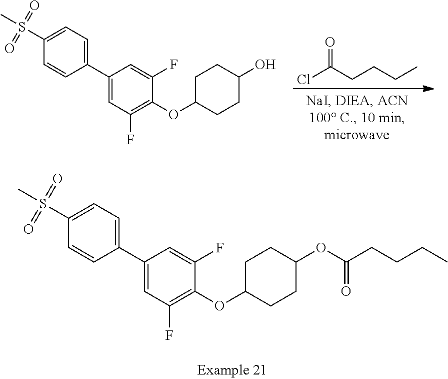

To a stirred solution of 4-(3,5-difluoro-4′-(methylsulfonyl)biphenyl-4-yloxy)cyclohexanol (40 mg, 0.105 mmol) in ACN (0.5 ml) was added DIEA (0.073 ml, 0.418 mmol), sodium iodide (31.4 mg, 0.209 mmol) and valeroyl chloride (25 μL, 0.210 mmol). The mixture was heated at 100° C. for 10 minutes in a microwave. After being cooled, the reaction mixture was extracted with EtOAc and water. The organic phase was separated, dried (MgSO₄), filtered and purified by flash chromatography to afford the desired product (49 mg. 80%). ¹H-NMR (400 MHz, CDCl₃) δ 0.90 (3H, m), 1.30-2.09 (17H, m), 2.31 (2H, m), 3.21 (3H, s), 4.35 (1H, m), 4.91 (1H, m), 7.16 (2H, m), 7.70 (2H, d, J=8.8 Hz), 8.01 (2H, d, J=6.8 Hz).

### Example 22

### Preparation of 4-(3,5-difluoro-4′-(methylsulfonyl)biphenyl-4-yloxy)cyclohexyl 3-methyl butanoate

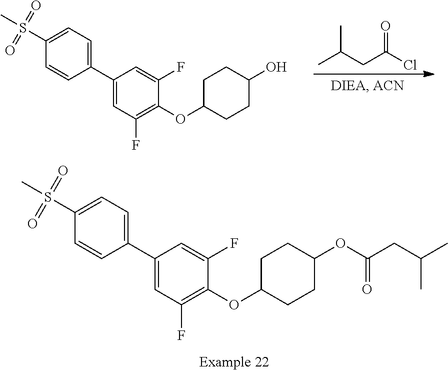

According to the similar procedure of Example 21, the desired product was obtained. ¹H-NMR (400 MHz, CDCl₃) δ 0.95 (6H, m), 1.54 (1H, m), 1.76 (2H, m), 2.08 (8H, m), 3.10 (3H, s), 4.34 (1H, m), 4.91 (1H, m), 7.17 (2H, m), 7.70 (2H, d, J=8.4 Hz), 8.00 (2H, d, J=10.8 Hz).

### Example 23

### Preparation of 4-(3,5-difluoro-4′-(methylsulfonyl)biphenyl-4-yloxy)cyclohexanamine

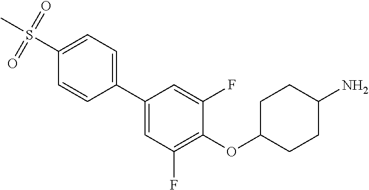

### Step 1. Preparation of 4-(3,5-difluoro-4′-(methylsulfonyl)biphenyl-4-yloxy)cyclohexyl methanesulfonate

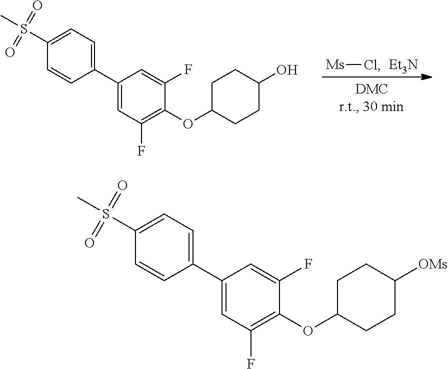

4-(3,5-Difluoro-4′-(methylsulfonyl)biphenyl-4-yloxy)cyclohexanol (230.2 mg, 0.602 mmol) was dissolved in DCM (2 ml), treated with Et₃N (0.168 ml, 1.204 mmol) and cooled to 0° C. Methanesulfonyl chloride (0.052 ml, 0.668 mmol) was added dropwise to this stirred mixture. The ice bath was removed and the stirring continued for 30 minutes. The reaction mixture was quenched by addition of water and then extracted with EtOAc. The organic layer was washed with brine, dried over anhydrous MgSO₄, filtered and concentrated in vacuo. The residue was purified by flash chromatography on SiO₂ to afford the desired product (82.3 mg, 30%). ¹H-NMR (400 MHz, CDCl₃) δ 1.83 (4H, m), 2.07 (2H, m), 2.23 (2H, m). 3.03 (3H, s), 3.09 (3H, s), 4.42 (1H, m), 4.92 (1H, m), 7.18 (2H, m), 7.70 (2H, m), 8.01 (2H, m).

### Step 2. Preparation of 4-(4-azidocyclohexyloxy)-3,5-difluoro-4′-(methylsulfonyl)biphenyl

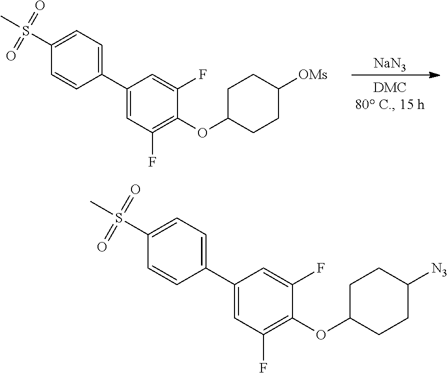

To a stirred solution of 4-(3,5-difluoro-4′-(methylsulfonyl)biphenyl-4-yloxy)cyclohexyl methanesulfonate (82.3 mg, 0.179 mmol) in DMF (2 ml) was added sodium azide (34.9 mg, 0.536 mmol) and the resulting suspension stirred at 90° C. for 15 hours. The reaction mixture was quenched by addition of water and then extracted with EtOAc. The organic layer was washed with brine and dried over anhydrous MgSO₄, filtered and concentrated in vacuo. The residue was purified by column chromatography on SiO₂ to afford the desired product (68 mg, 93%). ¹H-NMR (400 MHz, CDCl₃) δ 1.76 (4H, m), 2.03 (4H, m), 3.09 (3H, s), 3.51 (1H, m), 4.40 (1H, m), 7.17 (2H, m), 7.69 (2H, m), 8.01 (2H, m).

### Step 3. Preparation of 4-(3,5-difluoro-4′-(methylsulfonyl) biphenyl-4-yloxy)cyclohexanamine

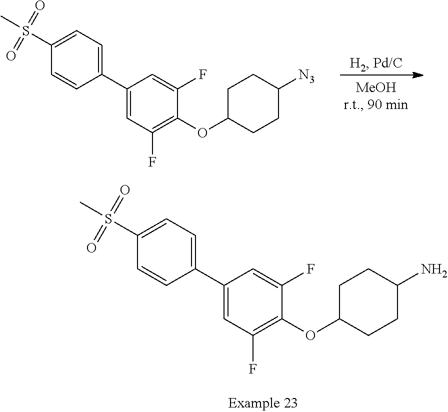

A mixture of 4-(4-azidocyclohexyloxy)-3,5-difluoro-4′-(methylsulfonyl)biphenyl (21.5 mg, 0.053 mmol) and Pd/C (5.62 mg, 5.28 μmol in MeOH (5 ml) was stirred at room temperature for 90 minutes under hydrogen atmosphere (balloon). After filtration through a Celite pad, the filtrate was concentrated in vacuo. The residue was diluted with EtOAc, washed with water and brine, dried over anhydrous MgSO₄ and concentrated in vacuo to give the desired product (18.2 mg, 90%). ¹H-NMR (400 MHz, CDCl₃,) δ 1.65 (8H, m), 2.06 (2H, m), 2.77 (1H, m), 3.09 (3H, s), 4.48 (1H, s), 7.16 (2H, m), 7.70 (2H, d, J=8.4 Hz), 8.01 (2H, d, J=8.4 Hz).

### Example 24

### Preparation of N-(4-(3,5-difluoro-4′-(methylsulfonyl)biphenyl-4-yloxy)cyclohexyl)-3-methylbutanamide

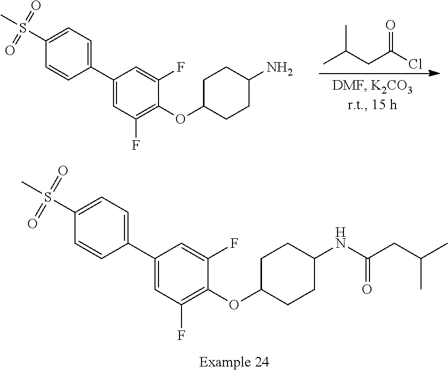

To a stirred solution of 4-(3,5-difluoro-4′-(methylsulfonyl)biphenyl-4-yloxy)cyclohexanamine (57.5 mg, 0.151 mmol) and K₂CO₃ (41.7 mg, 0.301 mmol) in DMF (1.5 ml), was added isovaleroyl chloride (0.020 ml, 0.166 mmol) and the resulting suspension stirred at room temperature for 15 hours. The reaction mixture was extracted with water and EtOAc. The organic layer was washed with brine, dried over anhydrous MgSO₄, filtered and concentrated in vacuo. The residue was purified by column chromatography on SiO₂ to give the desired product (29.2 mg, 41.6%) as a oil. ¹H-NMR (400 MHz, CDCl₃) δ 0.96 (6H, d, J=6.4 Hz), 1.75 (8H, m), 2.09 (3H, m), 3.10 (3H, s), 3.92 (1H, m), 4.49 (1H, s), 5.50 (1H, d, J=8.0 Hz), 7.19 (2H, m), 7.70 (2H, m), 8.02 (2H, m).

### Example 25

### Preparation of 3-chloro-6-(4-(5-ethylpyrimidin-2-yloxy)cyclohexyloxy)pyridazine

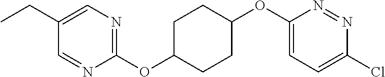

### Step 1. Preparation of 4-(5-ethylpyrimidin-2-yloxy)cyclohexanol

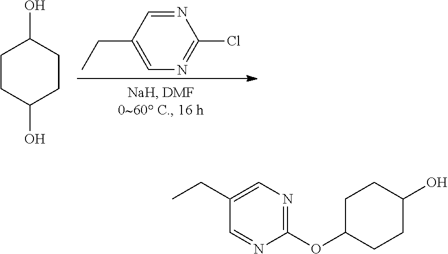

To a stirred solution of cyclohexane-1,4-diol (2.03 g, 17.5 mmol) in DMF (17 mL) was added NaH (95% wt, 177 mg, 7.01 mmol) at 0° C. The mixture was stirred for 30 min at room temperature and 2-chloro-5-ethylpyrimidine (500 mg, 3.51 mmol) was added to the mixture. After being stirred for 16 hours at 70° C., the reaction mixture was diluted with EtOAc and water. The organic layer was washed by brine, dried over Na₂SO₄, filtered and concentrated in vacuo. The residue was purified by column chromatography on SiO₂ (Hex:EtOAc=1:1) to give the desired product (520 mg, 66%) as a colorless oil. ¹H NMR (400 Hz, CDCl₃) δ 1.24 (3H, t, J=7.6 Hz), 1.57-1.84 (5H, m), 2.03-2.20 (3H, m), 2.57 (2H, q, J=7.6 Hz), 3.78-3.82 (1H, m), 4.94-5.01 and 5.06-5.10 (1H, each m), 8.33 (2H, s). LC-MS m/z=222.8 [M+H]⁺.

### Step 2. Preparation of 3-chloro-6-(4-(5-ethylpyrimidin-2-yloxy)cyclohexyloxy)pyridazine

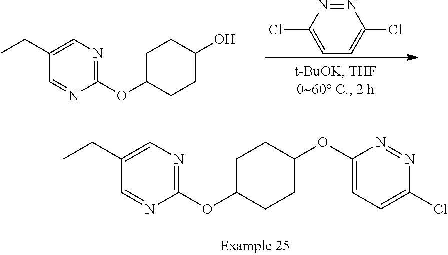

To a solution of 4-(5-ethylpyrimidin-2-yloxy)cyclohexanol (160 mg, 0.720 mmol) in THF (4.80 mL) was added t-BuOK (242 mg, 2.16 mmol) at 0° C. After being stirred for 15 minutes at room temperature, 3,6-dichloropyridazine (214 mg, 1.44 mmol) was added to the mixture at 0° C. The reaction mixture was stirred for 2 hours at 65° C. and diluted with EtOAc and brine. The organic layer was dried over Na₂SO₄, filtered and concentrated in vacuo. The residue was purified by column chromatography on SiO₂ (Hex:EtOAc=2:1 to 1:1) to give the desired product (190 mg, 79%) as a yellow oil. ¹H NMR (400 Hz, CDCl₃) δ 1.25 (3H, t, J=7.6 Hz), 1.12-1.98 (4H, m), 2.07-2.31 (4H, m), 2.58 (2H, q, J=7.6 Hz), 5.06-5.17 (1H, m), 5.35-5.44 (1H, m), 6.93 (1H, dd, J=9.0, 3.4 Hz), 7.36 (1H, d, J=9.0 Hz), 8.34 (2H, s). LC-MS m/z=334.7 [M+H]⁺.

### Example 26

### Preparation of 6-(4-(5-ethylpyrimidin-2-yloxy)cyclohexyloxy)-N-(4-(methylsulfonyl)phenyl)pyridazin-3-amine

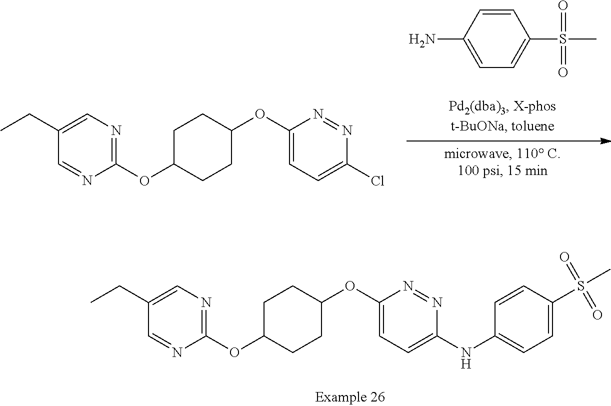

A mixture of 3-chloro-6-(4-(5-ethylpyrimidin-2-yloxy)cyclohexyloxy)pyridazine (70 mg, 0.209 mmol), 4-(methylsulfonyl)aniline (53.7 mg, 0.314 mmol), Pd₂(dba)₃ (19.1 mg, 0.021 mmol), X-phos (19.9 mg, 0.042 mmol) and t-BuONa (40.2 mg, 0.418 mmol) in degassed toluene (0.70 mL) was stirred under microwave at 110° C. for 15 minutes. The reaction mixture was partitioned between EtOAc and water. The organic layer was washed with brine, dried over Na₂SO₄, filtered and concentrated in vacuo. The residue was purified by column chromatography on SiO₂ (Hex:EtOAc=1:2 to 2:1) and further purified by preparative TLC (Hex:EtOAc=1:1) to give the desired product (45 mg, 45%) as a pale yellow oil. ¹H NMR (400 Hz, CDCl₃) δ 8.30 (2H, d, J=6.8 Hz), 7.76-7.70 (4H, m), 7.21-7.10 (1H, m), 6.92-6.86 (1H, m), 5.33-5.25 (1H, m), 5.13-5.04 (1H, m), 2.99 (3H, s), 2.57-2.50 (2H, m), 2.25-1.96 (4H, m), 1.90-1.65 (4H, m), 1.23-1.18 (3H, m). LC-MS m/z=469.9 [M+H]⁺.

### Example 27

### Preparation of 3-fluoro-N-((1R,4R)-4-hydroxycyclohexyl)-4′-(methylsulfonyl) biphenyl-4-carboxamide

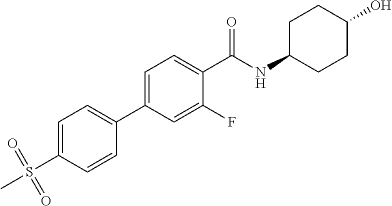

### Step 1. Preparation of ethyl 4-bromo-2-fluorobenzoate

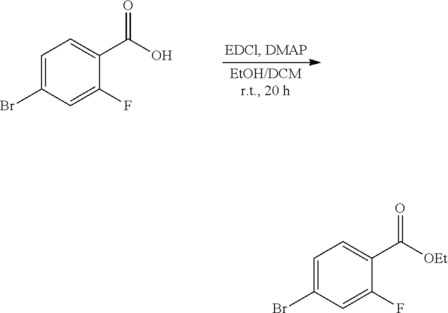

To a stirred solution of 4-bromo-2-fluorobenzoic acid (1.50 g, 6.85 mmol) in DCM (26.0 mL) was added EDCI (1.60 g, 8.35 mmol), DMAP (130 mg, 1.06 mmol), and EtOH (2.00 mL) at room temperature. After being stirred for 20 hours at room temperature, the reaction mixture was washed with water and brine. The organic phase was dried over Na₂SO₄, filtered and concentrated in vacuo. The residue was purified by column chromatography on SiO₂ (Hexanes/EtOAc=1/5) to give the desired product (1.52 g, 90%) as a colorless oil. ¹H-NMR (400 MHz, CDCl₃) δ 1.39 (3H, t, J=6.8 Hz), 4.39 (2H, q, J=6.8 Hz), 7.32-7.37 (2H, m), 7.80-7.84 (1H, m).

### Step 2. Preparation of ethyl 3-fluoro-4′-(methylsulfonyl)biphenyl-4-carboxylate

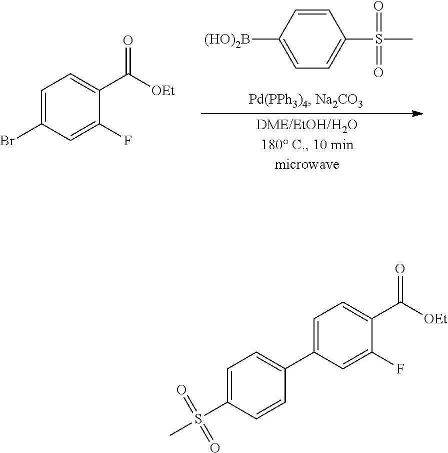

According to the similar procedure of Example 40, the desired product was obtained as a white solid. ¹H-NMR (400 MHz, CDCl₃) δ 1.43 (3H, t, J=6.8 Hz), 3.11 (3H, s), 4.44 (2H, q, J=6.8 Hz), 7.37-7.77 (2H, m), 7.78-7.81 (2H, m), 8.04-8.08 (3H, m).

### Step 3. Preparation of 3-fluoro-4′-(methylsulfonyl)biphenyl-4-carboxylic acid

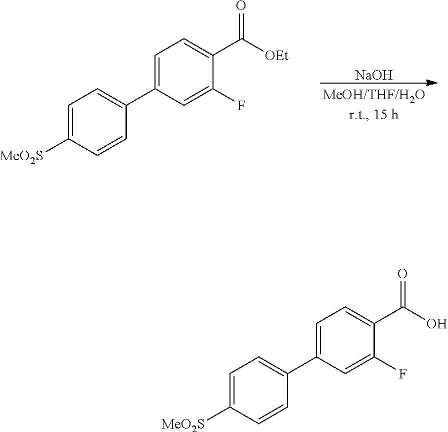

To a stirred solution of ethyl 3-fluoro-4′-(methylsulfonyl)biphenyl-4-carboxylate (290 mg, 0.900 mmol) in MeOH (1.50 mL), THF (1.50 mL) and water (1.50 mL) was added sodium hydroxide (180 mg, 4.50 mmol) at room temperature. After being stirred for 15 hours at room temperature, the reaction mixture was cooled to 0° C. and acidified with citric acide until pH=3 to yield a white precipitate. The solid was collected by filtration and washed with cold water and dried in vacuo to give the desired compound (223 mg, 84%) as a white solid. ¹H-NMR (400 MHz, CDCl₃) δ 1.43 (3H, t, J=6.8 Hz), 3.11 (3H, s), 4.44 (2H, q, J=6.8 Hz), 7.39 (1H, dd, J=11.6, 2.0 Hz), 7.45 (1H, dd, J=8.4, 2.0 Hz), 7.78-7.81 (2H, m), 8.04-8.08 (3H, m).

### Step 4. Preparation of 3-fluoro-N-((1R,4R)-4-hydroxycyclohexyl)-4′-(methylsulfonyl) biphenyl-4-carboxamide

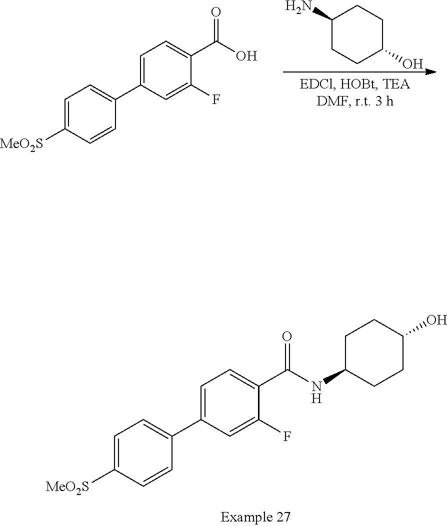

To a stirred solution of compound 3-fluoro-4′-(methylsulfonyl)biphenyl-4-carboxylic acid (86.0 mg, 0.292 mmol) in DMF (3.00 mL) was added EDCI (80.0 mg, 0.417 mmol) and HOBt (60.0 mg, 0.444 mmol), TEA (0.12 mL, 0.861 mmol) and (1R,4R)-4-aminocyclohexanol (70.0 mg, 0.608 mmol) at room temperature. After being stirred for 12 hours at room temperature, the reaction mixture was diluted with CHCl₃ and washed with water and brine, dried over Na₂SO₄, filtered and concentrated in vacuo. The residue was purified by recrystallization from Hexanes/CHCl₃ mixture to give the desired product (63.0 mg, 55%) as a white solid. ¹H-NMR (400 MHz, DMSO-d₆) δ 1.24-1.35 (4H, m), 1.83-1.86 (4H, m), 2.99-3.05 (1H, m), 3.27 (3H, s), 3.69-3.71 (1H, m), 4.58 (1H, d, J=4.4 Hz), 7.63-7.69 (2H, m), 7.72-7.75 (1H, m), 8.03 (4H, s), 8.23-8.25 (1H, m). LC-MS Calcd. 391.13, Found: 391.94 [M+H]⁺.

### Example 28

### Preparation of N-(4-(5-ethylpyrimidin-2-yloxy)cyclohexyl)-3-fluoro-4′-(methylsulfonyl)biphenyl-4-carboxamide

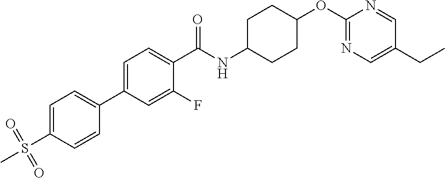

### Step 1. Preparation of 4-(5-ethylpyrimidin-2-yloxy)cyclohexanamine

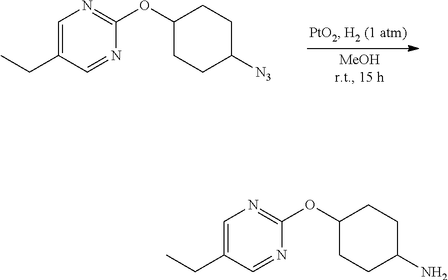

A stirred solution of 2-(4-azidocyclohexyloxy)-5-ethylpyrimidine (350 mg, 1.42 mmol) in MeOH (3.50 mL) was hydrogenated over PtO₂ (35.0 mg, 0.154 mmol) at room temperature for 15 hours. The mixture was filtered through a pad of Celite and the filtrate concentrated in vacuo to give the desired product which was used without further purification.

### Step 2. Preparation of N-(4-(5-ethylpyrimidin-2-yloxy)cyclohexyl)-3-fluoro-4′-(methylsulfonyl)biphenyl-4-carboxamide

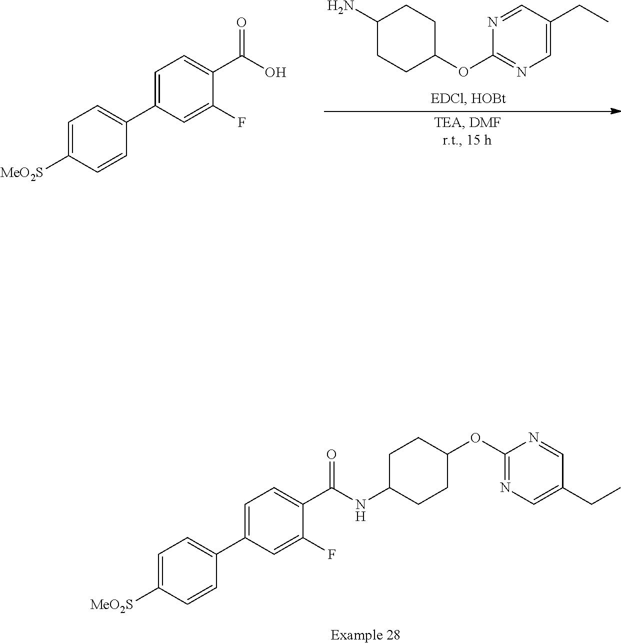

To a stirred solution of 3-fluoro-4′-(methylsulfonyl)biphenyl-4-carboxylic acid (120 mg, 0.408 mmol) in DMF (3.00 mL) was added EDCI (94.0 mg, 0.490 mmol), HOBt (66.0 mg, 0.488 mmol), TEA (0.15 mL, 1.08 mmol) and then 4-(5-ethylpyrimidin-2-yloxy)cyclohexanamine (150 mg, 0.678 mmol) at room temperature. After being stirred for 15 hours at room temperature, the reaction mixture was diluted with water and brine and extracted with CHCl₃. The organic phase was dried over Na₂SO₄, filtered and concentrated in vacuo. The residue was purified by column chromatography on SiO₂ (EtOAc only) and then recrystallized from Hexanes/EtOAc mixture to give the desired compound, Example 28 (143 mg, 71%) as a white solid. ¹H-NMR (400 MHz, DMSO-d₆) δ 3.28 (3H, s), 7.72 (1H, dd, J=8.0, 1.6 Hz), 7.78 (1H, dd, J=12.0, 1.6 Hz), 7.99 (1H, t, J=8.0 Hz), 8.02-8.07 (4H, m), 13.4 (1H, brs). LC-MS m/z=498.2 [M+H]⁺.

### Example 29

### Preparation of (1R,4R)-4-(3-fluoro-4′-(methylsulfonyl)biphenyl-4-ylcarboxamido) cyclohexyl 3-methylbutanoate

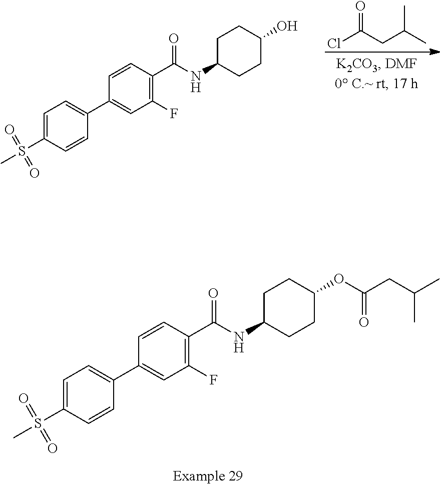

To a stirred solution of 3-fluoro-N-((1R,4R)-4-hydroxycyclohexyl)-4′-(methylsulfonyl) biphenyl-4-carboxamide (55.0 mg, 0.141 mmol) in dry DMF (1.50 mL) was added potassium carbonate (30.0 mg, 0.217 mmol) at room temperature. After being stirred for 30 min at room temperature, 3-methylbutanoyl chloride (0.100 mL, 0.796 mmol) was added to the mixture at 0 C. The mixture was stirred for 17 hours at room temperature, diluted with water and extracted with EtOAc. The separated organic layers were washed with brine, dried over anhydrous Na₂SO₄, filtered and concentrated in vacuo. The residue was purified by column chromatography on SiO₂ (Hexanes/EtOAc=1:1 to 1:3) to give the desired product (28.0 mg, 42%) as a yellow solid. ¹H NMR (400 MHz, DMSO-d₆) δ 0.904 (6H, d, J=8.8 Hz), 1.21-1.24 (1H, m), 1.42-1.46 (4H, m), 1.92-2.00 (4H, m), 2.15-2.17 (1H, m), 2.80-2.95 (1H, m), 3.27 (3H, s), 3.72-3.79 (1H, m), 4.60-4.70 (1H, m), 7.67-7.76 (3H, m), 8.03 (4H, s), 8.31 (1H, d, J=8.0 Hz). LC-MS Calcd. 475.18, Found: 475.69 [M+H]⁺.

### Example 30

### Preparation of 1-(4-(benzyloxy)cyclohexyloxy)-4-bromo-2-fluorobenzene

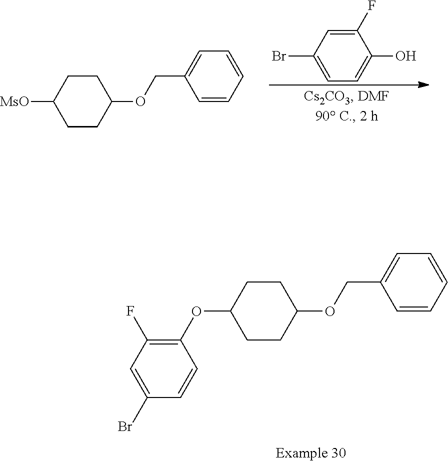

To a stirred solution of 4-(benzyloxy)cyclohexyl methanesulfonate (1.00 g, 3.52 mmol) in dry DMF, (14.0 mL) was added 4-bromo-2-fluorophenol (0.420 mL, 3.87 mmol) and Cs₂CO₃ (1.80 g, 5.52 mmol) at room temperature. After being stirred for 2 hours at 90° C., the reaction mixture was diluted with water and extracted with EtOAc. The separated organic layer was washed with brine, dried over anhydrous Na₂SO₄, filtered and concentrated in vacuo. The residue was purified by column chromatography on SiO₂ (Hexanes:EtOAc=1:1) to give the compound 8 (690 mg, 52%) as a pale yellow solid. ¹H-NMR (400 MHz, CDCl₃) δ 1.48-1.73 (4H, m), 1.90-2.05 (4H, m), 3.50-3.54 (1H, m), 4.24-4.32 (1H, m), 4.57 (2H, s), 6.84-6.89 (1H, m), 7.14-7.18 (1H, m), 7.27-7.30 (1H, m), 7.32-7.37 (5H, m).

### Example 31

### Preparation of 4-(4-(benzyloxy)cyclohexyloxy)-3-fluoro-4′-(methylsulfonyl)biphenyl

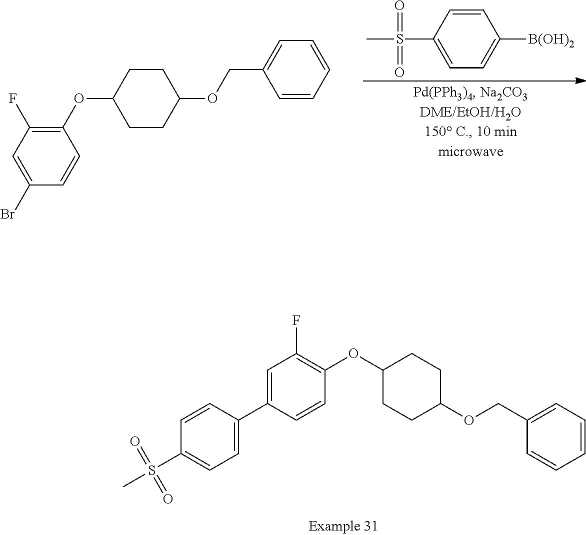

To a stirred solution of 1-(4-(benzyloxy)cyclohexyloxy)-4-bromo-2-fluorobenzene (690 mg, 1.82 mmol) in dry DME (8.00 mL), EtOH (2.00 mL) and water (2.00 mL) was added 4-(methylsulfonyl)phenylboronic acid (437 mg, 2.19 mmol), Na₂CO₃ (578 mg, 5.45 mmol) and Pd(PPh₃)₄ (63.0 mg, 0.0550 mmol) at room temperature. After being irradiated for 10 minutes at 150° C. in a microwave, the reaction mixture was cooled and filtered through Celite and the filtrate was concentrated in vacuo. The residue was extracted with EtOAc/water and the combined organic layers were washed with brine, dried over anhydrous Na₂SO₄, filtered and concentrated in vacuo. The residue was purified by column chromatography on SiO₂ (Hexanes:EtOAc=3:1 to 1:1) to give the desired product (360 mg, 44%) as a white solid. ¹H-NMR (400 MHz, CDCl₃) δ 1.65-1.78 (4H, m), 1.97-2.14 (4H, m), 3.09 (3H, s). 3.53-3.57 (1H, m), 4.42-4.45 (1H, m), 4.57 (2H, s), 7.09 (1H, t, J=8.4 Hz), 7.27-7.37 (7H, m), 7.71 (2H, d, J=8.4 Hz), 7.99 (2H, d, J=8.4 Hz).

### Example 32

### Preparation of (1R,3S,5S)-tert-butyl 3-(6-(4-ethylcyclohexyloxy)pyridazin-3-yloxy)-8-azabicyclo[3.2.1]octane-8-carboxylate

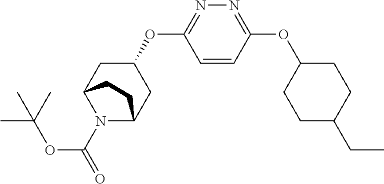

### Step 1. Preparation of (1R,5S)-tert-butyl 3-oxo-8-azabicyclo[3.2.1]octane-8-carboxylate

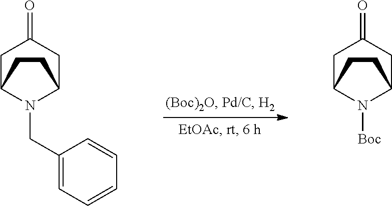

A suspension of (1R,5S)-8-benzyl-8-azabicyclo[3.2.1]octan-3-one (3.00 g, 13.9 mmol), (Boc)₂O (3.88 mL, 16.7 mmol) and Pd/C (0.148 g, 1.39 mmol) in EtOAc (34 mL) was stirred at room temperature under hydrogen atmosphere (balloon) for 6 hours. The reaction mixture was filtered through a Celite pad and concentrated in vacuo to give the desired product (3.14 g, 100%) as a white solid, which was used for the next step without further purification. ¹H-NMR (400 MHz, CDCl₃) δ 1.50 (9H, s), 1.66 (2H, d), 2.10 (2H, m), 2.36 (2H, d), 2.72 (2H, br), 4.50 (2H, s).

### Step 2. Preparation of (1R,3R,5S)-tert-butyl 3-hydroxy-8-azabicyclo[3.2.1]octane-8-carboxylate and (1R,3S,5S)-tert-butyl 3-hydroxy-8-azabicyclo[3.2.1]octane-8-carboxylate

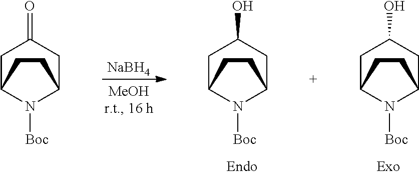

To a stirred solution of (1R,5S)-tert-butyl 3-oxo-8-azabicyclo[3.2.1]octane-8-carboxy late (3.14 g, 13.9 mmol) in MeOH (25 mL) was added sodium borohydride (0.791 g, 20.9 mmol) portion wise at 0° C. The ice bath was removed and the stirring continued for 16 hours at room temperature. The mixture was diluted with DCM and washed with water. The organic phase was dried over Na₂SO₄, filtered and concentrated in vacuo. The residue was purified by column chromatography on SiO₂ (Hex:EtOAc=3:1) to give the (1R,3R,5S)-tert-butyl 3-hydroxy-8-azabicyclo[3.2.1]octane-8-carboxylate, endo product (1.50 g, 47.3%) and (1R,3S,5S)-tert-butyl 3-hydroxy-8-azabicyclo[3.2.1]octane-8-carboxylate, exo product (1.18 g, 37.2%) as a white solid each.

Endo isomer; (1R,3R,5S)-tert-butyl 3-hydroxy-8-azabicyclo[3.2.1]octane-8-carboxylate

¹H-NMR (400 MHz, CDCl₃) δ 1.46 (9H, s), 1.69 (2H, d), 1.94-2.16 (6H, m), 4.14-4.22 (3H, m).

Exo isomer; (1R,3S,5S)-tert-butyl 3-hydroxy-8-azabicyclo[3.2.1]octane-8-carboxylate

¹H-NMR (400 MHz, CDCl₃) δ 1.47 (9H, s). 1.56-1.62 (4H, m), 1.95 (4H, m), 4.11-4.28 (3H, m).

### Step 3. Preparation of (1R,3S,5S)-tert-butyl 3-(6-chloropyridazin-3-yloxy)-8-azabicyclo[3.2.1]octane-8-carboxylate

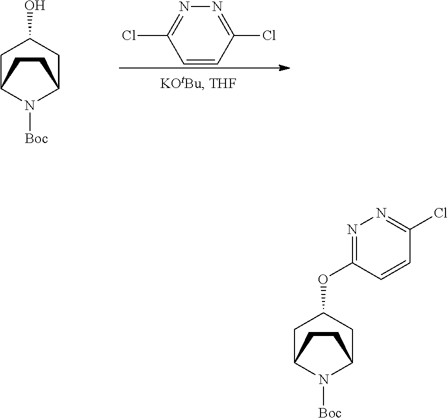

A stirred mixture of 3,6-dichloropyridazine (50 mg, 0.336 mmol), (1R,3S,5S)-tert-butyl 3-hydroxy-8-azabicyclo[3.2.1]octane-8-carboxylate (76 mg, 0.336 mmol) and potassium tert-butoxide (28.6 mg, 0.403 mmol) in THF (2 ml) was heated at 60° C. for 24 hours. The reaction mixture was filtered through a Celite pad and the filtrate was concentrated under reduced pressure. The reaction mixture was partitioned between EtOAc and water. The organic layer was washed with brine, dried over Na₂SO₄, filtered and concentrated in vacuo. The residue was purified by column chromatography on SiO₂ (Hex: EtOAc=10:1) to give the desired product (50 mg, 44%) as a white solid.

### Step 4. Preparation of (1R,3S,5S)-tert-butyl 3-(6-(4-ethylcyclohexyloxy)pyridazin-3-yloxy)-8-azabicyclo[3.2.1]octane-8-carboxylate

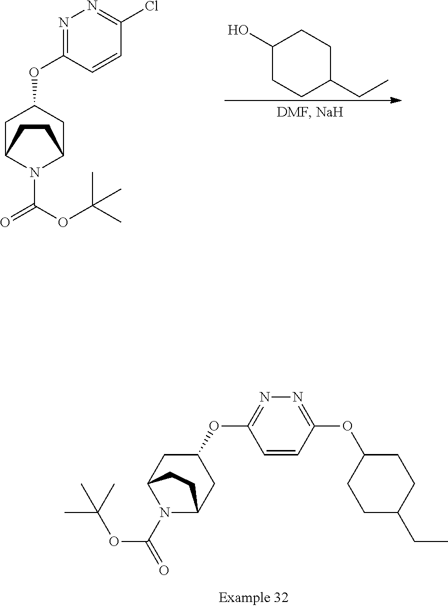

According to the similar procedure of step 1 in Example 25, the desired product was obtained. ¹H-NMR (400 MHz, CDCl₃) δ 0.89 (311, t, J=7.6 Hz), 1.05-1.11 (2H, m), 1.16-1.28 (4H, m), 1.49 (9H, s), 1.81 (5H, m), 1.98 (2H, m), 2.22 (4H, m), 4.24 (1H, m). 4.33 (1H, m), 5.10 (1H, m), 5.62 (1H, m), 6.83 (2H, d, J=1.6 Hz). LC-MS Calcd. 431.28, Found 432.05 [M+H]⁺.

### Example 33

### Preparation of (1R,3S,5S)-3-(6-(4-ethylcyclohexyloxy)pyridazin-3-yloxy)-8-(methyl sulfonyl)-8-azabicyclo[3.2.1]octane

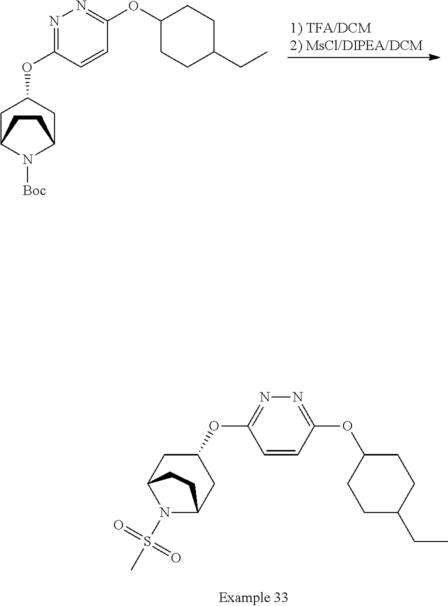

To a stirred solution of (1R,3S,5S)-tert-butyl 3-(6-(4-ethylcyclohexyloxy)pyridazin-3-yloxy)-8-azabicyclo[3.2.1]octane-8-carboxylate (14 mg, 0.032 mmol) in DCM (1.5 mL) was added TFA (1.5 mL) at room temperature and the stirring continued for 1 hour at room temperature. The solvent was completely removed in vacuo and the intermediate amine was dissolved in DCM (1.5 mL). To a stirred solution of this intermediate amine was added DIPEA (0.056 mL, 0.32 mmol) at 0° C. Methanesulfonyl chloride (0.0074 mL, 0.096 mmol) in DCM (0.5 mL) was added dropwise at 0° C. and the mixture was stirred for 30 minutes at 0° C. The mixture was quenched with MeOH (0.01 mL) in DCM (0.5 mL) and the solvents was removed under reduced pressure. The residue was purified by Prep TLC to give the desired product (11 mg, 83%) as a white solid. ¹H-NMR (400 MHz, CDCl₃) δ 0.89 (3H, t, J=7.2 Hz), 1.08 (2H, m), 1.23 (4H, m), 1.40 (2H, m), 1.82-1.96 (5H, m), 2.10 (2H, m), 2.22 (2H, m), 2.37 (2H, m), 2.95 (3H, s), 4.34 (2H, m), 5.09 (1H, m), 5.53 (1H, m), 6.84 (2H, d, J=2.8 Hz). LC-MS Calcd. 409.20, Found 410.01 [M+H]⁺.

### Example 34

### Preparation of 2-(4-(5-ethylpyrimidin-2-yloxy)cyclohexyl)-4-(((1R,3S,5S)-8-(methylsulfonyl)-8-azabicyclo[3.2.1]octan-3-yloxy)methyl)thiazole

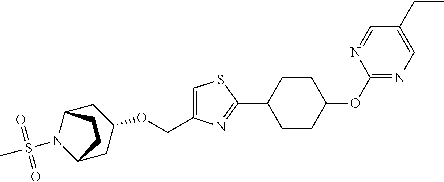

### Step 1. Preparation of (1R,3S,5S)-8-azabicyclo[3.2.1]octan-3-ol (hydrochloride)

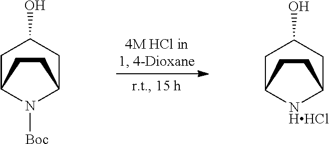

A mixture of (1R,3S,5S)-tert-butyl 3-hydroxy-8-azabicyclo[3.2.1]octane-8-carboxylate (300 mg, 1.32 mmol) and 4 M HCl in 1,4-dioxane (4.0 mL) was stirred at room temperature for 15 hours. The reaction mixture was concentrated in vacuo to give the desired product (168 mg, quant) as a white solid, which was used for the next step without further purification. LC-MS Calcd. 127.1, Found 127.90 [M+H]⁺

### Step 2. (1R,3S,5S)-8-(methylsulfonyl)-8-azabicyclo[3.2.1]octan-3-ol

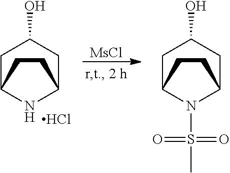

According to the similar procedure in Example 33, the desired product was obtained. ¹H-NMR (400 MHz, CDCl₃) δ 1.72 (4H, m), 2.05 (4H, m), 2.94 (3H, s), 4.04 (1H, m), 4.28 (2H, m).

### Step 3. Preparation of 2-(4-(5-ethylpyrimidin-2-yloxy)cyclohexyl)-4-(((1R,3S,5S)-8-(methylsulfonyl)-8-azabicyclo[3.2.1]octan-3-yloxy)methyl)thiazole

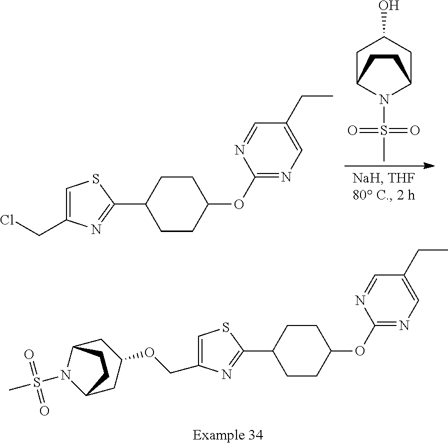

According to the similar procedure in Example 12, the desired product was obtained. ¹H-NMR (400 MHz, CDCl₃) δ 1.24 (3H, t), 1.70-2.34 (16H, m), 2.59 (2H, q), 2.94 (3H, s), 3.84 (1H, m), 4.01 (1H, m), 4.28 (2H, m), 4.60 (2H, m), 5.02, 5.30 (1H, m), 7.07 (1H, m), 8.33 (2H, m). LC-MS Calcd. 506.2, Found 506.78 [M+H]⁺.

### Example 35

### Preparation of 2-(4-(5-ethylpyrimidin-2-yloxy)cyclohexyl)-4-(((1R,3r,5S)-8-(methylsulfonyl)-8-azabicyclo[3.2.1]octan-3-yloxy)methyl)thiazole (Endo product)

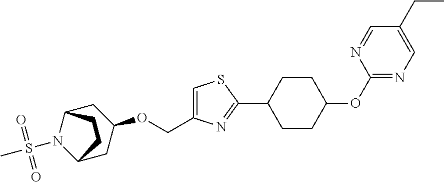

### Step 1. Preparation of (1R,3R,5S)-8-azabicyclo[3.2.1]octan-3-ol (hydrochloride)

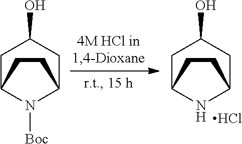

According to the similar procedure of step 1 in Example 34, the desired product was obtained. LC-MS Calcd. 127.1, Found 127.92 [M+H]⁺.

### Step 2. Preparation of (1R,3R,5S)-8-(methylsulfonyl)-8-azabicyclo[3.2.1]octan-3-ol

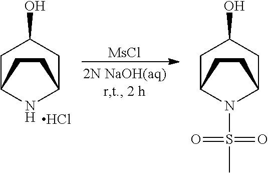

To stirred solution of (1R,3R,5S)-8-azabicyclo[3.2.1]octan-3-ol hydrochloride (560 mg, 4.4 mmol) in 2M NaOH (22.0 mL, 44.0 mmol) was added methanesulfonyl chloride (0.55 mL, 7.04 mmol) over 10 minutes. The solution was stirred at room temperature for a further 15 hours. The reaction mixture was poured into EtOAc and the organic phase separated. The aqueous phase was extracted with EtOAc. The combined organic layers were dried over MgSO₄, filtered and concentrated in vacuo. The residue was purified by column chromatography on SiO₂ (Hexanes/EtOAc=2:1) to give the desired product (673 mg, 74.5%) as a white solid. ¹H-NMR (400 MHz, CDCl₃) δ 1.86 (2H, m), 2.01 (2H, m), 2.18 (2H, m), 2.29 (2H, m), 2.88 (3H, s), 4.14 (1H, m), 4.22 (2H, m).

### Step 3. Preparation of 2-(4-(5-ethylpyrimidin-2-yloxy)cyclohexyl)-4-(41R,3R,5S)-8-(methylsulfonyl)-8-azabicyclo[3.2.1]octan-3-yloxy)methyl)thiazole

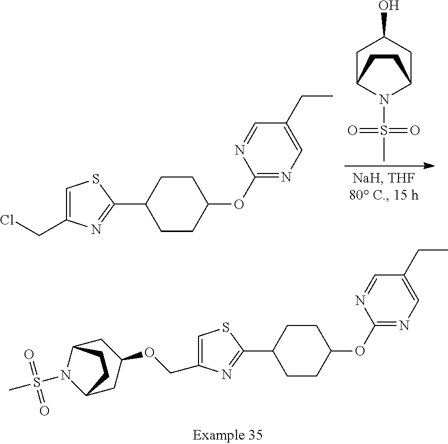

According to the similar procedure in Example 12, the desired product was obtained. ¹H-NMR (400 MHz, CDCl₃) δ 1.24 (3H, t), 1.71 (2H, m), 1.98-2.33 (14H, m), 2.57 (2H, q), 2.88 (3H, s), 3.10 (1H, m), 3.76 (1H, m), 4.22 (2H, s), 4.57 (2H, s), 5.01, 5.29 (1H, m), 7.03 (1H, m), 8.33 (2H, m). LC-MS Calcd. 506.2, Found 506.82 [M+H]⁺.

### Example 36

### Preparation of 5-ethyl-2-(4-(4-(4-(methylsulfonyl)phenoxy)butoxy)cyclohexyloxy)pyrimidine

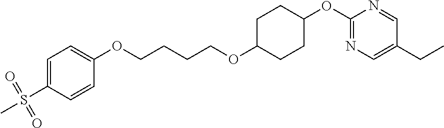

### Step 1. Preparation of 1-(4-bromobutoxy)-4-(methylsulfonyl)benzene

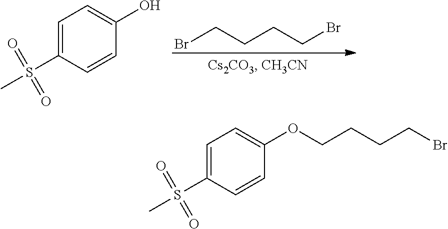

A stirred mixture of 4-(methylsulfonyl)phenol (200 mg, 1.16 mmol), 1,4-dibromobutane (2.5 g, 11.61 mmol) and cesium carbonate (390 mg, 3.48 mmol) in acetonitrile (10 mL) was heated to 90° C. for 2 hours. The mixture was cooled to room temperature and concentrated in vacuo. The residue was purified by column chromatography on SiO₂ (Hex:EtOAc=3:1 to 1:1) to give the desired product (280 mg, 78%) as a colorless oil. ¹H NMR (400 MHz, CDCl₃) δ 1.98-2.10 (4H, m), 3.03 (3H, s), 3.50 (2H, t, J=6.4 Hz), 4.08 (2H, t, J=6.0 Hz), 7.01 (2H, d, J=8.8 Hz), 7.86 (2H, d, J=9.2 Hz).

### Step 2. Preparation of 5-ethyl-2-(4-(4-(4-(methylsulfonyl)phenoxy)butoxy)cyclohexyloxy)pyrimidine

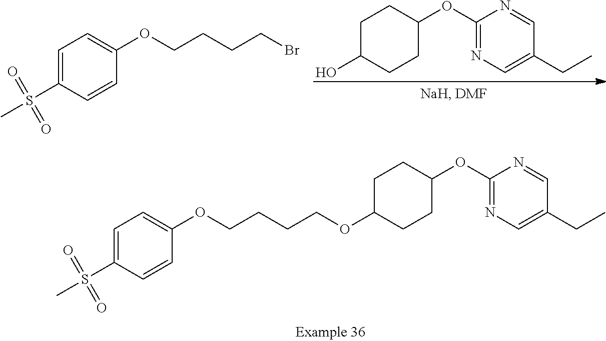

To a stirred solution of 4-(5-ethylpyrimidin-2-yloxy)cyclohexanol (120 mg, 0.54 mmol) in anhydrous DMF (1.5 mL) was added NaH (20.5 mg, 0.81 mmol) at room temperature. This mixture was stirred for 10 minutes at room temperature and added to the stirred solution of 1-(4-bromobutoxy)-4-(methylsulfonyl)benzene in anhydrous DMF (1.5 mL) dropwise at room temperature. The mixture was stirred for 3 hours at room temperature. The solvent was removed in vacuo and the residue was purified by column chromatography on SiO₂ (Hex: EtOAc=2:1 to 1:2) to give the desired product (80 mg, 33%) as a colorless oil. ¹H NMR (400 MHz, CDCl₃) δ 1.24 (3H, t, J=7.6 Hz), 1.43-2.17 (12H, m), 2.56 (2H, q, J=7.6 Hz), 3.03 (3H, s), 3.40 (1H, m), 3.53 (2H, m), 4.08 (2H, m), 5.02 (1H, m), 7.01 (2H, m), 7.86 (2H, m), 8.33 (2H, s). LC-MS m/z=449.1 [M+H]⁺.

### Example 37

### Preparation of 2-(3-(benzyloxy)cyclohexyloxy)-5-bromo-1,3-difluorobenzene

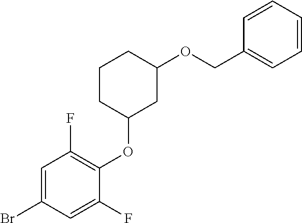

### Step 1. Preparation of 3-(benzyloxy)cyclohexanol (cis, trans mixture)

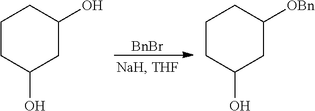

To a stirred solution of cyclohexane-1,3-diol (2.72 g, 23.4 mmol) in THF (30 mL) was added NaH (590 mg, 23.4 mmol) in small portions at 0° C. The mixture was stirred for 20 minutes at room temperature. Benzyl bromide (1.39 mL, 11.7 mmol) was added. The mixture was stirred for 3 hours at 50° C. DMSO (10 mL) was added and the stirring continued for 2 hours at 60° C. The mixture was cooled to room temperature and extracted with aq. 1 N HCl/EtOAc (150 mL/200 mL×2). The combined organic layers were dried over MgSO₄, filtered, concentrated, and purified by column chromatography on SiO₂ (Hex:ethyl acetate=3/1) to give the desired product (1.3 g, 54%) as a colorless oil. ¹H NMR (400 MHz, CDCl₃) δ 1.23-2.09 (16H, m), 3.53 (1H, m), 3.70 (1H, m), 3.80 (1H, m), 4.06 (1H, m), 4.48-4.59 (4H, m), 7.25-7.34 (10H, m). LC-MS Calcd. 206.13, Found 206.88 [M+H]⁺.

### Step 2. Preparation of 2-(3-(benzyloxy)cyclohexyloxy)-5-bromo-1,3-difluorobenzene (cis, trans mixture)

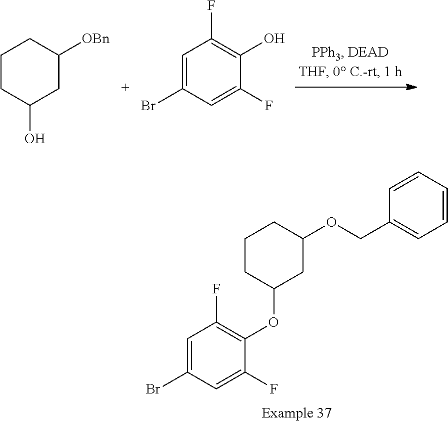

To a stirred solution of 3-(benzyloxy)cyclohexanol (200 mg, 0.970 mmol), 4-bromo-2,6-difluorophenol (223 mg, 1.067 mmol), triphenylphosphine (509 mg, 1.939 mmol), triethylamine (149 μl, 1.067 mmol) in THF (15 mL) was added diethyl azodicarboxylate (305 μl, 1.939 mmol) dropwise at 0° C. The mixture was stirred for 1 hour at room temperature and was concentrated. The residue was purified by column chromatography on SiO₂ to give the desired product (200 mg, 52%) as a colorless oil. NMR (400 MHz, CDCl₃) δ 1.14-2.08 (16H, m), 2.51 (1H, m), 3.33 (1H, m), 3.87 (1H, m), 3.99 (1H, m), 4.48-4.53 (4H, m). 7.05-7.10 (4H, m), 7.25-7.34 (10H, m).

### Example 38

### Preparation of 4-(3-(benzyloxy)cyclohexyloxy)-3,5-difluoro-4′-(methylsulfonyl)biphenyl (cis, trans mixture)

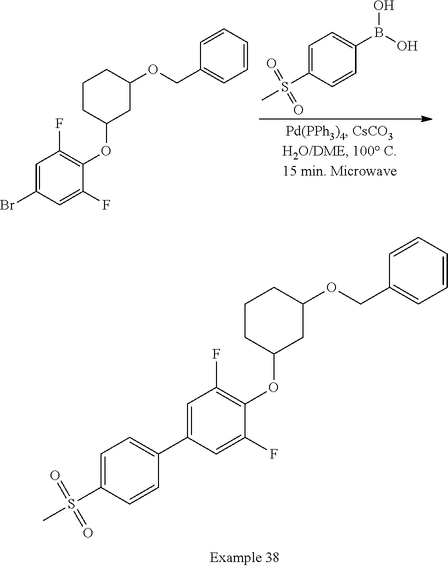

A mixture of 2-(3-(benzyloxy)cyclohexyloxy)-5-bromo-1,3-difluorobenzene (150 mg, 0.378 mmol), 4-(methylsulfonyl)phenylboronic acid (83 mg, 0.415 mmol), cesium carbonate (369 mg, 1.133 mmol) and Pd(Ph₃P)₄ (44 mg, 0.038 mmol) in water (1.5 ml), and dimethoxyethane (1.5 ml) was subjected to microwave irradiation (100° C., 15 minutes). After being cooled to room temperature, the reaction mixture was extracted with EtOAc/H₂O. The organic phase was separated, dried (MgSO₄), filtered, concentrated and the residue was purified by chromatography on SiO₂ (hexanes:EtOAc=3:1) to give the desired product as colorless oil. ¹H NMR (400 MHz, CDCl₃) δ 1.16-2.14 (16H, m), 2.58 (1H, m), 3.06-4.00 (6H, m), 3.37 (1H, m), 3.92 (1H, m), 4.12 (1H, m), 4.51-4.62 (4H, m), 7.14-7.20 (4H, m), 7.24-7.34 (10H, m), 7.69-7.71 (4H, m), 8.00-8.02 (4H, m).

### Example 39

### Preparation of 3-(3,5-difluoro-4′-(methylsulfonyl)biphenyl-4-yloxy)cyclohexanol (cis, trans mixture)

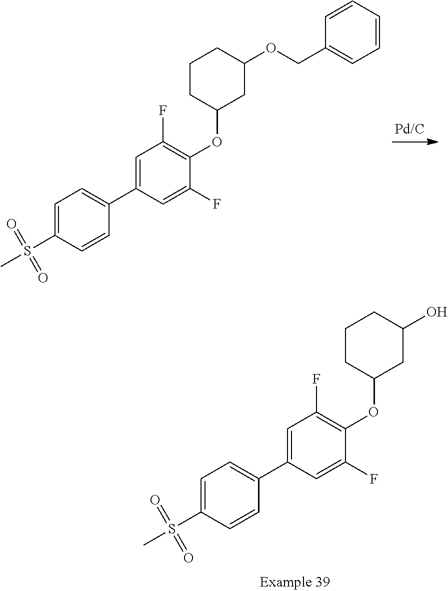

To a stirred solution of 4-(3-(benzyloxy)cyclohexyloxy)-3,5-difluoro-4′-(methylsulfonyl)biphenyl (200 mg, 0.423 mmol) in dichloromethane/MeOH (2 mL/2 mL) was added 5% Pd/C (100 mg) carefully. The mixture was hydrogenated overnight at room temperature (balloon). After completion of the reaction, the mixture was filtered and the filtrate was concentrated to give the desired product as a white solid. ¹H NMR (400 MHz, CDCl₃) δ 1.25-2.15 (16H, m), 2.34 (1H, m), 3.10 (6H, s), 3.74 (1H, s), 4.20-4.67 (4H, m), 7.18 (4H, m), 7.70 (4H, m), 8.01 (4H, m).

### Example 40

### Preparation of 2-(4-(3,5-difluoro-4′-(methylsulfonyl)biphenyl-4-yloxy)cyclo hexyloxy)-5-ethylpyrimidine

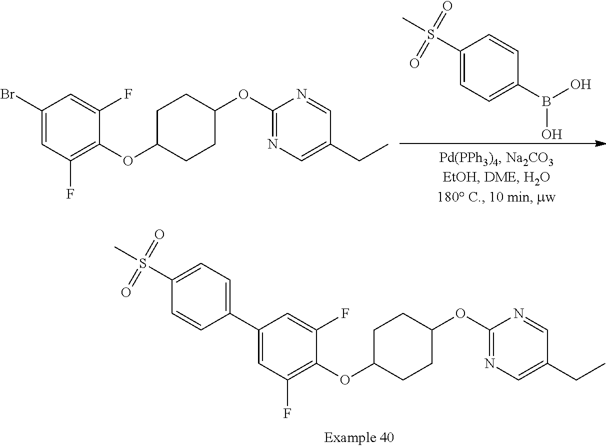

A mixture of 2-(4-(4-bromo-2,6-difluorophenoxy)cyclohexyloxy)-5-ethylpyrimidine (58.9 mg, 0.143 mmol), 4-(methylsulfonyl)phenylboronic acid (34.2 mg, 0.171 mmol), Na₂CO₃ (45.3 mg, 0.428 mmol) and Pd(Ph₃P)₄ (16.5 mg, 0.014 mmol) in H₂O (0.24 mL), EtOH (0.36 mL) and DME (0.72 mL) was subjected to microwave irradiation (180° C., 10 min). After being cooled to room temperature, the reaction mixture was extracted with EtOAc and Water. The organic layer was separated, dried (MgSO₄), and purified by flash chromatography to give the desired product (41.2 mg, 59%) as a yellow solid. ¹H-NMR (400 MHz, CDCl₃) δ 1.24 (3H, t, J=8.8 Hz), 1.84 (4H, m), 2.16 (4H, m), 2.57 (2H, q), 3.09 (3H, s), 4.21 (1H, m), 5.07-5.17 (1H, m), 7.16 (2H, m), 7.70 (2H, d, J=8.4 Hz), 8.01 (2H, d, J=6.8 Hz), 8.34 (2H, m). LC-MS Calcd. 488.16, Found 489.02 [M+H]⁺.

### Example 41

### Preparation of 3-(3,5-difluoro-4′-(methylsulfonyl)biphenyl-4-yloxy)cyclohexyl 3-methyl butanoate (cis, trans mixture)

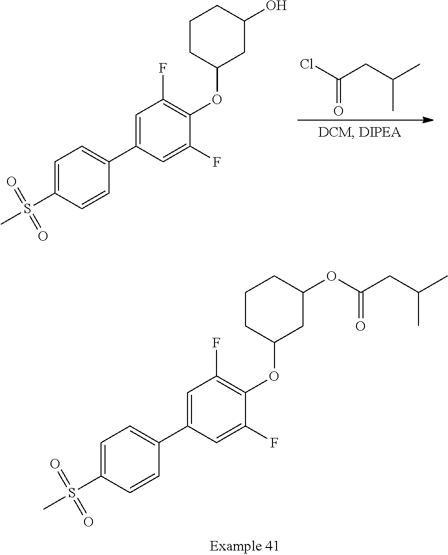

To a stirred solution of 3-(3,5-difluoro-4′-(methylsulfonyl)biphenyl-4-yloxy)cyclohexanol (60 mg, 0.157 mmol) in DCM (2 mL) was added DIPEA (0.055 mL, 0.314 mmol). Isovaleroyl chloride (0.04 mL, 0.314 mmol) was added at room temperature and the stirring continued for 1 hour. DMAP (19 mg, 0.157 mmol) was then added. The mixture was stirred overnight at room temperature. The solvent was removed in vacuo and the residue was extracted by EtOAc/aq. 0.5 M HCl (=50 mL×2/20 mL). The combined organic layers were dried over MgSO₄, filtered and concentrated. The residue was purified by Prep TLC to give the desired product (54 mg, 74%) as pale yellow oil. ¹H NMR (400 MHz, CDCl₃) δ 0.95 (12H, d, J=6.8 Hz), 1.28-2.18 (21H, m), 2.44 (1H, m), 3.10 (6H, s), 4.18 (1H, m), 4.55 (1H, m), 4.74 (1H, m), 5.28 (1H, m), 7.16 (4H, d, J=8.8 Hz), 7.70 (4H, d, J=8.4 Hz), 8.01 (4H, d, J=8.4 Hz).

### Example 42

### Preparation of 4-((1S,4S)-4-ethylcyclohexyloxy)pyridine (cis isomer) and 4-((1R,4R)-4-ethyl cyclohexyloxy)pyridine (trans isomer)

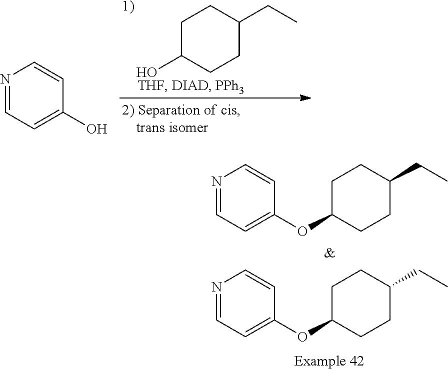

To a stirred solution of 4-hydroxypyridine (100 mg, 1.052 mmol) in anhydrous THF (5 mL) was added 4-ethylcyclohexanol (148 mg, 1.157 mmol) and PPh₃ (413 mg, 1.578 mmol). DIAD (0.31 mL, 1.578 mmol) was added dropwise at room temperature. The mixture was stirred for 4 hours at 65° C. The mixture was cooled to room temperature and the solvent was evaporated. The residue was purified by column chromatography (EA/Hex=3/1) to give 4-(4-ethylcyclohexyloxy)pyridine (125 mg, 58%, cis, trans combined yield) as a colorless oil. The mixture was further purified by preparative TLC (EA/Hex=2/1) to give 4-((1S,4S)-4-ethylcyclohexyloxy)pyridine (cis isomer) and 4-((1R,4R)-4-ethyl cyclohexyloxy)pyridine (trans isomer).

¹H NMR (More polar isomer, 400 MHz, CDCl₃) δ 0.90 (3H, t, J=6.8 Hz), 1.26-1.38 (6H, m), 1.59 (3H, m), 1.99 (2H, m), 4.60 (1H, m), 6.80 (2H, dd, J=4.8, 1.6 Hz), 8.40 (2H, dd, J=4.8, 1.6 Hz). LC-MS Calcd. 205.15, Found 205.96 [M+H]⁺.

¹H NMR (Less polar isomer, 400 MHz, CDCl₃) δ 0.91 (3H, t, J=7.2 Hz), 1.04 (2H, m), 1.24 (3H, m), 1.43 (2H, m). 1.88 (2H, m), 2.14 (2H, m), 4.24 (1H, m), 6.78 (2H, dd, J=4.8, 1.6 Hz), 8.39 (2H, dd, J=5.2, 1.6 Hz). LC-MS Calcd. 205.15, Found 205.98 [M+H]⁺.

### Example 43

### Preparation of 4-(4-ethylcyclohexyloxy)-1-tosylpiperidine (cis, trans mixture)

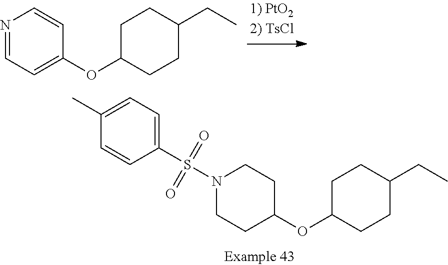

To a stirred solution of 4-(4-ethylcyclohexyloxy)pyridine (150 mg, 0.731 mmol) in ethanol (3 mL) was added platinum oxide (50 mg, ⅓ weight equiv.). Concentrated sulfuric acid (0.04 mL, 0.731 mmol) was added to the mixture. The mixture was hydrogenated overnight at 40° C. (balloon). The mixture was cooled to 0° C. and was neutralized with 6 N MOH (0.49 mL, 2.924 mmol). The inorganics were filtered off and the mixture was concentrated under reduced pressure. The residue was dissolved in DCM (3 mL) and was treated with Et₃N (0.2 mL, 1.462 mmol). 4-Toluene sulfonyl chloride (280 mg, 1.462 mmol) in DCM (1 mL) was added dropwise at 0° C. The mixture was stirred for 20 minutes at room temperature. The solvent was evaporated and the residue was purified by Prep-TLC (Hex:EtOAc=5:1) to give the desired product as a colorless oil. ¹H NMR (400 MHz, CDCl₃) δ 0.83 (6H, d. J=7.6 Hz), 1.141-1.26 (10H, m), 1.31-1.42 (7H, m), 1.59-1.70 (7H, m), 1.79-1.86 (4H, m), 2.43 (6H, s), 2.84 (4H, m), 3.28 (4H, m), 3.37 (2H, m), 3.49 (2H, m), 7.31 (4H, d, J=8.4 Hz), 7.65 (4H, d, J=8.4 Hz). LC-MS Calcd. 365.20, Found 366.13 [M+H]⁺.

### Example 44

### Preparation of 4-(4-ethylcyclohexyloxy)-1-(methylsulfonyl)piperidine (cis, trans mixture)

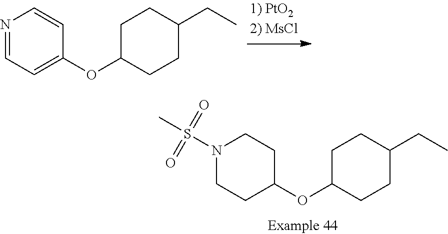

According to the similar procedure in Example 43, the desired product was obtained. ¹H NMR (400 MHz, CDCl₃) δ 0.87 (6H, d, J=7.2 Hz), 1.21-1.49 (12H, m), 1.69-1.77 (8H, m), 1.81-1.88 (4H, m), 2.78 (6H, s), 3.17-3.26 (4H, m), 3.33-3.38 (4H, m), 3.57 (4H, m). LC-MS Calcd. 289.17, Found 290.01 [M+H]⁺.

### Example 45

### Preparation of 3-(4-(benzyloxy)cyclohexyloxy)-6-(4-(methylsulfonyl)piperazin-1-yl)pyridazine (cis, trans mixture)

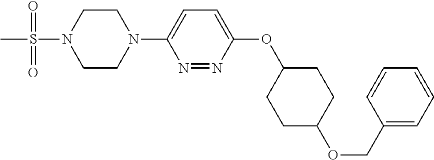

### Step 1. Preparation of 1-(methylsulfonyl)piperazine

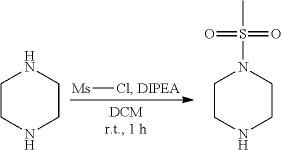

To a stirred solution of piperazine (4.51 g, 52.4 mmol) and DIPEA (3.05 mL, 17.5 mmol) in DCM (40.0 mL) was added methanesulfonyl chloride (1.36 mL, 17.5 mmol) at 0° C. After being stirred for 1 hour at room temperature, the reaction mixture was extracted with DCM and water, dried over anhydrous MgSO₄, filtered and concentrated in vacuo to give the desired product (2.40 g, 84%) as a white solid which was used for the next step without further purification. ¹H-NMR (400 MHz, CDCl₃) δ 2.79 (3H, s), 2.98 (4H, m), 3.21 (4H, m). LC-MS Calcd. 164.06, Found 164.95 [M+H]⁺.

### Step 2. Preparation of 4-(benzyloxy)cyclohexanol

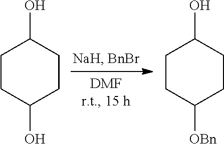

According to the similar procedure of step 1 in Example 37, the desired product was obtained. ¹H-NMR (400 MHz, CDCl₃) δ 1.31 (2H, m), 1.63 (3H, m), 1.96 (3H, m), 3.38, 3.49 (1H, m), 3.70 (1H, m), 4.52 (2H, m), 7.27-7.34 (5H, m).

### Step 3. Preparation of 3-(4-(benzyloxy)cyclohexyloxy)-6-bromopyridazine

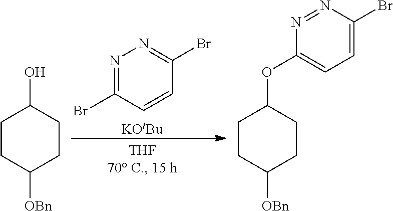

To a stirred suspension of 4-(benzyloxy)cyclohexanol (50.0 mg, 0.242 mmol) and KO′Bu (40.8 mg, 0.364 mmol) in THF (3.00 mL) was added 3,6-dibromopyridazine (115 mg, 0.485 mmol) at room temperature. The reaction mixture was stirred at 70° C. for 15 hours. The residue was diluted with EtOAc and washed with water and brine, dried over MgSO₄, filtered and concentrated in vacuo. The residue was purified by column chromatography on SiO₂ (Hex:EtOAc=3:1) to give the desired product as a white solid. ¹H-NMR (400 MHz, CDCl₃) δ 1.58 (3H, m), 1.78 (2H, m), 2.04 (2H, m), 2.23 (1H, m), 3.46, 3.57 (1H, m), 4.55 (2H, s), 5.27, 5.36 (1H, m), 6.81 (1H, m), 7.26-7.37 (5H, m), 7.45 (1H, m). LC-MS Calcd. 362.06, Found 362.80 [M+H]⁺.

### Step 4. Preparation of 3-(4-(benzyloxy)cyclohexyloxy)-6-(4-(methylsulfonyl)piperazin-1-yl)pyridazine (cis, trans mixture)

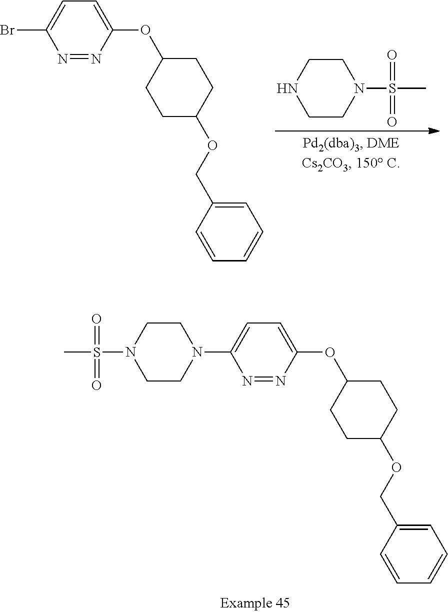

A mixture of 3-(4-(benzyloxy)cyclohexyloxy)-6-bromopyridazine (200 mg, 0.551 mmol); 1-(methylsulfonyl)piperazine (90 mg, 0.551 mmol), Pd₂(dba)₃ (50 mg, 0.055 mmol), X-phos (52 mg, 0.11 mmol) and Cs₂CO₃ (270 mg, 0.827 mmol) in DME/H₂O (5.0 mL/0.2 mL) was heated at 150° C. in a microwave for 3 hours. The mixture was diluted with EtOAc (50 mL) and MgSO₄ (1.0 g) was added. The inorganics were filtered off and the filtrate was concentrated. The residue was purified by column chromatography on SiO₂ and further purified by preparative TLC to give the desired product as a white solid. ¹H NMR (400 MHz, CDCl₃) δ 1.25-2.29 (16H, m), 2.81 (6H, s), 3.35 (8H, m), 3.47-3.56 (2H, m), 3.66 (8H, m), 4.56 (4H, m), 5.17-5.24 (2H, m), 6.85 (2H, m), 7.02-7.05 (2H, m), 7.27-7.37 (10H, m). LC-MS Calcd. 446.20, Found 447.20 [M+H]⁺.

### Example 46

### Preparation of 1-(cyclohexylmethyl)-4-(4-(methylsulfonyl)benzyl)piperazine

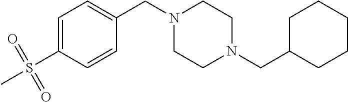

### Step 1. Preparation of 1-(chloromethyl)-4-(methylsulfonyl)benzene

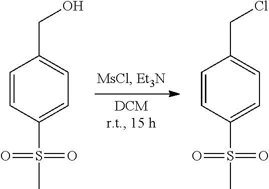

To a stirred solution of (4-(methylsulfonyl)phenyl)methanol (1.00 g, 5.37 mmol) and Et₃N (1.50 mL, 10.7 mmol) in DCM (20.0 mL) was added methanesulfonyl chloride (0.464 mL, 5.96 mmol) dropwise at room temperature. The reaction mixture was stirred at room temperature for 15 hours. The solvent was removed in vacuo and the residue was diluted with DCM (100 mL). The solution was washed with water and brine, dried over MgSO₄, filtered and concentrated in vacuo. The residue was purified by column chromatography on SiO₂ (Hex:EtOAc=2:1) to give the desired product (691 mg, 62.9%) as a white solid. ¹H-NMR (400 MHz, CDCl₃) δ 3.06 (3H, s), 4.64 (2H, s), 7.60 (2H, d, J=8.0 Hz), 7.95 (2H, d, J=8.40 Hz).

### Step 2. Preparation of 1-(4-(methylsulfonyl)benzyl)piperazine

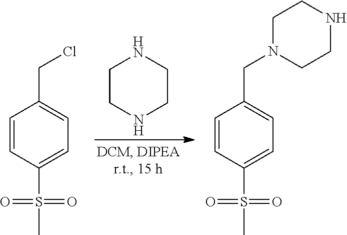

To a stirred solution of piperazine (1.45 g, 16.9 mmol) and DIPEA (2.95 mL, 16.9 mmol) in DCM (20.0 mL) was added 1-(chloromethyl)-4-(methylsulfonyl)benzene (691 mg, 3.38 mmol) at room temperature. The reaction mixture was stirred at room temperature for 15 hours. The mixture was concentrated in in vacuo and the residue was dissolved in DCM (500 mL) and washed with water and brine, dried over MgSO₄, filtered and concentrated in vacuo. The residue was recrystallized in DCM to give the desired product (701 mg, 82%) as a white solid. ¹H-NMR (400 MHz, CDCl₃) δ 2.42 (4H, m), 2.89 (4H, m), 3.05 (3H, s), 3.57 (2H, s), 7.55 (2H, d, J=8.4 Hz), 7.88 (2H, d, J=8.4 Hz). LC-MS Calcd. 254.11, Found 255.05 [M+H]⁺.

### Step 3. 1-(cyclohexylmethyl)-4-(4-(methylsulfonyl)benzyl)piperazine

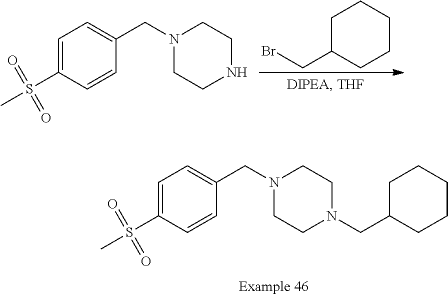

The mixture of 1-(4-(methylsulfonyl)benzyl)piperazine (200 mg, 0.786 mmol), (bromomethyl)cyclohexane (0.56 mL, 3.150 mmol) and DIPEA (0.41 mL, 2.359 mmol) in THF mL was heated at 130° C. in a microwave for 1 hour. The mixture was concentrated in vacuo and the residue was purified by column chromatography on SiO₂ (EtOAc as an eluent) to give the desired product as a white solid. ¹H-NMR (400 MHz, CDCl₃) δ 0.87 (2H, m), 1.13-1.26 (3H, m), 1.46 (1H, m), 1.64-1.77 (5H, m), 2.12 (2H, d, J=7.6 Hz), 2.47 (8H, bm), 3.06 (3H, s), 3.58 (2H, s), 7.54 (2H, d, J=8.4 Hz), 7.88 (2H, d, J=8.4 Hz). LC-MS Calcd. 350.20, Found 351.21 [M+H]⁺.

### Example 47

### Preparation of (4-(5-ethylpyrimidin-2-yloxy)cyclohexyl)(4-(4-(methylsulfonyl)benzyl)piperazin-1-yl)methanone

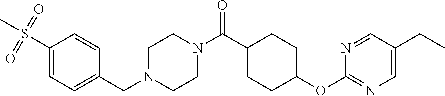

### Step 1. Preparation of 4-(5-ethylpyrimidin-2-yloxy)cyclohexanecarboxylic acid

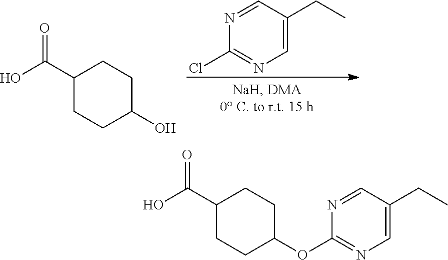

To a stirred solution of 4-hydroxycyclohexanecarboxylic acid (500 mg, 3.47 mmol) in dry DMA (6.00 mL) was added sodium hydride (207 mg, 55% dispersion in oil, 4.73 mmol) at 0° C. After being stirred for 30 minutes at room temperature, a solution of 2-chloro-5-ethylpyrimidine (450 mg, 3.16 mmol) in dry DMA (4.00 mL) was added dropwise to the mixture at 0° C. After being stirred for 15 hours at room temperature, the reaction mixture was quenched with saturated aq. NH₄Cl and extracted with EtOAc. The separated organic layer was washed with brine, dried over anhydrous Na₂SO₄, filtered and concentrated in vacuo to give the desired product (750 mg, crude) as a reddish oil, which was used for next reaction without further purification. LC-MS Calcd. 250.13, MS Found 250.97[M+H]⁺.

### Step 2. Preparation of (4-(5-ethylpyrimidin-2-yloxy)cyclohexyl)(4-(4-(methylsulfonyl)benzyl)piperazin-1-yl)methanone (cis, trans mixture)

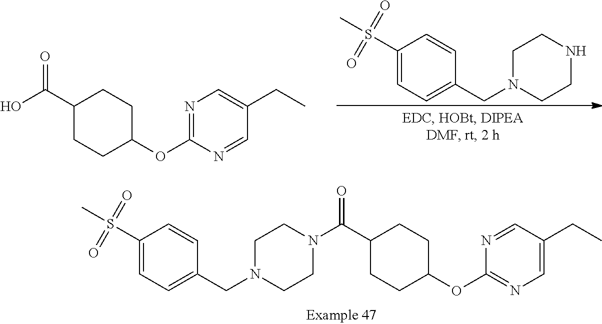

To a solution of 4-(5-ethylpyrimidin-2-yloxy)cyclohexanecarboxylic acid (100 mg, 0.393 mmol) in DMF (3.00 mL) was added EDC (190 mg, 1.07 mmol), HOBt (133 mg, 0.977 mmol), DIPEA (0.240 mL, 1.38 mmol) and 1-(4-(methylsulfonyl)benzyl)piperazine (200 mg, 0.799 mmol) at room temperature. After being stirred for 2 hours at room temperature, the reaction mixture was extracted with EtOAc/H₂O. The combined organic layers were washed with brine, dried over Na₂SO₄, filtered and concentrated in vacuo. The residue was purified by column chromatography on SiO₂ (CH₂Cl₂:MeOH=10:1) to give the desired product as a white solid. ¹H-NMR (400 MHz, CDCl₃) δ 1.24 (6H, t, J=7.2 Hz), 1.57-1.86 (8H, m), 2.04-2.32 (8H, m), 2.43-2.53 (10H, m), 2.56 (4H, t, J=7.2 Hz), 3.07 (6H, s), 3.49-3.58 (4H, m), 3.60 (4H, s), 3.61-3.65 (4H, m), 4.94-5.19 (2H, m), 7.56 (4H, d, J=8.4 Hz), 7.91 (4H, d, J=8.4 Hz), 8.32 (4H, s). LC-MS Calcd. 486.23, Found: 486.98 [M+H]⁺.

### Example 48

### Preparation of 4-(5-ethylpyrimidin-2-yloxy)-N-(4-(methylsulfonyl)benzyl)cyclohexane carboxamide

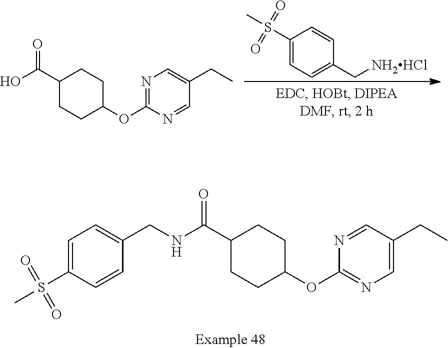

To a stirred mixture of 4-(5-ethylpyrimidin-2-yloxy)cyclohexanecarboxylic acid (125 mg, 0.499 mmol) and (4-(methylsulfonyl)phenyl)methanamine hydrochloride (110 mg, 0.499 mmol) in DMF (3.00 mL) was added EDC (190 mg, 1.07 mmol), HOBt (133 mg, 0.977 mmol) and DIPEA (0.240 mL, 1.38 mmol) at room temperature. After being stirred for 2 hours at room temperature, the reaction mixture was extracted with EtOAc/H₂O. The combined organic layers were dried over Na₂SO₄ and concentrated in vacuo. The residue was purified by recrystallization from Hexanes and EtOH to give the desired product (130 mg, 69%, cis and trans mixture) as a white solid. ¹H-NMR (400 MHz, CDCl₃) δ 1.17 (12H, t, J=7.6 Hz), 1.59-1.67 (8H, m), 1.75-1.85 (4H, m), 1.95-1.99 (4H, m), 2.30-2.36 (2H, m), 2.55 (4H, t, J=7.6 Hz), 3.19 (6H, s), 4.36 (4H, d, J=6.0 Hz), 5.14 (2H, brs), 7.49 (4H, d, J=8.4 Hz), 7.87 (4H, d, J=8.4 Hz), 8.43 (2H, brs), 8.45 (4H, s). LC-MS Calcd. 417.17, Found: 418.12[M+H]⁺.

### Example 49

### Preparation of 4-(5-ethyl pyrimidin-2-yloxy)-N-(4-(4-(methylsulfonyl)piperazin-1-yl)-4-oxo butyl)cyclohexanecarboxamide

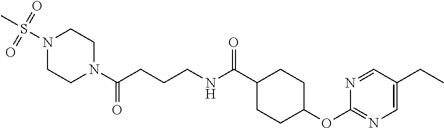

### Step 1. Preparation of 4-chloro-1-(4-(methylsulfonyl)piperazin-1-yl)butan-1-one

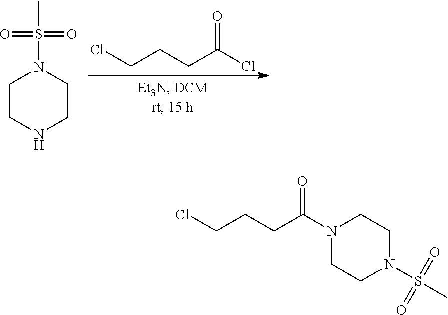

To a stirred solution of 4-chlorobutanoyl chloride (0.041 mL, 0.365 mmol) in DCM (5.00 mL) was added 1-(methylsulfonyl)piperazine (40 mg, 0.244 mmol) and Et₃N (0.041 mL, 0.292 mmol) at 0° C. The reaction mixture was stirred at room temperature for 15 hours. The reaction mixture was diluted with EtOAc and washed with brine, dried over MgSO₄, filtered and concentrated in vacuo. The residue was purified by column chromatography, on SiO₂ (Hex:EtOAc=1:1) to give the desired product (47.3 mg, 72.3%) as a white solid. ¹H-NMR (400 MHz, CDCl₃) δ 2.12 (2H, m), 2.54 (2H, t), 2.81 (3H, s), 3.23 (4H, m), 3.64 (4H, m), 3.75 (2H, m). LC-MS Calcd. 268.06, Found 268.97 [M+H]⁺.

### Step 2. Preparation of 4-azido-1-(4-(methylsulfonyl)piperazin-1-yl)butan-1-one

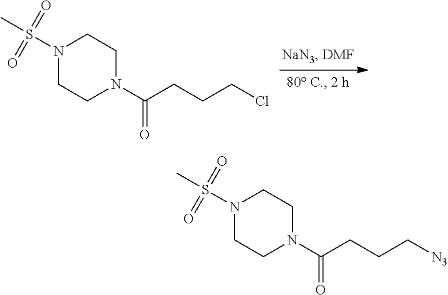

To a stirred solution of 4-chloro-1-(4-(methylsulfonyl)piperazin-1-yl)butan-1-one (100 mg, 0.372 mmol) in dry DMF (2.50 mL) was added sodium azide (35.0 mg, 0.538 mmol) at room temperature. After being heated for 2 hours at 80° C., the reaction mixture was diluted with water, extracted with EtOAc. The separated organic layer was washed with brine, dried over anhydrous Na₂SO₄, filtered and concentrated in vacuo to give the desired product (87.0 mg, 85%) as a white solid. ¹H-NMR (400 MHz, CDCl₃) δ 1.95 (2H, p, J=6.6 Hz), 2.44 (2H, t, J=7.0 Hz), 2.80 (3H, s), 3.21-3.27 (4H, m), 3.41 (2H, t, J=6.4 Hz), 3.60 (2H, t, J=5.0 Hz), 3.76 (2H, t, J=5.0 Hz).

### Step 3. Preparation of 4-amino-1-(4-(methylsulfonyl)piperazin-1-yl)butan-1-one hydrochloride

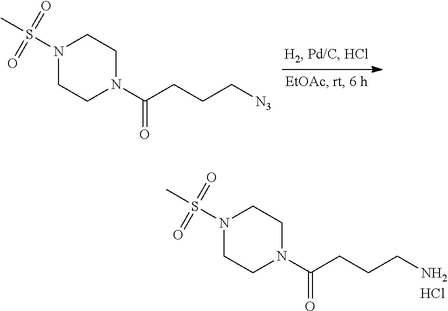

A mixture of 4-azido-1-(4-(methylsulfonyl)piperazin-1-yl)butan-1-one (87.0 mg, 0.316 mmol), Pd/C (17.0 mg, 20 wt %, 0.0158 mmol) and hydrochloric acid (0.100 mL, 0.316 mmol) in EtOAc (2.00 mL) was stirred at room temperature for 6 hours under hydrogen atmosphere (balloon). The mixture was filtered through Celite and the filtrate was concentrated in vacuo. The residue was purified by recrystallization from Hexanes and EtOH to give the desired compound 14 (37.0 mg, 41%) as a white solid. ¹H-NMR (400 MHz, DMSO-d₆) δ 1.77 (2H, m), 2.46 (2H, t, J=7.2 Hz), 2.80 (2H, t, J=7.6 Hz), 2.90 (3H, s), 3.08 (2H, t, J=5.0 Hz), 3.13 (2H, t, J=5.0 Hz), 3.52-3.57 (4H, m), 7.76 (3H, brs).

### Step 4. Preparation of 4-(5-ethyl pyrimidin-2-yloxy)-N-(4-(4-(methylsulfonyl)piperazin-1-yl)-4-oxobutyl)cyclohexanecarboxamide

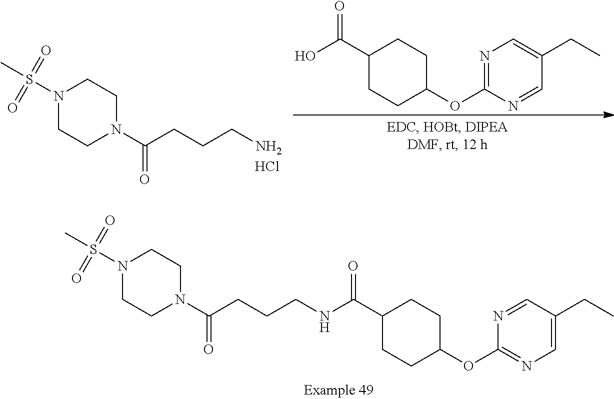

To a stirred solution of 4-(5-ethylpyrimidin-2-yloxy)cyclohexanecarboxylic acid (37.0 mg, 0.148 mmol) in DMF (1.50 mL) was added EDC (60.0 mg, 0.338 mmol), HOBt (45.0 mg, 0.331 mmol), DIPEA (0.100 mL, 0.574 mmol) and 4-amino-1-(4-(methylsulfonyl)piperazin-1-yl)butan-1-one hydrochloride (37.0 mg, 0.129 mmol) at room temperature. After being stirred for 12 hours at room temperature, the reaction mixture was diluted with EtOAc and washed with brine, dried over Na₂SO₄ and concentrated in vacuo. The residue was purified by column chromatography on SiO₂ (DCM:MeOH=10:1) to give the desired product (40.0 mg, 64%, cis and trans mixture) as a yellow oil. ¹H-NMR (400 MHz, CDCl₃) 1.17 (6H, t, J=7.6 Hz), 1.63-1.69 (4H, m), 1.77-1.81 (2H, m), 1.85-1.96 (8H, m), 2.11-2.16 (4H, m), 2.20-2.27 (2H, m), 2.39-2.43 (4H, m), 2.57 (4H, t, J=7.6 Hz), 2.82 (6H, s), 3.23-3.30 (8H, m), 3.31-3.34 (4H, m), 3.57-3.60 (4H, m), 3.66-3.80 (4H, m). LC-MS Calcd. 481.24, Found 482.16[M+H]⁺.

Biological Evaluation Methods

The synthesized analogues may be evaluated by methods known in the art. [eg. Arena Pharmaceuticals inc. (e.g. WO 06/083491A2, WO 07/120702A2, WO 08/005576A1), Metabolex, Inc. (e.g. WO 08/038238A2, WO 09/123,992A1, WO 10/008739A2), Prosidion Limited (e.g. WO 08/081208A1, WO 09/050523, WO 10/001166), and Smithkline Beecham Corporation (e.g. WO 08/070692A2), which are incorporated herein by reference].

A non-limiting evaluation content includes,

1. Stimulation of cAMP

2. Insulin Secretion (Islet Perifusion) assay

3. Oral glucose tolerance test

4. Incretin (GLP-1) measurement

5. Triglyceride level test

6. Gastric emptying

7. Body weight loss

8. Whole body insulin sensitivity test

Camp Assays of Selected Examples

| Examples | <200 nM | >200 nM |
|---|---|---|
| 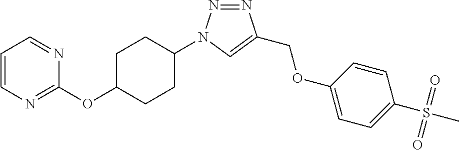 | | √ |
| 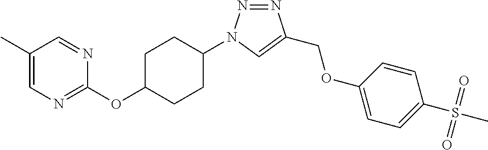 | | √ |
| 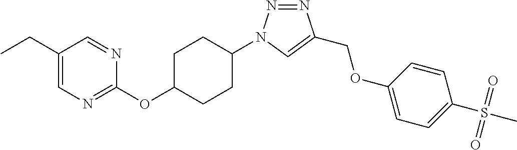 | | √ |
| 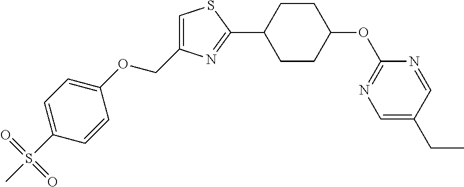 | √ | |
| 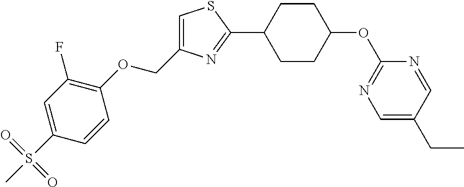 | √ | |
| 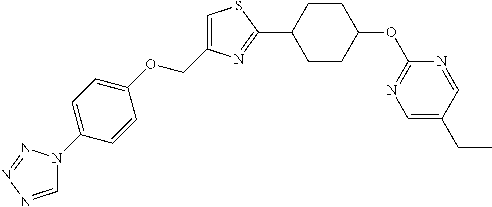 | √ | |
| 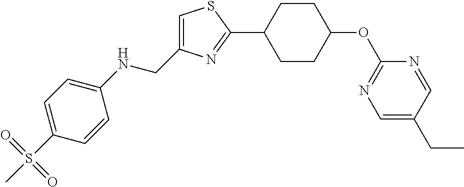 | | √ |
| 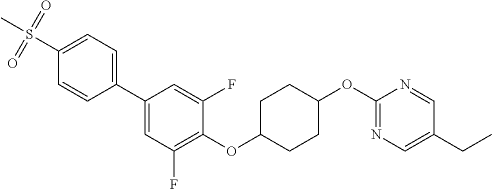 | √ | |

1. A compound of formula I, or a pharmaceutically acceptable salt, isomer or prodrug thereof: 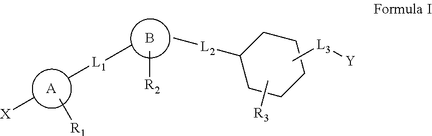
wherein,
A is 5˜6 membered aryl, heteroaryl, cyclic alkyl and cyclic heteroalkyl;
B is (C)ᵢ where i=0 or 1 independently, and C is independently chosen from 3˜6 membered aryl, heteroaryl, cyclic alkyl, and cyclic heteroalkyl;
L₁, L₂, L₃ are linkers having the formula of (Z)ₖ where k=0 or 1 independently and Z is independently chosen from —(CRₐR_{b})ₘ—, —(CRₐR_{b})ₘO(CRₐR_{b})ₙ—, —(CRₐR_{b})ₘCO(CRₐR_{b})ₙ—, —(CRₐR_{b})ₘS(CRₐR_{b})ₙ—, —(CRₐR_{b})ₘSO(CRₐR_{b})ₙ—, —(CRₐR_{b})ₘSO₂(CRₐR_{b})ₙ—, —(CRₐR_{b})ₘNRₐ(CRₐR_{b})ₙ—, —(CRₐR_{b})ₘCONRₐ(CRₐR_{b})ₙ—, —(CRₐR_{b})ₘNRₐCO(CRₐR_{b})ₙ—, —(CRₐR_{b})ₘOOC(CRₐR_{b})ₙ—, —(CRₐR_{b})ₘCOO(CRₐR_{b})ₙ, —(CRₐR_{b})ₘNRₐCONR_{b}(CRₐR_{b})ₙ—, —(CRₐR_{b})ₘNRₐCOO(CRₐR_{b})ₙ—, —(CRₐR_{b})ₘOOCNRₐ(CRₐR_{b})ₙ—,
where, m, n=0˜5 independently;
X is hydrogen, —CN, —F, —Cl, —Br, —NO₂, —CF₃, —ORₐ, —CONRₐR_{b}, —NRₐCONRₐR_{b}, —NRₐCOR_{b}, —CORₐ, —SRₐ, —S(O)Rₐ, —S(O)₂Rₐ, —CH₂S(O)₂Rₐ, —SO₂NRₐR_{b}, CH₂SO₂NRₐR_{b}, C₁-C₅ alkyl, C₁-C₅ alkenyl, C₁-C₅ alkynyl, optionally substituted aryl, heteroaryl, cyclic alkyl or cyclic heteroalkyl wherein the substituents are independently represented by X;
Y is chosen from hydrogen, C₁-C₁₀ alkyl, C₁-C₁₀ alkenyl, C₁-C₁₀ alkynyl, aryl, heteroaryl, cyclic alkyl, cyclic heteroalkyl, heteroalkyl, haloalkyl, perhaloalkyl, —COR_{c}, —CONR_{c}, —SO₂Rₐ and —SO₂NRₐR_{b}, any of which may be optionally substituted with R_{d};
R₁, R₂, R₃ are substituents independently attached to A, B or cyclohexane ring which could be chosen from hydrogen, —CN, —F, —Cl, —Br, —NO₂, —OR_{c}, —NRₐR_{b}, —COR_{d}, —CONRₐR_{b}, C₁-C₅ alkyl and each A, B or cyclohexane ring can have above substituents from one up to four of them independently; and
Rₐ and R_{b} is independently hydrogen, C₁-C₆ alkyl, or OR_{c}; R_{c} is hydrogen, C₁-C₁₀ alkyl, C₁-C₁₀ to heteroalkyl; R_{d} has the formula of (W)ⱼ where j=0˜2 independently and W is independently chosen from hydrogen, —CN, —F, —Cl, —Br, —NO₂, —OR_{c}, —NRₐR_{b}, CORₐ, CONRₐR_{b}, NRₐCONRₐR_{b}, C₁-C₈ alkyl, C₃-C₈ cycloalkyl, C₂-C₈ alkenyl, C₂-C₈ alkynyl.

2. A pharmaceutical composition comprising the compound, pharmaceutically acceptable salt, an isomer or prodrug of claim 1 and a pharmaceutically acceptable carrier or diluent.

3. The pharmaceutical composition according to claim 2, which further comprises another antidiabetic agent.

4. A method for treatment or delaying the progression or onset of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, or hypertension, the method comprising administering to a mammal a therapeutically effective amount of the compound, pharmaceutically acceptable salt, isomer or prodrug of claim 1.

5. A method for treatment of type 1 or type 2 diabetes mellitus, the method comprising administering to a mammal a therapeutically effective amount of the compound, pharmaceutically acceptable salt, isomer or prodrug thereof of claim 1 alone, or in combination with at least one agent selected from the group consisting of another antidiabetic agent, an agent for treating diabetic complications, an anti-obesity agent, an antihypertensive agent, an antiplatelet agent, an anti-atherosclerotic agent and a hypolipidemic agent.

6. A compound selected from the group consisting of 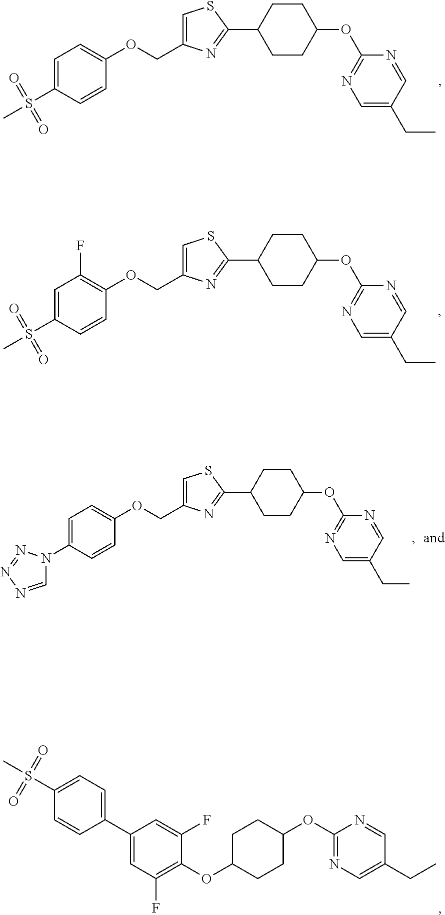
or a pharmaceutically acceptable salt, isomer or prodrug thereof.

7. A pharmaceutical composition comprising the compound, pharmaceutically acceptable salt, an isomer or prodrug of claim 6 and a pharmaceutically acceptable carrier or diluent.

8. The pharmaceutical composition according to claim 7, which further comprises another antidiabetic agent.

9. A method for treatment or delaying the progression or onset of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, or hypertension, the method comprising administering to a mammal a therapeutically effective amount of the compound, pharmaceutically acceptable salt, isomer or prodrug of claim 6.

10. A method for treatment of type 1 or type 2 diabetes mellitus, the method comprising administering to a mammal a therapeutically effective amount of the compound, pharmaceutically acceptable salt, isomer or prodrug thereof of claim 6 alone, or in combination with at least one agent selected from the group consisting of another antidiabetic agent, an agent for treating diabetic complications, an anti-obesity agent, an antihypertensive agent, an antiplatelet agent, an anti-atherosclerotic agent and a hypolipidemic agent.
